SEQUENCE LISTING (2 sequences)

SEQ ID NO: 1 - DNA, 36853 bp, Homo sapiens
taaacagatagaaacgctgtgctccaggcagatgccataacaaacactggctctagatctaccatgggttatatgggtccttagttaagattagatatggagtcacagtgtgccagaatttcagggagagaggaaagtggaagggctttccactaagccttttgaactactaggtacccctcgttttacatctggttatctcatttaaatctatgacgttttaaaatacttattaccatttaagacatgagaaaaattaagtttagaaacggctagatagctcacgctagaaaggaaggactccaaattttaaccaagggcagatgtgcatggaggccaagtcatttccttcccatgctctcatactgacccaacaaacccattctgcacgtgagaaaactgaggcccggaggagggaattcacacacgtttgaagccaagctggggccagaaagtagatcggctgatctccaaacctccacgtcaaaggtcttcccaggaggacatatgccttcaaggattttacaatgtataccaccctacaagtgatgggagagaggggaaaaaagcaaccgactaaggagaaaagcaaggagcagaaaaaggagcaaaagaggagctgtcagaacggtgtgaggagagtgggaaaggagccgcctccttttgcctctcttcctacttattaacaaaacagaacgactatccaacgtatttcagggagctaagtcttagccagcttcagctgtgttttaacccttagctaaatatagacaaggctaaggcaggccaggtgtacacttcaggaagacgtggaaattcccagggcggaccaaaacttgagctgttccggcggtagtgatttgaggtcagtttgaaagagaagttgagaaagcggccgaggggcgaatttggctgcactacacgctcgccacaaagaggaagggccgccggccgccggggccgagtgcgcttccagcccaggcggagcggtagacgcaacagcctccgcccgccacgtgacccgtagtgttgtggtctggcccgcgtgggtggcgggagcgccgagagggcgggggcggcgatggggcgggtcacgtgatggggtttaaatctcccgcagccggagccgcgggggcgccagtgccgcgcgtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgaggtgcgcagggtgcgggcggccggaactgcgcatctcctcctccctctccccgggctcctactggcctgaggttgagggcggctgggggctcggggcaggctccgcggcgttcccctccccaccccggccctccgttcagccgcgctcctccggggctgcggttcctactgcgcgagctgccagtggattcgctcttttcctccgtccgtggcccgcctgggcggccttgttctttccgcagcagccaggtcgggagactctcgcagtcgctttaaaataagtttctctccccctcttactcttttagcatattgcttttgttagagctttttttttctttttctttattttttattttcattgcttttctcctctaccccccagcacacttaagtctgcaactttttggctgtcccgttggtctgcgttttcaattgctttttgcaaacttgacacctgtgcagtttgctttttttggtctatatttttatgactgaaattacaatttttgaaagaggaaagctgtttctatagtcacagcttgcaaaatttaaagaacgtggctgttcctgaagacattaccattctgtaatgcagttaggagtgggtatgggagagagagtacttttagagctgttcatattacttttattctaatcttgctcttgtgcggtaatggtgtttgggctaaacatgctttttttgtatgtatgtagtattgagacacaaacttaacacttctagcataatgaagcagatatgttgcagttgtcatgttcttgaaggtttctttttttttttttttttttttttgagacggagtctcgctctgtcgctaggctggagtgcagtggcgcgatctcggctcagtgcaacctccgcctcccgcgttcaaccgattctccttcagcctgccgagtagctgggactacagacacccgccaccacgcccagctaatttttgtgtttttagcagagacggggtttcaccatgttggccaggatggtcttgatttcttgacttcgtgatccgtccgcctcggccgcccaaagtgctgggattacaggcgtgagccaccgcgcccggtcaggtttcttatttttgatctgaggagtttctatccttgataggtgttcatctttatcaatatttgggtcaggccgggcgcggtggctctttcgtgtaatcctagcactttgggaggccaaagcgggaggatctcttgagcctaggagtttcaggccagcctgggcaacatagcgagacctcctctctactaaaagtaaaaaaagcgggacgtggtgtcgcacacctttagtcccaggtactagagttcgaggttgcagtgagctgtgatcctgccactgcactccagggtgggcaacagagtgagaccccgtctcaaaaaaaaaaaaaaaaaaaagtaatatgtgtttttagggactgatttagtaaagagaccctgatttcaagcaacacagttgagtactaatttattatttacctaataataattgtgttattaatactgtttatttaatagaggaaacaatgcagaggtacttgtagtattgtgtaagtttaacgtgcgcattgccaaggttatatttgaatgacgtttttatgtaaatgtccatttattacacgtttcacgaacgctattgtttgtgattgtatgatacattctagctagattattatcttttctttttttttttccttttctttttttttaaaaggtggagtttcgctcttgttacccaagctggagtgcaatggcgtgatctcggctcaccgcaacctccgcttcccgggttcaagccattctcgtgcctcagcctcctgagtagttgggattacaggcatgcgccaccatgcccggctaatttttgtattattagtagagacgagatttctccatgttggtcaggctggtctcgaattctcaacctcaggtgatccgcctgcctcggcctcccaaagctctggaattagaggcgtgagccaccgctcccggctatcttttctgttttttaagtagacttaatttatttttagattttggctgaaactgagtgagcagaattcctaaagatgaagaaactgttccaatgaacagtgcaagctgaccccataggcatgctttacacacgccgtgtaaatgttaaatgctgtactcgatgaaaatgattgctctataaccgttcatacattttaggtgcatgttgttttgatagccttgactgacttggtttcttttgcagataaccttctgttcggtgatgaaattatcactaatggttttcattcctgtgaaagtgatgaggaggatagagcctcacatgcaagctctagtgactggactccaaggccacggataggtatggctcagagctgttaattttagagagtaaatgtacggtttttggtttgtttgttttgagacagagtttcgctcttgttgcggaggctggagtgcaatggcgcgatctcggctcaccgtaccctccgccttccaggttcaagcgattctcctgcctcagcctccccagcagctgggattacatgcatatgccactacgcccggctaattttgaatttttagtagagacggggtttctccatgttggtcaggctggtcttgaactcccgacctcaggtgatccgtccgcctgggcctcccaaagtgctgggattacaggcatgtgccaccttgccccacctcacttgtcccctttttgtgtagtgttcttcatccggttagaatatcttcctccctcacttcatattccttgttttttcactttattgtacacttaccacttcttgaaaagctctgtatcttactttttaaattgtgttgtttcctggtctagaatataagttctacgggagcaggaatttttgactgacttgtatactcctgtcttttcagtgcctaaaaaagtgcctggcacatagtttgtgctcaataaatatttaataaatatgtgaatgaaatgggaaagtatttcgatgtagaaagcattatcagttttttataccaaaggtataaccatcttgaaagtggatctttttgtgttctcactcttacaaatggcttgattatttaaacatagtgaagatcagtttctctttggtgtctgtaatttatttatttatttatttttgttttctcaagcctagggagctggtttgagcaatctcagggtaaaagaaaatgattagttctcagaacatcaacattgtatgtattatcttggaagatgggttaagatcaaattgaaaaattaccggaaaaaaattaagatggcagttatgtatagattttcatttctttttcctgtgtggttttaatgataagtagtacaggcttttaggactggtacgatttttctatgtcgtagaacccttttgtaaatacttttggtattatggtgtgtgtttctagatattaaccataagaacttaattctggttgttaattaattagactcttgaaatatatacttacagctgaagggggtcttcccaactcttcattagatcttcctaatctcattgggatatagcattaattctgagtgtaccgagaatgaaataccattaaattgcattttcttactttgcaaaaaaccctcacagaatgctaactcattacttcagaaaataaatgagattttgaattacttgataagttgactttaagccttttcccccttattgtaggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtatgtaagactagtacatggggaggtcgtatatgtattttcttatgccttttccaagtaggaaacatttttctggtttagaaggatttatccttacatgataatggatgtttgggaactgccaaaaactgagtgcagcagcagttgctaaattagttcagatttttaaaacctctccagccttaaaaaaatatttgcttctgcttatttatagcacagtgggtaaatggagttaaggcaagatttctacctacattatatcatagctcctaaaggagatgcagggaattgaaactctgcttttgttaagggatgtcagtctgatggagaaattgggtatttgttagatctttatgagaaactggaaacctaattggtattcacaagtgaaaagcttagtgttgggaagagtaacagattcctaccttccctccatattaattaagatgatgtgattgtggcgacttgacattttctatttgatctaccttagggttgcagctaattagttacctaagactattgcatagtgtgtgttttatacagagtatattacatgttagcaaccagagcaactgctgtagtaaaattttgtcctgttttctgccatttcgagttgcgaaagggtgttacataatcttagatgatcatcaggagtatgttttgctcacagataacgatgtagacaagtttgtttaacttaaattgaccttgactccaggtcaaattggagtcaactctggtaagatgatgaaacaataacttctgaaatctctatttgaaatgtcaggatttttttgtacgattagtgggcacgtctcaaagaaatgaagaattgaaatattatacaccttttttttttttcttttttttggatacagggtcttgctgtgttgccctggctggagtgcagtggtgtcatcatggctcactgcagtctcagcctctcctggcctgagtcttgctctgttgcctaggctggagtgcagtggcaggatcttggctcactgcaacctctgcctcctgggttcaagcaattctcttgcctcagcctcctgagtagctgggattacaggcgcacaccaccacacctggctaatttttgtatttttagtagagatggggtttcaccatattggccaggctggtctgaaactcctgaccttgtgatccgcctgccttggcctcccaaagtgccaggattacaggcgtgatccaccgtgcctggctatacattgttttttttaatgaaaatagagaattgttgtagtattgaggtaatagaatcctaactaaaggagcaatttctttggaattacttctctgtaatagaaattcttggctggtggagtggctcatgcatgtaattccagcactttgggaggctaaagtgggaggatcacttgagccctggagtttgagatcaccctgagcaacatagaccctgactctacaaaaaattagcctggtgtgctggtgcgtacttgtagtcccagctgctaaggaggttgaggtgggaggattgcttgagccggaaaggttaaggccataatgagccatgatggcaccactgcactccagcctgggcgtcagagcgaaactgtctaaaaagaaaaaaaacaaaaagccgggcgcggtggctcacacctgtaatcccagcactttgggaggctgaggcctgcggatcacgaggttaggagatcgagaccatcttggctaacatggtgacaccccgtctctactaaaaatacaaaaaatcagccggtcgcggtggcaggcacctgcattcccagctaatcagtaggctaaggcaggagaatggtgtgaacccaggaggcggagcttacagtgagccgagatcgcaccactgcactccagactgggcgacagtgtgagactctgtctcaaaaaaaaaaaaaacaaaaaaaaaaagaaaaaattcttccctacttaaaataatttgttgaaatggtttattttcttctcaagttgcatttgctttaatttgttggctaaatgcttaaaaggcttaaaattggcctgacttaaaaattccttatctgtgttaaagatggaatatgaggtatatttaattttactcagttatttgggagttttatttctgaaataagggtagggttgtatttttatttcttaaaagaagtttcagactaaaattttctttctcaactcttacctaattatatggtaagagagctagctagttcctataaaggtagaagatttaattttacaaattatgccatgcacattttaggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggtactatgaactcttctggttgtttctttggccttctctcatgaaaaagtattttgttcacatacagccaccttaaggttatcgttcattgtttagtaaagtgaatgctgctactgtggcggagtaagatcactcattatggctagaattcctttattcctagaggaggactattatctacttcattttaggagtgagcttattttcaaagagatagttcatatttttaaaatttgcactgcagcgatggtcgttattctgcctgggcttttttaagaggtttgcacaccatataaaagtaacataacttgtgatttttaatattttattagagattgtaaaggttataacatcactttggtgtttcgtagtcaagttttaacataaggatgtgcctgaaaaatcatttgtaattagagaatgggaagattcttgggttgcatttttgtcagcaaattgcagaggatcattattctgctctagagttgcaccgtccagttcagaagccactagccacatgtggctgttgagtacttgaaatgtattgatatgtgcaataagtgtagaagacatattggattttagagatccagtatggaaaatacaaagtatttcattagttttattcatcaaatattaaacaaatattggtttttatatggaaaaaatacttaaaattaattttgaattcttttgaaatacttttgatattgggttaaataaaatgtattttgggctgtgcgtgttgcctcatgcctgtaatttcagcactttgggaggccaaggtgggaggatcagttgagcccaggagtttgagaccagcctgagcaacataatgagactccatctctacaaaataaataataaaatttgctgagcatggtggtgtgcacctgtagacccatccactcagtagtctgaggtgggaagatcccttgagctcaggagctcaagactgagtgaaccatgatctgccattgcactccaccctgggcaacagagcaagagtgtttccaaaatatatgtatgttttttgaagtttactttttttttttttctttttttgagaagtagtcttcctttgtcccccaggctggagtgcaatggcatgatcttggctcatagcaacctctgcctcccaggttcaagtgattctcctgcctcagcatcctgagtagctgggattacaggcgcccgctaccatgcccagctaatttctgtatttttagtagagacagggtttcaccaggttggccaggctggtgttgaactcctgacttcaggtgatccaccctcctcggcctcccaaggctctgggattataggcgtgagccaccgcgcctggccagttttcaccttaatgtggttactaaacaatttaaagttatatgtatgtctcacattatgtacctattgtacagctctgctttagcatagaaagtttaataatgcattacaccattctcaagagattgagtcattagaaaactgttttctttcctgtatttcagtcttttggaaagaaattacaaaatttgttattgttaaacttggaggtatatatatttgaaagagtcattttattttattttactttaagtgccgggatacatgtgcagaatgcgcagatttgttaacataggtatacatgtgccatggtggtttgctgcatctatcaacctgtcatctaggttgtaagccccacattcattaggtatttgtcctaatgcttttccaccccttgccccccacctcctgacaggcctctgtgtgtggtgttccccaccctgtgtccatgtgttcttattgttcaactctcacttatgagtgaggacatgtggtgtttggttttctgttcttgtgttagtttgctgaggatgatggcttccagcgaaggagtcttgtattagtggctttttccccacctaatcgttagaagttgtgaatagggacttctttaatgaatccagaagttaatgaacctagcttttttttttttttttttggagacggagtctggctctgtttcccaggctggagtgtagtggtgtgatctctgctcaccatacaacctccacttcccgagttcaagagattctcctgcctcagccttccgagttgttgggactacaggcgcgtgctgccatgcctggctaatttttgtatttttggtagagacggggattcactatgttggccaggttggtcttgaactcctgaccttgtggtccgcccatctcagcctcccaaagtgctgggattacaggtgcgaggcaccgcgccccgcgccctgccgatgaacctaaattttaactaaacagtggccttatctactttcagaccatgtggtatatttacatgactacaggagaagctttgctaattcagaataaattatgttacttaaattggcgactgtctttaaaaaaaaagtgattttttggatgcagtagctcgtgcctataatcccagtgctttgggaagctgagataggaagatctcttgaactccggagtttgagaccagcctgcgcaacacagtgagaccctaatcactacacccctccaccccatgtaacttttgttttatccaggttgtggtatattaaatgggcattagtgtaaagtgggaaaattatattaattcttgaatatgattactaaactgaatttgaaaagttttcaggctatcaagagaatttttaacttaaaacttatataatttgaactttttacttcatatatccgtaatgatgatggtcatctatatctagcttttagagcagacaactggttcatacctggattaaataaataatgtaaagttattttttgttaattatggattagtgaagatttactgttttaacctactcctgctagtggcactactgcatggttttgaagaccagtgaagtatgacttaaaggtttcttgaattggagctaggacactggcatttataaaatctccacgtagcgcagacattgacactatcagaagaccagcaagtaactagaagttactttgatcttaaatcaactacaaaaaacttgactcactagttatggagaatacattttttattgttaaactgaaaaaaaattcagtcacttataaggtgtagcctcttctaatcctgtttatataaatttatttattattattattttttgagatggagtcttgctctgttgccaggctgtagtgcattggcgcgatctcggctcactgcatcctccacctcccgggttcaagcaattctcttgcctcagcctcccaagtagctgggactacaggcacgtgccatcacgatcagcacttttagtagagacggggtttcaccatgttggcaaggacgtgtctcgtcgtgacctcgtgatccgcccgtttcagcctcccaaagtgttgggattacaggtgtgagccacaatgtccagctgataaatttaattttgcttttctgagttttcatttatggtaatggttaaatcagctagcctttacagttgttactcacatataattctttgtccatcctttaattctcacattgggaactgactaaaaaaagaaagcttccagtctgggcacagtggctcacacctgtaatcccagcactttgggaggccgaggtgggtggatcacctgaggtcaggagttcaagaccagcctggccaacacagcaataccccgtctctaataaaaaatacaaaaatcagccaggcttgggtggcgggcgcctgtcgtcccagctacgcaggaggctgagacggggagaattgcttgaagccaggaggtggaggttgcagtgagccgaaagcgcttaaggagaaataagtaaggacaaagagtgcaggatagtatcctgacacggaggatggggagggtatgtaaatattgtcgatattttactgaatttctaaatattacaagaatagaatattcataggataacctaagcttttctgtgagtcagtgtactaggcagaaagttcactaaagtggtttatgatttttgctacttatgagaaaacattaaaaatgaagccatgaaaaggaaatattggtgaaatttagatacttcagaaggaaagagagactttcttttttttttggaaagaatttctataccatctattatatcattgattgtatgtgattattgagaattaacttgttttttttttttttgttttttttttttgagacagttttgctcttgtcacccaggctggagtgccgtggtgagatctcggctcactacaaccttcacctcccgagttcaagcaattctgctgcctcagtctactgagtagctgggattacaggtgcccatcaccacgcccagctaatttttctaattttagtagagacgggatttcaccatgttggtcaggctggtctcgaactcctgacctcaggtgatccgcccgcctaggccaaagtgctgggattacaggcgtgagtctcccacgcccagcccttaaatttcatttagactggaaatttcaagtaatggagaggtgtgttaaggaagttattgctctgttagagcagtttcttaaatttggcactattgacattttgggctggatttttctttttgtttctgagatagtttcactctgtcgagccatcacagctcgttgcagccttgaactcctgggctccttgaacaggaggctcctgcctgagcctcctgagtagctgagaccataggcacgtaccatatacctagctaaatgtgtttttgactttcttttttctttgtagagtcaagttctcactatgttgtccaggctggtcttgaaatcctgggttccagcaattctcctgcttagccttacagagtgtcgggattgtaggcataggccactgcacccggccttgggctgatatttatttgctatggggaactttcctgtgcgttgtaggatgtttggcaatgtccctggccggccaggtgcagcggctcacacctgtaatcccagcactttgggaggctgaggtgggcggatcacttgaggctgggagttcagaagagaccagccaacatggtgaaaccctgtctctactaaaaatacaaaaattagccgggtgtggtgacacacatgagaatcacttgaactcaggaggctgagtttgccgtgagctgagatcgcgctgctgtactccagcctgggtgacaaagcaagaatctgtctcaaaaaaaaaaaaaaaaaaaaaaaaaattcgctggtctccacctacgtttgtatccccaccccaagtcctgacaataaaaaatgtcttcaaaatgttccttgcaaaattgctcattgaggctaactgaggtggctcacaactttaatcccagccctttggaaggctgaggctggcagatcacttaagcccaggtgactggccctggccaacttggcaaaaccctgtctctactaagaatacaaaaaatagccagacgtggtggtgcacacctgtaattccagctactcgggaggctgaggcagcagaatcacttgaacctgggaggcggaggttgcagtgagccgagatcgcaccactctactccagcctgattgacagagtgagactccatctcaaaaaaaaaagaaattactctctgagcaccagtgtgttacagtgtgcttagctgtggtaccactttggattctttcacacaagaacagttaaggccagttttaaagctgtggggccatttctgaactgtattttgtttttttttttttggagatgaagtttcactcttgttgctcaggctggagtgcgatggtgtgatctcggctcacttgcaacctccgcctcccaggttcaagcaatctcctgccttagcctcttgagtagacttgattatgggatcttggctcactgcaacctctgcttcccaggttcaaacagttctgccttagcctgctgagtagctgggattacaggtgcccgccaccatgcttggctaattttgtatttttagtagagacggggtttcaccatattggccaggctggtctcgaactcttgaccttgtgattcacccacctctgcctcccaaagtgctggtattacaggcatgagccactgtgcctagccaattttttgaatttttagtagagatggggtttcatcatgttggccacgctggtctcgaactcctgacctcaggtgatccacccaccttggcctcctaaagttctgggattacaggcgtgagccacttcgcccagcctgcactgtgttcttatcctgcataatgacttaaattgataataagttgtggtcatggttctcagaaacctaaaataattcactgatgttaataaaaatgagtggttgtatttagagtcaaggcgaaactgaattaggatgtataaaataactttttttttttttttttttttttaagtaggactgttgttcggctgggcgtggtggctcacgcctgtaatcccagcactttgggaggccacagcgggcggtcaaaagtttgagaccagcctgaccaacatggtgaaaccctgtctctactaaaaatacaaaaattagatggatgtggtggcgcatgctgtaatcccagctactcaggaggctgaagcaggagaatcacttgaacccaggaggcggaggttgcagtgagctgagatggcaccactgcactccagcctgggcgagagagtgagactctgtctcaaaaaaaaaaaaaaagaaaagaaaaaaaaagaaaagggactatactatgttcgaagagtgtagtttctgtcccatactgaattcactgttctttccctagtgattaaaatacaagaaaaggtagagggagaaacacgaactgaattttatgaacttaattccaagaatgtatttttacatttgttcaaaaaattattactttattgataatatgcaaacataccagattatgtattaatagttaaaaatttgagttctagaacaagcttattaaaaatcaacgtcagtaaaaagaaaatagatggtagtcacagtgccattcatctgaaaataatgttttaatactcaacatgtatatggattatatatctatacgtttatagtattgtagctggatttatgttacatttttagcataaaatttggcttttgctgggcccagtggcttgcgactgtagtcccagcaactcaggagggtaaggtgggaggattgcttgaaccaaagagtttgaggctgcagtgagctgtgacctcacctctgcacaccagcctgagtgacagagcaagacccatccctgaaaagaaaaaacttggctgttttactttgctttcaatttgtagtatattctacttaatataataaacttactttctgtgaggtgcttgaatactttttaatggttatttatgaagtggtgaactgtaattctgtatttgtacatttggatgtacaaatagtgttgctacagttgtccttttttggtatgtctccagtgttcttaggataaattcctgaaagtgaatttcagttttgaatttcagagaaacatgtcaaagttcttctttaagcctttaagtctcctgatatgttttgtcgtattgcagcttggaaaggttgaaataaaatcttctattagggctgggtgcagtggctcatgcctgtaatccgagcactttgggatttagctgatttggctgatttatcatgccactgccacctgtatccacccaaaatccacaaaaaccccaggatacattcttagcattaaaaaaaaagttatataaatatatataattatatatatatttgagacagggcctgtctctgatacccagacgtgtgcagtatctttcaaaaatcatatatctatatatagtttgccccttccttcagtctttgctgcccctaatatctgtccttaatggttaccatggtgaaactcttggaaatcattcagggggaaaaaaaatttgtgttgggataggggagggatgtcagcataagtttttaatacttgataaatgattgccaacagaatcaaaaacaccttgttttctatcttttttcaccatataatattcttgagaataatttcgtaacagcacttaacagatataccttatttttcaaagaaggctgtgtagcattctattttaaagttgtttcacagtatatgagtctcctattgataggtatctgcatttttgtctttttgatctagggttgtttttaaggcagtttaaaaactatagtaagtaaaagaggccaggtgcggtggctcacacctgtaaacccagcactttgggaggccgaggcgggtggatcacttgaggtaggagtttgagaccagcctggcaaacatggtgaaacccttctctactaaaaatacaaaaaattagccggccgtggtggcaagcgcctgtagtcccagctgttcggaaggctgagggaggagaatcgcttgaactcgggaggcggagattgcagtgagccaataatatgctgctgtactccagcctgggcaacagagcgagactctatctcaaaaataaatacaattttaaataaaattataaaaataaagcagcacccagctttattttttatttatttatttattttttgagacagagtcgcattctgtcgctcaagttggagtgcagtggtgccatcttggctcactgcaacctctgcctcccgggttcaagtgattctcccgcttcagcctcccaggtagctgagattacaggtgtgtgccaccacgcccagctaatttgtatttttagtagagatgggggtttcaccatgtttgccaggctggtcttgaactcctaacctcaggtgatccgcccaccttggcctcccaaagtgctgggtttataggtgtgagccgccgcgcttggctcaacatcttattgtttgagacagggtctccttctgttatccaagctggagtgtagtagtgtgatcttacagctcactgcagcctccaactcttgggctcaaatgattctcctgtctcagcctcccaagtagctagtactacaggtgtgcaccaccatgccctgctgtttctttgtttttttttatatggacagtcttgctatgttgcccaggctggtctcaaactcctgagctcgagtgattctgctgcctcagctactcaaagtgctgggattgtaggcgttaactatcaggttaggcctgttctgtttttattagcgtggatgactgctggttactactggccgtttgtatatccttgaattacctctttgtttttagccatttttctccatttgggtgctttttcacagtgacttctgggaatattatgttcttttcttttgatttacctttcaaattttatttttatggatatataatagttgtacgtgtacattatatattcttgaagagttaggcttattgggattccagcctttagtagtcccatgaaggtgagcaaactgaaatgtacttactagtcttagtcatttggttcgtaatatgtaatagaaatggtgcttatcctaaaagaaaaatagaaaacttgagggtgaaggatcagaaattgaagacagtaaatactgcatagaggtttaaatgctagggtttttaggttttcttttttttttgagatagtcttgctctgacacccaggctggactgcaacctctgttccctggcctcaagcgatcctcccaccttagcctcctgactagctgtgaccacagatgtgcaccaccatgcccggctatttttttttgtatttttggtagagatggggtctcgccatgttgcccagtccagtgctggattttcatatcaaaacagggctctgctacttacttgctcttgtgaccaaaatggcaagttaggtttgttttgtatttgttttcatctgcatcggggtatatgttcacagctggatcagcaaactttctgtagtaaggtagggcctggtagtaaatatgtagtatgctttgcagactgcatagggtctctgtcatatgttctttgttttctttacagccttgaacaaattaaaaaacccagtcctcagccttgtctgtagcataataaagaggaaattaaaagtgcttatgttatacgtggtacatactaaaatttattaagtggcgactggcagtttaactaattgacattctcttgtgttaggttaatacaggggttggcgtactatggcctcagtccaaatgtggcccactccatgtttttgtataggctgtgagctaagcattaagcattaatttattacttaaatcaacaaataaagtttatgtggtgtacaacatgatgttttgatgtgtgtacattatggaatggctaactcaagctaatattcattacttcacatacttttttttggtggtgagaacatttaaaatgtactcttaataattttcaggtatacaatatttgtttttgtttttgtttttgttttgagacgaagtctcgctcttgtccccaggctggagtgcagtggcgctatcttggctcactgcaacctccgcctcccaggtacaagcgattctcctgcctcagcctcccaagtagctgggattacaggcgcctgtctccacgcccggctagttattgtatttttagtaaagacagggtttcaccatgttggccaggctggtctcaaactcctgacctcaggtgatccgcctgcctcagcctcccaaagtgctgggattacaggcgtgagccaccgcgcccggctacaatatgttcttaattatagtcagcaatagatctcctgaacttattcttcccagcgtcttcccaatcccacctccaccccctgctaataattaattttaaatgactggggaaaaaattgaaagaactgtttcatgacacttgaacattatataaaattttcatgtcagtgttcataaatacaaacttttattagaacatagccatactatttatatattgaaatacagttgaatttgtcatatgaccaatttattagtattgattaagatgttgagcagtttattcaagtgtggttttgtggaatgaaagagacaggagggattatgaggactctccttagcaggattgatgcatttcactttttttgacaggaaggaaagtttaggttctattttctcagcaactctacttaactgcctccatctgaactatatataggagggggtggatatcagcaaagattaacatcagtaaattttgatgaattctctaaaaattctgatttttaaagagggaattttggctgggcacagtggctcacacctgtaatcctaagcactttgggaggccaaggcaggtggatcacctgaggtctggagtttgagaccagcctggccaacatggtgaaaccctgtctctactaaaaacaaattagctgaatgtggtggcgcgcacctgtagtcccagctccttgggaggctgaggtgggagaattgcctaaacccaggagaggtggaggttgcagtgagccgagattgtgccattgcactccatcctgggtgacagagcgagactctcaaaaaaaaaggtgggtgtgaattttgttatgttgtgcgggctggagtgtagtgcccatccattggcacgatcatagagtactacagctccctaactcccagactcaagtgattctccctcctctgtctccccagtagcaggaactacaggcctgtgccaccatgcccagctttcttagtcttgagggactgcatttacacctatgccttttactctagggtcttttattcatattgctagaaatttggagaagaaagaaaggcataatctctggagaaaagccattatttctgcagatagttctttttttttttgagatggagtctttctttgtcacccaggctggagtgcagtggtgtgatctaggctcactacaagctccacctcctgggttcacgccattctcctgccttagcctcccgagtagtgggactacaggcacccgccaccatgcccagctaatttttttttgtattttttagtagagacggggtttcaccatcttagccaggatggtctcgatcttctgaccttgtgatccgccgacctcggccacccaaagtgctgggattacaggcatgagccaccacatccggccaatttctgcggatagttctaagctttagttttgaccacgttggttgcctatggccaattcctggattatttgcatgtttccagacacagctatgtgaaagcagggtttgttttaatagtgttttgagcctttgattccagttctgcttccactcttggtctataaggatccctatggcagcaacagaatggcccagccctcatgattggttgcttaaaaaggctttaggatattcaaactgggtggcctgtcttgtttggaataggactaagcatgtcacactttcaccatggaagagttaatccatattctatctgtaagttgtatctccagtcattggttttcaacaagtggtcctatcttaggaggagtggtatattagaacagtccttccagagtaatttttttcattatttcagattttttagttttttttgtttgttttttttttttttttttttaaaggctctcactctgtcacccaggctggagagcagtgatacgatcatagctcactggaacctctacctccctgggctcaggtgatcctcccacctcagccttctgagtttctgggactacaggcatgaaccgtgaggcccagctaatttttgtgatttttgtgttttttgtagagtcagggtttctccatgttgcccaggctggtctcgtactcctggtctcaagtaacctgcccgcctcggccttccaaagtgctgggattagaggtgtgagccaccacacctgtactactacttttagtttgaacactatatttaaatctaataattcagacatggcccatttcatacttgacagaaagtatgagtcaaagactcaaatcattcagattctcacatatgtaaaacgtacgcttggccgggtgcagtggctcacgcctgtaatcccagcactttgtgagaccaaggtgggcggatcacctgaggtcaggagttcaagaccagcctgaccaacatggagaaaccccgtctctactaaaaatacaaaattagctggggtggtggcacatgcctgtaatcccagctattcaggaagctgaggcaggagaatcacttgaacccgggaggtggaggttgtggtgagctgagatcgcgccattgcactccagcctgggcaaaaagagcgaaactctgtctcaaaaaaaaaaaaaaaagcaggcttataaaataaaacaaaaatgtcccttatgatttgtcttaaaggtagttgaattaataagagctaaatctagtgtcccatacatctttgtgtttcttactctaccctttagaaagggtgggggtggacgtggataaggcacacagttaattcagggacggtggagtcttggtacttgctctttttccctgaccttgctttggagttattttattttttaaattctgcatttggcatgctattatcacttgcttactactataggtcctattttatctgttgcctagcatattctcatagtgcctaaaatactctaaaagctatttccagagggagctttaatgagacatacaatgcttaatctctcaaatgtaaatgtactagaacaaagcattctggggaagaaaacagtggttgatttaactgaactcacttttccaaaacagtggcttttgattttctgtaaacattgactttattaagcttttcccttgctactatatgcactctctatttcttaaattgcaaagagttattcatgaaatttgtgataaatatttgaatctaaggcaacgtttttggattctttatacgaggtttttagcagcatgtgtacatgtatgtgatagctgttaacggaattcacttaagttttacttagaagcctgggtagtagaacctagaagttgcagtgggaatttttttttaatttgaaagattaaataggtaaaaagtcatctttaatctctattaaacttttttgtataatgtcagaagtgaaagttccccacatctgcttacagcctgtttgtatccttcctgacttgattgattaaatggacatctctatatacatatgcatataatttcaatttttgcttagtaacaaaaacaaaagttacacatactggtgactaatttgcttttttcacgtatgtcatgaaccattttccagggcagttcatctagatctccctcaattctttttaatggctacatattccactattatggatgtaccatatttatacaaccggttccctattgatggacatatggttgtgtctagttttttgctagcattgtgatagattagagcctgtaatgaatggtaaggaagtaagtagatgatagcactactactttgaaggaaaaagtagagaataaacttcctttgccatagtcacttactaaatggaatttaataaaaacactgtcaaaagttgggaggaccaaaattgatactttttctctgatctttttgccatgtgtatatctgaattctttgtttttaaagaagaaacagcattgaagcattatttggggggaaaaacacacacacaaaatccagcaactcagcattcatgagcaactctatactataccagtatgtgcctgtgcagtggaaggaaaacaattttggtaaggattaaaactttagctttaaacttccagcaggttgatattctaatgaatgataaatcaaaaaaaaattttaaatattcttgtattgacagtgcttttttttttaaatcaccctaccttgatatctgtaattttagtttaaactttcccatttttctttaaagaaaaaaatctgaatttgcagccaacaaaaattagatatcctaaggttgtattacttctgacttttattaaaatatcaacatttcttagagatgtttatgggccgactttgtctttttcttcaaggggccaagttcactaattgctgagttttatgcatatgacagcaaccgtccttttgtaggtgtgtgtcgcatccatctagatactttaaaatgctcatctatttcatttttaaaattatgtgtgtgggattatcagtatttttttgttaaacatatgacatctgtagtttatttcactaatgtaaattttttctaccatttgcttgatacaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttagtaaacttcagagtggttttctgtaatttattttagttttataggaagatatttcctataaagctgactgccatcgagaagtggagataaagcattatttaatcatgttatctcatttatcgataacctcagaaaagtagaaaacaaaaataataaaaacagaagtattggccttgacagttaattatagaaaacctcagatattaatttgcttttgatttcaaaagatgctgcagttgcatgcattcaattattttaaataatcttttctggctgagtgcagtggctcacgcctgtaatcctagcactttgggaagccgaggcgggcagatcccatgaagccaagagttcgagaacagcttggccaacatggtgaaatcccaactctactagaaatactaaaattagccgggcatggtggcaggcgcctgtaatcccagctactcaggaggctgaggcacgagaatcacttgaacctgggagttggaggttgcagcgagccaagattgcaccattgcgctccagcctgggagacagtgagactctgtcaaaaaaaaaaaaaaaaaagaaaaagaaattctgaatacaagagtagtattagctgttaatgaagaaatgtgacatctttagtttatgaaactaaaagaactggatagttgagatgtacaggattcagagattcagaaatgtttaaaacaagtatcaacaggccattaggtatctaaagtgtttctaagaactgccgagctaaggtaatagagttggaactgtccaactctgcaggattttagccctggaggagtgagctgttacagttttgttgaaaagagtagctttagaaggcatcattatattagtgtctcagagattgagaatcatattcattctatgtataaatatataaattcttcatcccctagattctcctgtagtatatcacaaaatctgcagtgtgttctgaggtttaaaatcaaagttaaaaacaaaaacaaaaatccttaaaccctcttaacatttgtgatgttaaactttataacgtttgtggtgtgttcaagaaacagaaatacttctttaataaagcatatatatgttgtttgtttttaggttcctttgcaacagcatcttgcctgatttgtaaatacaaagttgactgtgaagctgtacgaggagatatttttaatcaggtaatttgttgcccatattttaggaattgttcatgtctctgaagtatttcttcttttgcctcaaaatccttttttacccctttaaagtatatatggtacagaaagattcaggaagaaaatagttagcatttgggaattttggtaaaatacacgagaacctttcaataccttatatagaaaacagccctataaaggctcttcaaattatgaaattggcttcttagtatctaagccgaacataaaatgtcagatcataagcaagttgttgttgtagtagtttttctccttcctctccctttttgtgcctgtcagatttggaccttaaattagttatggtaatctaaaattatttcatacatgttcattgccaaaaaatataggaaaatttggcagaaaataaaaattatccagaaatatgtcctgtgtgcttagcaatatatggaggtaatcatgtcattacgaaagaggaagaatagtatattaaaatggagtcattggtcgggtgcagtggctcatgcttgtaatcccagcactttgggaggacgaggtgggtcgatcatttgaggtcagaagtgagaccagcctggccaacatggcaaaaccccgtctctcctaaaaatacaaaaattagcctggcatggtggcatatgcctttaatctcagctgcttgggaggctgaggcaggagaattatttgaactcgggaggtggaggttgtagtgagctgagatcatgccacttcactccaggctgggcaacagaacaagactccatctcaaataaataaaatggaatcattttatttgacaaatgtctatttttttttgaaaggatcaattcccatttagtgtatatacacacattatgtttttcctcttaatatatgatggactcttcaatgtcaagtgtttttctacatagatattataacaagagcatagtatttcattgtttcatagtatgttaataaactacccatcattgacagttgctgtttatccatagcttttctgttttgttttttttttaataattctgaaatgtattcttagaggtatggaaatgttgactatttctaacttgggcttactctttgcttctctacctcaaccaaaatctgaaaatatgtaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagtaagttggtgatggtttttggagaacatttctatatataatgtcatgggttgtgggtctgtataatagacgctagtaatcttaactctgcttctgtttgaaagagtggtgaagagctaattttagaaattgtttgtttgtttgtttattgagatggagtttccctcttgttgcccaggctggagtgcagtcgcatatcttggctcactgcaacctccgcctcccgggttcaagcgattcttctgcctcagccttctgagtagctgggatcacaggcgtccaccaacatgcctggctagtttttgtatttttagtatagaccgggtttcaccatgttggccaggctggtctcgaactcctcagctcaggtgatccgcctgtctctgccttctaaagtgctgggattataggcatgagcccccatgcctggccagaaattctttatttttagtagagatgaggtcttgttgtgttgcccaggctggattcgaactcctgggctcaagtgatcctcctgcctcagccccttgagtagctaggattacaggcacgagcctccacatctggctgaactgtttttttaggtggcattgttcattgagactggtgaatctgacattttgatggggggtggagggttgtcaaaacgcaagtaatgttggtggctcgtgcctgtaatctcagcactttgggaggccgaggtaggtggatcgcttgagggcagcagtttgagaccagcctggccaacatggtgaaaccctgtctctactaaaaatacaaaaattagctgggcatagtggtgtgcacatgtagtcccagctacttgggaggttggggcatgagaatcgcttgaactcaggaggcagaggttgcaatgagccgagaatgtgccactgcactccagcctgggtgacagagtgggactctgtctcaaaaaatcaaaaacaaacccagaagtaatgctaaactctacttctaatttatgtgaaaaattaagacaaaggtagagttcaacttagactttttggtggaatgtgtttttaatgttgcaagggttagcatcaattaatttatgaaatggtcctttatccttatattttttgattacagttttgatctttcaaatttaattgatttcacatttaataaattcaaatgtatagtttggtagaagtgtaacataccatgtaagatggaatttgggggctcagaatgattggttcattcttgtagtaacagccatgccataacggtgatatgtcgatatagtttacccaaaagggtgtgtatataaaagtgttcacataaaatttaaatccctattactctcaggaaaatttctttgtcatatatattctcatactgtctgtttcctcctctagtttagaatcagaggaggagagagagagatttcagttgcatcagatgtgtcctttcataagtgagggaaagaggagaggaaaaatattaaatccctagccagagaattgaattagaccccacatcagcagtcaaggcagccagagtaaacagttggaagaaacatggagtcaagctcttcaattttgtgtctatcccatatctcgtggctttagcagttaactcagtcttacctaattagctgtgaaattctgttaaagtagaatacaagacaatttgcaattaaatgaaacatgaaaatttaatatacaaatctctttgttcttgagttgctcttcttaaagcaaaatcaaaggcactctgcttaaaagcttttctttttattttttagagacgggtgtcttgctgtagctcaggctagagtgcagtggtgcagtttcagctcactgcagccttcaactcctggggacaagcagtccttccacttcagcttcctgagtagttaggaccagaggcgcacaccatgcttggcaaatttttaaatttttttttgtagaaacgggattcactttgttgcccaggctggtcacaaactcctggcctcaagtgatccttcctcctctgcctctcaaagcattaggattacaggcatgagctaccatgcctggtccttaaaaacttttctttcaaaagctttctgtgggatagtatgatgtgttgcaaagataattaaaaaaagaaacaggtttataatagcgtagaatatgtttatatgactttttccctccccccctcccctccctccctcccttccgtttgtccttccttccattcatccttccttctgttagtccttccttctgtccatccttcccgtccgttcttccttccttccttcgtccatccttctgtctgtccttccttccgttcatctgtccgtccttccttcctcccaccctccctccctccctcctatcctccctccctccctcattgcccaggctggagtgcaatggcatgatctcggctcactgcagtctcccgtgttcaagcagttctcctcccaagtagttgggattagaggcatgcgctaccacacgcccggctaattttgtgtttttagtagagatggggtttcaccatgttggtcgggttggtcttgaactcctgacctcaggtgatccgcctgcctcggcctcccaaagtgctgggattacaggcgtgagccactgtacccagctcaccttctttatatcaggagctacttaagtagaacatttatgtaccaagaactcttctgtgcacttgactaactcatttaatcctcaccacagtcttgtggagaagtactattatcatccccattctgcagacaaggaaatttgaggttcagagtgggaaagtctgggaagattgctcaggggtaaccaggtgataggagcagaacttgagcgttttataaaagacacttaactgcccatctgcttgcttgatgaaatgtaatggcttggttaagtatttagtgcatgggtcttttttgggaatttggagctcaagcccttgttggatttttgcataatgtatctgttgtggttttattagcttacttcctcctccctttttctaactcttatttttcaccctattttaggttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaagaaaggcagtcggaccattttgaaagtataaatgtcataacagtatttccaaaaaattagctatttcggcaggttaatcgatagggtagctttatgtagttgattctgtttagagaaactgtacagttcgtaatcagaaaggtaaatcttctggtatcttaacatgatatggagaaggaagtgtttaatagtgctctgtatgttgtgtttctctaggggatggaaaaataagaatgggttattagctggcagaaatgatgacagatttgagtgcttactgtaggtcagctgctttacatatgttacctgattttaatctgaggtaggaactattactcttcttcaggtgggaaaactcaggaccatagaggttaaatacctcatgcacagtaactactaagaagtggaagaaccagattaaaatccagtctatttatgcttagagcctgcccccttaactactatgcggtgttgtctcagatgtagaacacatctttgttttctctgaggacttagaaaaaacacgcgtgattcttctccactgaatataggaatagtttctaccctgtttagaacatgcatagtctttatcaataagtgttaactgacatgtgaaatcactttctactctttggaagccgatgttttaagtccaagtagtctgtatggtggaaattcctaacttatgttaaaaaaaagaaaaccactgtaacttagctataacagtcttatataacttagaactttaagtctcattccatttaccatacttggcgaaaactgacaaattttagacaactgtatttgagtattccagtcatttgctgtaaatacattttagttcagttgaataaacctttggcctttttcatattaggcactgtggtaagttatggagacacagtgattaatgttgtgtataattataaatgtgagtgatactcattaatactttggtataggtgctgctgaagaacaaagtactttattattgttttgaacaaaacatcacacaaaaggtagattttttttttttttttttttttttttttttgtgacggagtctcactctgtcgcccaggctggagtgcagtggcacgatctcagctcactgcaacctctgcctcccaggttcaagagattctcctgcctcagccttctgagtagctgaaattacaggcatgcgccaccttgctcggctgattcttgtatttttagtagagacggggtttcgccatgttggtcaggctggtctcaaactcctgaccccatgatccatccacctcggcctcccaaagtgctggaattacaggcgtgagccacggcgcccagccccaaaaggtagattctacttggagattagattacagaaggctttctaaggagcaaaatatttaacaataagtagggatttaaaaagagcagacgtgttcacaggggaaactagaaaaagcatagagggatgcttacgtttgcaaatcgtggcagaagtcagaaagtagaaaaattgctactgacttagatacacagttgtctctagcgtatacaaagtctacctatgcactaagacttgcactgggaacttttcttgaatgtcattttgacagatgtgtcaaacggacaatctctttgcccaggaaacagagttaacctggaacagtcatttttttttaaatttatttattttttgagacggagttttgctcttgttgtccaggctgaagtacaatggtgtgatctcggttcacggcaacctccgcctcccgggttcaagcgattctcctgcctcactctccagagtagctgtagctgagaccacaggcgcatgccctcatgctcagctattttttttttttttttttttagtttttgtagaaacaaggtcttgccacattgcccaggctgataccaaactcctgggctcaagcagtctgccagccttggcctcccaaagtgctgggattacaggtgtaagccactgtgccctgcctgtggtgtcttgggaaactcatgagtactatgtgtctgttgtaatagagggaaataagtggttttcacagtgatttgtagtggactgtgaaattttagggattcaggtcagagttgtcacacaggttgtagtcagggtgagaactgggtcatgatgcagtatgaaaaagttcgagagccactttggagagaacttgagataggccacctaccagtgtggtaaccaggcttttgagaattcgtctgggatacggtacaataaatactacatctattatgtgtgaagagatgttaagttagggacatactgtgaattcaaggatagaaaacttttccatcagtttttagggatcctactctttcacttaaaccccaaatggccaagctaggattgatttggtgtgctgtagaaagaacttcattggtattcatggattcacattacatcttagaggagttttcaaaagcgtcttagactgtatgtgtatatacacacacattctgaagcagtaggtgggtcttggggcctgagatctcgggtgaatctaaatttaggttcacaggtgatactgtagattcacagtgtctacagagtacactatgaatttgtggtgactacattattgacaaaatattttaggtttataatcagaaaaaagttaaaatagttaatgaagatgctttaaaagcctgtgtactttagagaagctacttaacacaaattgggtatctaatgtaggctgggctggatacttcattttcatcaaatctttttaaaataattggtgaaataacctttattgaatatggttttctacatttttcacacttccctccttcatagggttgtgaaaatttatttcatattctagatgaggaaattgaggcacagaggtacacttacaaagatacaataaatggcagaactaagatttgaacccaggactaagtgtattgcttgtatttatttaattaattaatttttaagagacagggcctcactctgttgcctaggctggcccttgaactcctgggctcaagcagtccacctgcctcagcctcctgagtagctgggactgcaggcacaccatgcctcccagttgtttttaaacactaatagtagtctttcataaggacacttataataaaggcagagctggaacccacacttcattccagactgctcagactgagttagtgttagaaaactgaaagtaacatttttattactgtatttcaggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagtgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttggtcaagactaactttcttttaaaggttcatttgtatgataaattcatatgtgtatatataattttttttgttttgtctagtgagtttcaacatttttaaagttttcaaaaagccatcggaatgttaaattaatgtaaagggaacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagtaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctgttaacttacattgtttaaggtatataatttttaagtcttgcagaatttgtacagcttcgaatgctatagtattagatgctttctttctcatactacatttcttcatttggtttattacattagtctcaccagaccagtacttgcaaaaccctgatggtgctgatcgtagcaggtgatgcatcaccaccaagcatttcaaaaactatttcagccattctaggctagaggcctgagcaagtaaaacatggagaaaaggcagggcagggcctaagaaggatgtgacatgtcctatgataaaggaccaggagtttgggatttgagagagaacaggaagtggtggatagtgaaaatccatctgatgcaaaggctgggttaactgaaaatctgttctgggatgctaggtctttcaagtctgaagactgcagaccacatttggtatcaactttctgaggctgacctgagaacctacttaagccattaaaggttttgagggcgaaagtattactgaagaggccagcagtgtaccccttaaaatgtatgtgtaacttggtcgggtgcagtggctcacgcctgtaatcccagcactctgggaggcctacacgggcggaccacctgaggtcaggagttcaagaccagcctggccaacatggcgaaaccccgtttttactaaaaatacaaaaattagtcgggtgtggtggcggcacctgtagtcccagctatttgggaggctgaggctggagaatcgcttcaacccaggaggtggaggctgcagtgagccaagatcgcgccactgcactccagcctggtgacagagcaagactccgtctcaaagaacaaaaaagtatatgtaatgaataaagctttagcaatttcctgttcccagttctgcctccctccccagcaaactttctaggtagacctgtcctgtatattgttagcactagagaaattacctgtaagatgatatcctcaaaatattacaaaattaaggaacattctatatatgcacaaattgaagaagtcaggtagttgatacttttaaaggtacatagtcaatttaagtggtcaaaaggtatttggcaaaacctaatttaactcaagccctatgcataacgttattgctgaaccactggctaatgcagcatgtactgtatttcactgattgtaataccctaattaatgtcacatggtttaattagcgtttttttttgtagtggtacataaaatgacggtggaccttcaatcaatggtgttttacataatccccaactaagctttatttgaaacacaacttctgacctgtaatctagaaaggggtgggggagtatgtatatgtatgtacaccaaaacctctcccccaaagatctaaagccattctaggccttcagctcaaactaggatactagtagaaagtacagaaaaatggtctaatgacctccccaccttttgaagtgactgtcaaatgagactattaaaaaccacttgaaaacttttaaaaagcaagaatagcctgagacctcctagaaatacagatttaattggtgtaaggtggggtttgtgcagagtattaaaaacgcccccaggcgattcaaaagtatagccagatttgagaaacacccttagctggaaaagaattgaggagctctgttttgatgacttttaacatacactccttagggctggcttaaaaaaggaggttaacttacattagcctcaattcccaaattttacctagaaatgcttcataaattaggtaatttggggttcataagcaaatacagcagagtagggagctatcaaaatagagtgaattcttcagcaaaggaggacaaaactaccataaggcattctggatggtcaaagtgcaaaagaggttggggagtggaggaagagactgatgacaggaatattttgctcttgttttagcaggcttaccatgcactcttcaggggaagactgctctatgccatattacaccttgattcacag

SEQ ID NO: 2 - protein, 20 aa, Artificial Sequence
  Description of Artificial Sequence Synthetic polypeptide
EEKGQSTSSHSKXSTEGKEE

(19) United States
(12) Patent Application Publication
(10) Pub. No.: US 2008/0249103 A1
(43) Pub. Date: Oct. 9, 2008

(54) Sirtuin polymorphisms and methods of use thereof

(75) Inventors: Markku Laakso, Kuopio (FI); Teemu Kuulasmaa, Kuopio (FI); Johan Auwerx, Hindisheim (FR); Christoph Westphal, Brookline, MA (US); Peter Elliott, Marlborough, MA (US); Karl D. Normington, Acton, MA (US); Olivier Boss, Boston, MA (US); Andre Iffland, Cambridge, MA (US); Siva Lavu, Worcester, MA (US)

Correspondence Address: ROPES & GRAY LLP, PATENT DOCKETING 39/41, ONE INTERNATIONAL PLACE, BOSTON, MA, 02110-2624, US

(73) Assignee: Sirtris Pharmaceuticals, Inc., Cambridge, MA (US)

(21) Appl. No.: 11/981,524

(22) Filed: Oct. 30, 2007

Related U.S. Application Data
(60) Provisional application No. 60/859,468, filed on Nov. 15, 2006.

(51) Int. Cl. A61K 31/496; C12Q 1/68; A61K 31/498; A61K 31/429

(52) U.S. Cl. 514/252.11; 435/6; 514/368; 514/254.02

(57) ABSTRACT

Provided herein are methods for diagnosis and prognosis using polymorphic variants of sirtuins. Such polymorphic may be used, for example, to identify subjects that would be responsive to treatment with a sirtuin modulating compound and/or subjects that are suffering from or susceptible to a disease mediated by a sirtuin. Also provided are methods for determining the predictive value of a sirtuin polymorphic variant, methods for evaluating sirtuin modulating compounds, and methods for determining appropriate dosage and/or treatment regimens for subjects having one or more sirtuin polymorphic variants. Screening methods for identifying sirtuin modulating compounds using polymorphic variants of a sirtuin are also provided.

### RELATED APPLICATIONS

This application claims the benefit of U.S. Provisional Application No. 60/859,468, filed Nov. 15, 2006, which application is hereby incorporated by reference in its entirety.

### BACKGROUND

Type 2 Diabetes Mellitus (T2DM) has become a significant epidemic throughout the world. There is a significant unmet medical need for novel mechanism of action therapeutics for the treatment of metabolic diseases such as T2DM. One novel therapeutic approach to treating insulin resistance and T2DM has come from the study of Calorie Restriction (CR), a dietary regimen of consuming 30-40% fewer calories, which has been shown to improve a number of metabolic parameters including insulin sensitivity^{1,2}. The molecular components of the pathway(s) downstream of CR may provide relevant intervention points for the development of therapeutic drugs to treat metabolic disease^{3,4}.

Studies in lower organisms including Saccharomyces cerevisiae and Drosophila melanogaster have led to the identification of key players in these pathways. The protein Sir2 has been identified as one such player that may mediate some of the physiological benefits of CR^{5,6}. In yeast and flies, Sir2 is a histone deacetylase that when overexpressed extends lifespan, and when deleted decreases lifespan^{7,8}. In addition, the ability of CR to extend lifespan in yeast and flies is abrogated when Sir2 is deleted underscoring the importance of this protein in pathways downstream of CR⁸⁻¹⁰. The Sir2 homolog in mammals is SIRT1. Several lines of data support a link between SIRT1 and CR, and suggest a role for this enzyme in mediating some of the health benefits of CR in mammals. SIRT1 levels in several tissues in rodents are increased following a regimen of CR^{11,12}. Resveratrol, a compound that has been shown to induce activation of SIRT1 and mimic the effects of CR in lower organisms, has recently been shown to improve insulin sensitivity, increase mitochondrial content, and survival of mice on a high calorie diet¹³⁻¹⁶.

SIRT1 is a member of the sirtuin family of NAD⁺-dependent deacetylases. These enzymes have evolved to catalyze a unique reaction in which deacetylation of a lysine residue in a substrate protein is coupled to the consumption of NAD^{+17-19.} A number of cellular substrates for SIRT1 have been identified including PGC-1α, NCoR, p300, NFkB, Foxo, and p53²⁰⁻²⁹. Through modulation of the activities of these proteins, SIRT1 regulates mitochondrial biogenesis, metabolism in muscle and adipose tissue, and cellular survival^{11,12,25,30}.

### SUMMARY

In one aspect, the invention provides a method for identifying a subject that would be responsive to treatment with a sirtuin modulating compound, comprising determining the presence or absence of at least one polymorphic variant in a biological sample from said subject, wherein the polymorphic variant is in a nucleic acid sequence that encodes a sirtuin protein or controls expression of a sirtuin gene, and wherein the presence of the at least one polymorphic variant is indicative of a subject that would be responsive to treatment with a sirtuin modulating compound.

In another aspect, the invention provides a method for identifying a subject that would benefit from treatment with a sirtuin modulating compound, comprising determining the presence or absence of at least one polymorphic variant in a biological sample from said subject, wherein the polymorphic variant is in a nucleic acid sequence that encodes a sirtuin protein or controls expression of a sirtuin gene, and wherein the presence of the at least one polymorphic variant is indicative of a subject that would benefit from treatment with a sirtuin modulating compound.

In another aspect, the invention provides a method for evaluating a subject's risk of developing a sirtuin mediated disease or disorder, comprising determining the presence or absence of at least one polymorphic variant in a biological sample from said subject, wherein the polymorphic variant is in a nucleic acid sequence that encodes a sirtuin protein or controls expression of a sirtuin gene, and wherein the presence of the at least one polymorphic variant is indicative of a subject at risk for developing a sirtuin mediated disease or disorder.

In another aspect, the invention provides a method for evaluating a sirtuin modulating compound, comprising: (a) administering a sirtuin modulating compound to a patient population; (b) determining the presence or absence of one or more polymorphic variants of a sirtuin sequence in a biological sample from the patients in said population before or after administering said sirtuin modulating compound to said patient population; (c) evaluating the efficacy of the sirtuin modulating compound in said patient population; and (d) correlating the efficacy of the sirtuin modulating compound with the presence or absence of the one or more polymorphic variants of the sirtuin sequence, thereby evaluating the sirtuin modulator.

In another aspect, the invention provides a method for evaluating a sirtuin modulating compound, comprising: (a) administering a sirtuin modulating compound to a patient population for which the presence or absence of one or more polymorphic variants of a sirtuin sequence has been determined; (b) evaluating the efficacy of the sirtuin modulating compound in said patient population; and (c) correlating the efficacy of the sirtuin modulating compound with the presence or absence of the one or more polymorphic variants of the sirtuin sequence, thereby evaluating the sirtuin modulator.

In another aspect, the invention provides a method for establishing the predictive value of a polymorphic variant of a sirtuin sequence, comprising: (a) determining the presence or absence of one or more polymorphic variants of a sirtuin sequence in a biological sample from patients in a patient population; (b) assaying one or more physiological or metabolic parameters in the patients of said patient population; and (c) correlating the present or absence of the one or more polymorphic variants with the one or more physiological or metabolic parameters in said patient population, wherein a correlation is indicative of the predictive value of the polymorphic variant.

In another aspect, the invention provides a method for treating a sirtuin mediated disease or disorder in a subject, comprising: (a) determining the presence or absence of one or more polymorphic variants in a sirtuin sequence in a biological sample from said subject, thereby producing a polymorphic variant profile for said subject; (b) analyzing the polymorphic variant profile to determine a course of treatment, dosage regimen, or course of treatment and dosage regimen for said subject; and (c) administering a sirtuin modulating compound to said subject according to the determined course of treatment, dosage regimen, or course of treatment and dosage regimen, thereby treating the sirtuin mediated disease or disorder.

In another aspect, the invention provides a method for identifying a sirtuin modulating compound, comprising: (a) contacting a cell comprising a sirtuin sequence having at least one polymorphic variant with a test compound; and (b) determining (i) the level of expression from the sirtuin sequence, (ii) the level of activity of a sirtuin protein expressed by the sirtuin sequence, or (iii) both (i) and (ii), wherein a change in (i), (ii) or both (i) and (ii) in the presence of the test compound as compared to a control is indicative of a compound that is a sirtuin modulating compound.

In another aspect, the invention provides a method for identifying a sirtuin modulating compound, comprising: (a) contacting a cell comprising an expression construct with a test compound, wherein the expression construct comprises a reporter gene operably linked to a sirtuin promoter sequence having at least one polymorphic variant; and (b) determining the level of expression of the reporter gene, wherein a change in the level of expression of the reporter gene in the presence of the test compound as compared to a control is indicative of a compound that is a sirtuin modulating compound.

In certain embodiments, the polymorphic variant is in human SIRT1.

In certain embodiments, the polymorphic variant is selected from the group consisting of: an A variant of single nucleotide polymorphism (SNP) rs3740051, an A variant of SNP rs2236319, or a T variant of SNP rs2273773.

The appended claims are incorporated into this section by reference.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The foregoing and other features and advantages of the present invention will be more fully understood from the following detailed description of illustrative embodiments taken in conjunction with the accompanying drawings in which:

FIG. 1. The association of SNPs of the Sirt1 gene with EE as measured in normal weight offspring of probands with type 2 diabetes (n=123). *LBM, lean body mass, ** EE, energy expenditure mean±SD, p values adjusted for age, gender and familial relationship.

FIG. 2. In vivo activity of SIRT1 activator in a Diet Induced Obesity model. C57BL/6 mice were fed a high fat diet (60% calories from fat) or low fat chow diet until the high fat fed mice reached a weight of 40 g. The mice were dosed once a day by oral gavage with either vehicle, 100 mg/kg SRT1933, or 5 mg/kg rosiglitazone. a, The 100 mg/kg daily oral dose of SRT1933 produced an exposure above the EC_{1.5}=440 nM for at least 16 hrs each day. b, Fed blood glucose and c, glucose excursion during an OGTT improved over the 11 week time course. d, Plasma insulin level and e, insulin sensitivity during an ITT improved over time. f, Body temperature decreased in the SRT1933 treated groups and g, a significant increase in mitochondrial capacity as shown by elevated citrate synthase activity in skeletal muscle with a trend in adipose tissue mice (n=5) after 11 weeks. Data are presented as Vmax per mg protein.

FIG. 3. Diagnostic testing of human clinical samples correlating SNPs of the Sirt1 gene with Sirt1 mRNA, protein level, enzymatic activity and downstream markers with or without pharmacological treatment with Sirt1 modulators.

FIG. 4. Genomic sequence of human Sirt1 gene from GenBank Accession No. DQ278604 (36,853 base pairs) (SEQ ID NO: 1).

FIG. 5. Shows Table 1 providing a variety of polymorphic sites of human Sirt1. The positions are given with reference to the genomic sequence presented in SEQ ID NO: 1.

### DETAILED DESCRIPTION

As described herein, certain regions of the human Sirt1 genomic DNA (chromosomal locations) harbor mutations or variations that could contribute or be predictive of the development of diseases and disorders including, for example, diseases or disorders related to aging or stress, diabetes, obesity, neurodegenerative diseases, diseases or disorders associated with mitochondrial dysfunction, chemotherapeutic induced neuropathy, neuropathy associated with an ischemic event, ocular diseases and/or disorders, cardiovascular disease, blood clotting disorders, inflammation, and/or flushing, etc. (herein referred to as disease or diseases in general). These regions, or susceptibility loci, are typically on the order of many kilobases or megabases in length and mutations or alterations somewhere within these regions are believed to confer an increased likelihood that an individual having such mutations or alterations will develop the condition. It is therefore likely that such regions harbor genes which, alone or in combination, are causally implicated in disease in at least a subset of patients. Genetic studies include linkage studies, in which families having an increased incidence of disease relative to the incidence in the general population, e.g., disease families, and association studies, in which populations typically containing both related and unrelated subjects diagnosed with disease, e.g., groups of disease families, are studied. Association studies can compare the frequencies of certain haplotypes in control and affected populations. Alternately, they can assess disequilibrium in the transmission of certain haplotypes to affected probands.

Linkage and association studies typically make use of genetic polymorphisms. (See, e.g., Cardon, L. and Bell, J., (2001), Nature Reviews Genetics, Vol. 2, pp. 91-99; Kruglyak, L. and Lander, E. (1995), Am. J. Hum. Genet., 56:1212-1223; Jorde, L. B. (2000), Genome Research, 10:1435-1444; Pritchard, J. and Przeworski, M. (2001), Am. J. Hum. Genet., 69:1-14 and references in the foregoing articles for discussion of considerations in design of genetic studies). For example, a population may contain multiple subpopulations of individuals each of which has a different DNA sequence at a particular chromosomal location.

It will be appreciated that while certain polymorphic variants may be responsible for disease or phenotypic variation by, for example, causing a functional alteration in an encoded protein, many polymorphisms appear to be silent in that no known detectable difference in phenotype exists between individuals having different alleles. However, polymorphisms (whether silent or not) may be physically and/or genetically linked to genes or DNA sequences in which mutations or variations confer susceptibility to and/or play a causative role in disease (i.e., they are located within a contiguous piece of DNA). In the absence of genetic recombination, polymorphisms that are physically linked to such mutations or variations will generally be inherited together with the mutation or alteration.

With increasing genetic recombination between any given polymorphism and a causative mutation or variation, the extent of co-inheritance will be reduced. Since the likelihood of genetic recombination between loci generally increases with increasing distance between the loci (though not necessarily in a linear fashion), co-inheritance of a particular polymorphism and a particular phenotype suggests that the polymorphism is located in proximity to a causative mutation or variation. Thus studying the co-inheritance of polymorphic variants, e.g., SNPs, allows identification of genomic regions likely to harbor a mutation or variation that, alone or in combination with other mutations or variations, causes or increases susceptibility to disease. Polymorphisms are thus useful for genetic mapping and identification of candidate genes, in which mutations or variations may play a causative role in disease. In addition, detection of particular polymorphic variants (alleles) is useful for diagnosis of disease or susceptibility to disease as described herein.

1. Polymorphic Variants Associated with Sirtuin Mediated Diseases and Disorders

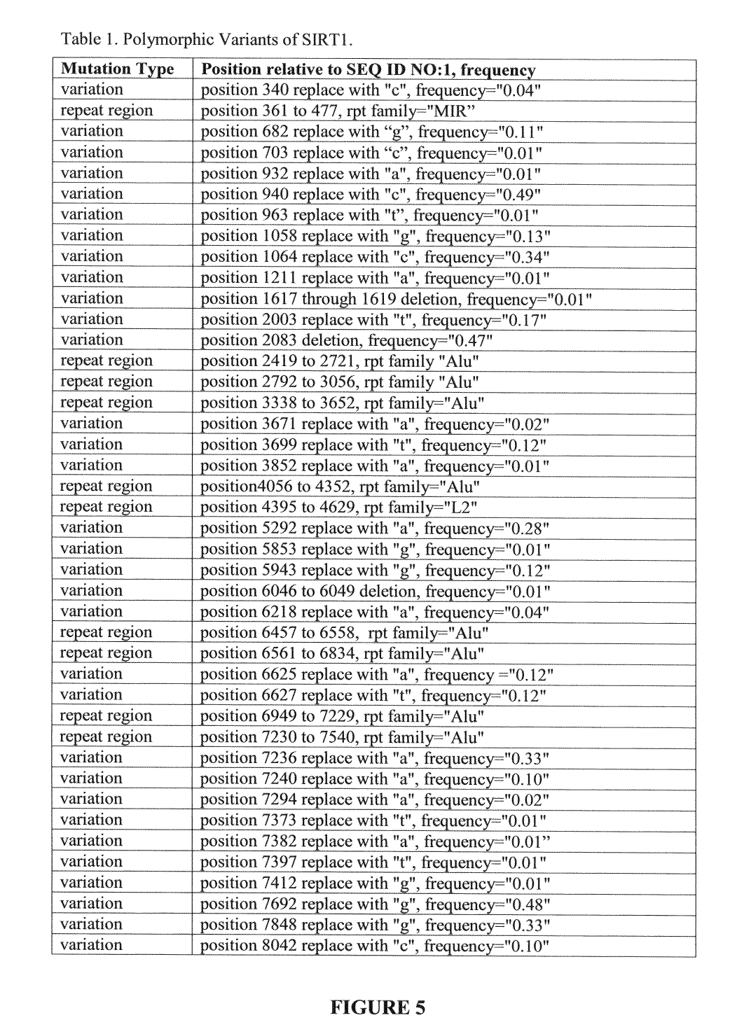

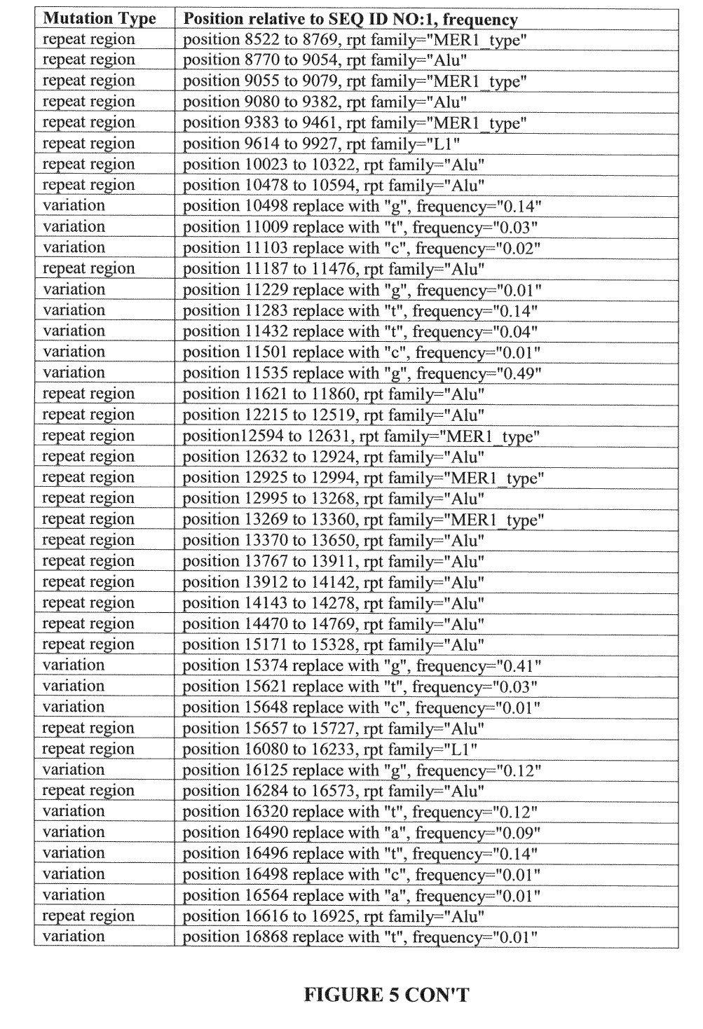

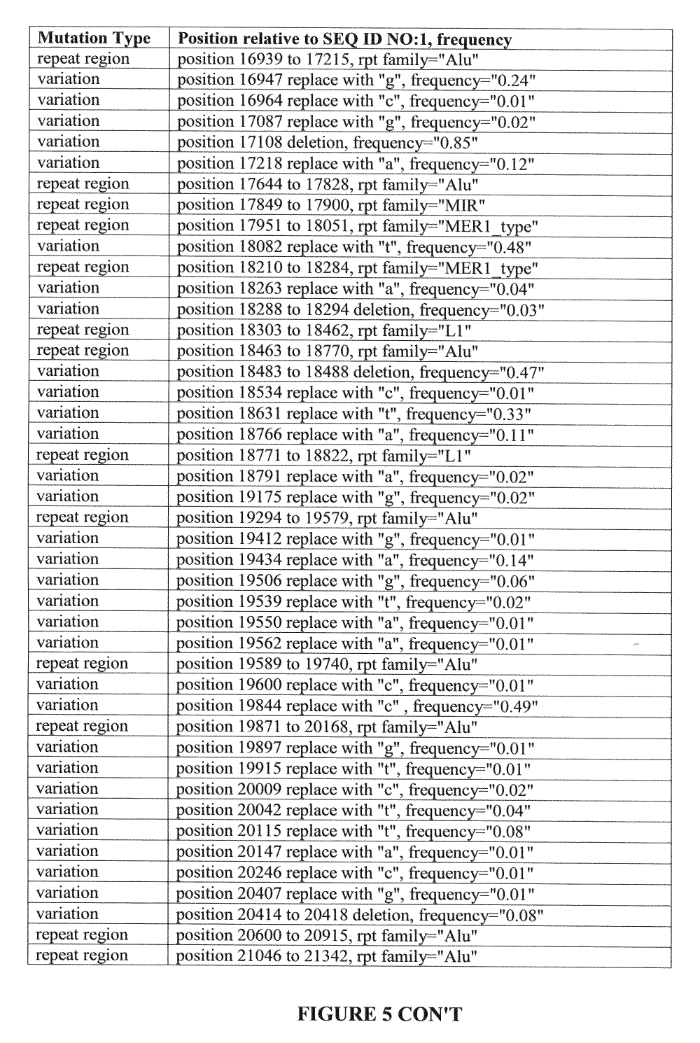

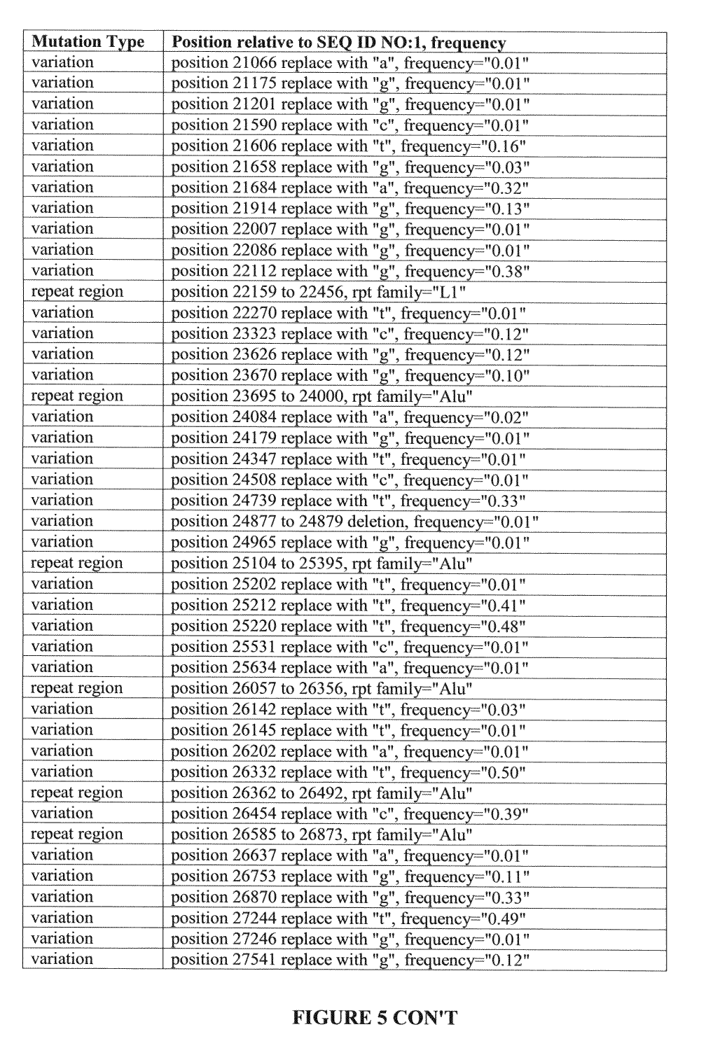

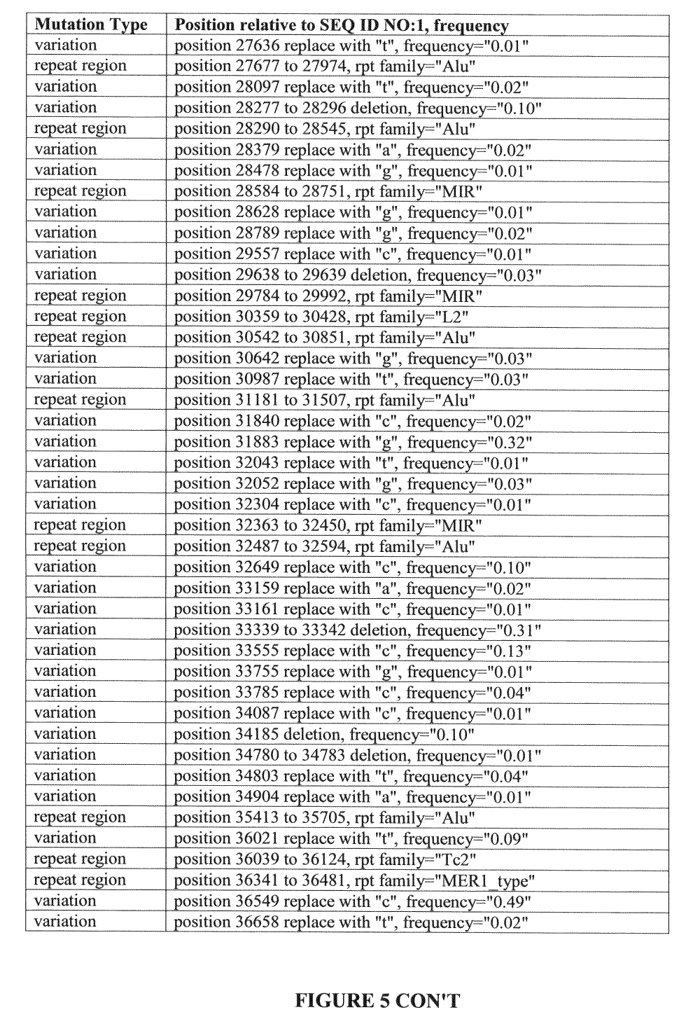

Provided herein are polymorphic variants of Sirt1 that are associated with a sirtuin mediated diseases or disorders. Exemplary polymorphic variants of Sirt1 are shown in Table 1 (see FIG. 5). Other polymorphic variants of Sirt1 include rs12778366, rs3740051, rs2236319, rs2272773, and rs10997870. Yet other polymorphic variants of Sirt1 include rs730821, rs3084650, rs4746715, rs4745944, rs3758391, rs3740051, rs932658, rs3740053, rs2394443, rs932657, rs737477, rs911738, rs4351720, rs2236318, rs2236319, rs768471, rs1885472, rs2894057, rs4746717, rs2224573, rs2273773, rs3818292, rs1063111, rs1063112, rs1063113, rs1063114, rs3818291, rs5785840, rs2394444, rs1467568, rs1966188, rs2394445, rs2394446, i-s4746720, rs752578, rs2234975, rs1022764, rs1570290, rs2025162, rs4141919, rs14819, and rs14840 (see e.g., WO2005/004814). See e.g., the Entrez SNP database operated by the National Center for Biotechnology Information (NCBI) on the world wide web at ncbi.nlm.nih.gov/entrez/query.fcgi?CMD=search&DB=snp.

In an exemplary embodiment, polymorphic variants of Sirt1 include an A variant of SNP rs3740051 (e.g., an A at position 682 in SEQ ID NO: 1), an A variant of SNP rs2236319 (e.g., an A at position 5943 in SEQ ID NO: 1), and a T variant of SNP rs2273773 (e.g., a T at position 23,323 in SEQ ID NO: 1).

Studies provided herein have linked insuring sensitivity and increased energy expenditure with polymorphic variants of SIRT1. Insuling sensitivity and energy expenditure have been demonstrated to be effects of administration of sirtuin activating compounds in a diet induced obesity model in mice along with normalized glucose and insulin levels, elevated mitochondrial function and lower core body temperature (see e.g., PCT Application No. PCT/US06/026272 which is herein incorporated by reference in its entirety). As used herein, the term “polymorphic site” refers to a region in a nucleic acid at which two or more alternative nucleotide sequences are observed in a significant number of nucleic acid samples from a population of individuals. A polymorphic site may be a nucleotide sequence of two or more nucleotides, an inserted nucleotide or nucleotide sequence, a deleted nucleotide or nucleotide sequence, or a microsatellite, for example. A polymorphic site that is two or more nucleotides in length may be 3, 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region. A polymorphic site is often one nucleotide in length, which is referred to herein as a “single nucleotide polymorphism” or a “SNP.”

Where there are two, three, or four alternative nucleotide sequences at a polymorphic site, each nucleotide sequence is referred to as a “polymorphic variant” or “nucleic acid variant.” Where two polymorphic variants exist, for example, the polymorphic variant represented in a minority of samples from a population is sometimes referred to as a “minor allele” and the polymorphic variant that is more prevalently represented is sometimes referred to as a “major allele.” Many organisms possess a copy of each chromosome (e.g., humans), and those individuals who possess two major alleles or two minor alleles are often referred to as being “homozygous” with respect to the polymorphism, and those individuals who possess one major allele and one minor allele are normally referred to as being “heterozygous” with respect to the polymorphism. Individuals who are homozygous with respect to one allele are sometimes predisposed to a different phenotype as compared to individuals who are heterozygous or homozygous with respect to another allele.

In the genetic studies presented herein that associate insulin sensitivity and energy expenditure with polymorphic variants of SIRT1, samples from healthy, normal weight, non-diabetic offspring of type 2 diabetic patients were allelotyped and genotyped. The term “genotyped” as used herein refers to a process for determining a genotype of one or more individuals, where a “genotype” is a representation of one or more polymorphic variants in a population. Genotypes may be expressed in terms of a “haplotype,” which as used herein refers to two or more polymorphic variants occurring within genomic DNA in a group of individuals within a population. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

As used herein, the term.“phenotype” refers to a trait which can be compared between individuals, such as presence or absence of a condition, a visually observable difference in appearance between individuals, metabolic variations, physiological variations, variations in the function of biological molecules, and the like. An example of a phenotype is occurrence of breast cancer.

Researchers sometimes report a polymorphic variant in a database without determining whether the variant is represented in a significant fraction of a population. Because a subset of these reported polymorphic variants are not represented in a statistically significant portion of the population, some of them are sequencing errors and/or not biologically relevant. Thus, it is often not known whether a reported polymorphic variant is statistically significant or biologically relevant until the presence of the variant is detected in a population of individuals and the frequency of the variant is determined. Methods for detecting a polymorphic variant in a population are described herein. A polymorphic variant is statistically significant and often biologically relevant if it is represented in 5% or more of a population, sometimes 10% or more, 15% or more, or 20% or more of a population, and often 25% or more, 30% or more, 35% or more, 40% or more, 45% or more, or 50% or more of a population.

A polymorphic variant may be detected on either or both strands of a double-stranded nucleic acid. For example, a thymine at a particular position in SEQ ID NO: 1 can be reported as an adenine from the complementary strand. Also, a polymorphic variant may be located within an intron or exon of a gene or within a portion of a regulatory region such as a promoter, a 5′ untranslated region (UTR), a 3′ UTR, and in DNA (e.g., genornic DNA (gDNA) and complementary DNA (cDNA)), RNA (e.g., mRNA, tRNA, and rRNA), or a polypeptide. Polymorphic variations may or may not result in detectable differences in gene expression (mRNA and/or protein expression), polypeptide structure, polypeptide sequence, or polypeptide function. Preferred polymorphic variations of Sirt1 do result in detectable differences in gene expression (mRNA and/or protein expression), polypeptide structure, polypeptide sequence, or polypeptide function.

2. Additional Polymorphic Variants Associated with Sirtuin Mediated Diseases and Disorders

Also provided are methods for identifying polymorphic variants proximal to an incident, founder polymorphic variant associated with a sirtuin mediated disease or disorder. Thus, featured herein are methods for identifying a polymorphic variation associated with sirtuin mediated diseases and disorder that is proximal to an incident polymorphic variation associated with a sirtuin mediated disease or disorder, which comprises identifying a polymorphic variant proximal to the incident polymorphic variant associated with a sirtuin mediated disease or disorder, where the incident polymorphic variant is in a sirtuin gene or regulatory sequence. The presence or absence of an association of the proximal polymorphic variant with sirtuin mediated diseases and disorders then is determined using a known association method, such as a method described herein. In one embodiment, the incident polymorphic variant is present in a sirtuin gene or regulatory sequence. The proximal polymorphic variant identified may be a publicly disclosed polymorphic variant, which for example, sometimes is published in a publicly available database. Altemativley, the polymorphic variant identified is not publicly disclosed and is discovered using a known method, including, but not limited to, sequencing a region surrounding the incident polymorphic variant in a group of nucleic acid samples. Thus, multiple polymorphic variants proximal to an incident polymorphic variant are associated with a sirtuin mediated disease or disorder using this method.

The proximal polymorphic variant often is identified in a region surrounding the incident polymorphic variant. In certain embodiments, this surrounding region is about 50 kb flanking the first polymorphic variant (e.g. about 50 kb 5′ of the first polymorphic variant and about 50 kb 3′ of the first polymorphic variant), and the region sometimes is composed of shorter flanking sequences, such as flanking sequences of about 40 kb, about 30 kb, about 25 kb, about 20 kb, about 15 kb, about 10 kb, about 7 kb, about 5 kb, or about 2 kb 5′ and 3′ of the incident polymorphic variant. In other embodiments, the region is composed of longer flanking sequences, such as flanking sequences of about 55 kb, about 60 kb, about 65 kb, about 70 kb, about 75 kb, about 80 kb, about 85 kb, about 90 kb, about 95 kb, or about 100 kb 5′ and 3′ of the incident polymorphic variant.

In certain embodiments, polymorphic variants associated with a sirtuin mediated disease or disorder are identified iteratively. For example, a first proximal polymorphic variant is associated with a sirtuin mediated disease or disorder using the methods described herein and then another polymorphic variant proximal to the first proximal polymorphic variant is identified (e.g., publicly disclosed or discovered) and the presence or absence of an association of one or more other polymorphic variants proximal to the first proximal polymorphic variant with a sirtuin mediated disease or disorder is determined.

The methods described herein are useful for identifying or discovering additional polymorphic variants that may be used to further characterize a gene, region or loci associated with a a sirtuin mediated disease or disorder. For example, allelotyping or genotyping data from the additional polymorphic variants may be used to identify a functional mutation or a region of linkage disequilibrium.

In certain embodiments, polymorphic variants identified or discovered within a region comprising the first polymorphic variant associated with a sirtuin mediated disease or disorder are genotyped using the genetic methods and sample selection techniques described herein, and it can be determined whether those polymorphic variants are in linkage disequilibrium with the first polymorphic variant. The size of the region in linkage disequilibrium with the first polymorphic variant also can be assessed using these genotyping methods. Thus, provided herein are methods for determining whether a polymorphic variant is in linkage disequilibrium with a first polymorphic variant associated with a sirtuin mediated disease or disorder, and such information can be used in prognosis methods described herein.

### 3. Methods for Detecting Polymorphic Variants

In certain embodiments, the methods described herein involve determining the presence or absence of a polymorphic variant of a sirtuin gene, such as Sirt1, in a subject or patient population. Any method for determining the presence or absence of a polymorphic variant may be used in accordance with the methods described herein. Such methods include, for example, detection of a polymorphic variant in a nucleic acid sequence such as genomic DNA, cDNA, mRNA, tRNA, rRNA, etc. Variants may be located in any region of a nucleic acid sequence including coding regions, exons, introns, intron/exon borders and regulatory regions, such as promoters, enchancers, termination sequences, etc. Certain polymorphic variants may be associated with differences in gene expression (mRNA and/or protein), post-transcriptional regulation and/or protein activity. For such polymorphic variants, determining the presence or absence of the polymorphic variant may involve determining the level of transcription, mRNA maturation, splicing, translation, protein level, protein stability, and/or protein activity. Polymorphic variants that lead to a change in protein sequence may also be determined by identifying a change in protein sequence and/or structure. A variety of methods for detecting and identifying polymorphic variants are known in the art and are described herein.

Polymorphic variants may be detected in a subject using a biological sample from said patient. Various types of biological samples may be used to detect the presence or absence of a polymorphic variant in said subject, such as, for example, samples of blood, serum, urine, saliva, cells (including cell lysates), tissue, hair, etc. Biological samples suitable for use in accordance with the methods described herein will comprise a Sirt1 nucleic acid or polypeptide sequence. Biological samples may be obtained using known techniques such as venipuncture to obtain blood samples or biopsies to obtain cell or tissue samples.

Diagnostic procedures may also be performed in situ directly upon tissue sections (fixed and/or frozen) obtained from a patient such that no nucleic acid purification is necessary. Nucleic acids may be used as probes and/or primers for such in situ procedures (see, for example, Nuovo, G. J., 1992, PCR in situ hybridization: protocols and applications, Raven Press, New York).

The methods described herein may be used to determine the genotype of a subject with respect to both copies of the polymorphic site present in the genome. For example, the complete genotype may be characterized as −/− , as ±, or as +/+, where a minus sign indicates the presence of the reference sequence at the polymorphic site, and the plus sign indicates the presence of a polymorphic variant other than the reference sequence. If multiple polymorphic variants exist at a site, this can be appropriately indicated by specifying which ones are present in the subject. Any of the detection means described herein may be used to determine the genotype of a subject with respect to one or both copies of the polymorphism present in the subject's genome.

According to certain embodiments of the invention it is preferable to employ methods that can detect the presence of multiple polymorphic variants (e.g., polymorphic variants at a plurality of polymorphic sites) in parallel or substantially simultaneously. Oligonucleotide arrays represent one suitable means for doing so. Other methods, including methods in which reactions (e.g., amplification, hybridization) are performed in individual vessels, e.g., within individual wells of a multi-well plate or other vessel may also be performed so as to detect the presence of multiple polymorphic variants (e.g., polymorphic variants at a plurality of polymorphic sites) in parallel or substantially simultaneously according to certain embodiments of the invention.

Examples of techniques for detecting differences of at least one nucleotide between two nucleic acids include, but are not limited to, selective oligonucleotide hybridization, selective amplification, or selective primer extension.

A preferred detection method is allele specific hybridization using probes overlapping the polymorphic site and having about 5, 10, 20, 25, or 30 nucleotides around the polymorphic site. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl. Acad. Sci USA 86:6230; and Wallace et al. (1979) Nucl. Acids Res. 6:3543). Such allele specific oligonucleotide hybridization techniques may be used for the simultaneous detection of several nucleotide changes in different polymorphic regions of gene. Examples of probes for detecting specific polymorphic variants of the polymorphic site located in the Sirt1 gene are probes comprising about 5, 10, 20, 25, 30, 50, 75 or 100 nucleotides of SEQ ID NO: 1 or about 5, 10, 20, 25, 30, 50, 75 or 100 nucleotides of a sequence complmentary to SEQ ID NO: 1. In one embodiment, oligonucleotides having nucleotide sequences of specific polymorphic variants are attached to a hybridizing membrane and this membrane is then hybridized with labeled sample nucleic acid. Analysis of the hybridization signal will then reveal the identity of the polymorphic variants of the sample nucleic acid. In a preferred embodiment, several probes capable of hybridizing specifically to polymorphic variants are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244 and in Kozal et al. (1996) Nature Medicine 2:753. In one embodiment, a chip comprises all the polymorphic variants of at least one polymorphic region of a gene. The solid phase support is then contacted with a test nucleic acid and hybridization to the specific probes is detected. Accordingly, the identity of numerous polymorphic variants of one or more genes can be identified in a simple hybridization experiment. For example, the identity of the polymorphic variant at any of the polymorphic sites described herein can be determined in a single hybridization experiment. In an exemplary embodiment, the identify of the polymorphic variant at the five SNPs rs12778366, rs3740051, rs2236319, rs2273773, and rs10997870 maybe determined in a single hybridization experiment.

Alternatively, allele specific amplification technology which depends on selective PCR amplification may be used. Oligonucleotides used as primers for specific amplification may carry the polymorphic variant of interest in the center of the molecule (so that amplification depends on differential hybridization) (Gibbs et al (1989) Nucleic Acids Res. 17:2437-2448) or at the extreme 3′ end of one primer where, under appropriate conditions, a mismatch can prevent or reduce polymerase extension (Prossner (1993) Tibtech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al (1992) Mol. Cell Probes 6:1).

Various dection methods described herein involve first amplifying at least a portion of a gene prior to identifying the polymorphic variant. Amplification can be performed, e.g., by PCR and/or LCR, according to methods known in the art. In one embodiment, genomic DNA of a cell is exposed to two PCR primers and amplification is carried out for a number of cycles that is sufficient to produce the required amount of amplified DNA. The primers may be about 5-50, about 10-50, about 10-40, about 10-30, about 10-25, about 15-50, about 15-40, about 15-30, about 15-25, or about 25-50 nucleotides in length and may be designed to hybridize to sites about 40-500 base pairs apart (e.g., to amplify a nucleotide sequence of about 40-500 base paris in length).

Additional amplification methods include, for example, self sustained sequence replication (Guatelli, J. C. et al., 1990, Proc. Natl. Acad. Sci. U.S.A. 87:1874-1878), transcriptional amplification system (Kwoh, D. Y. et al., 1989, Proc. Natl. Acad. Sci. U.S.A. 86:1173-1177), Q-Beta Replicase (Lizardi, P. M. et al., 1988, Bio/Technology 6:1197), or any other nucleic acid amplification method, followed by the detection of the amplified molecules using techniques well known to those of skill in the art. These detection schemes are especially useful for the detection of nucleic acid molecules that may be present in very low numbers.

Any of a variety of sequencing reactions known in the art can be used to directly sequence at least a portion of a gene and detect polymorphic variants by comparing the sequence of the sample sequence with the corresponding control sequence. Exemplary sequencing reactions include those based on techniques developed by Maxam and Gilbert (Proc. Natl. Acad Sci USA (1977) 74:560) or Sanger (Sanger et al (1977) Proc. Nat. Acad. Sci 74:5463). It is also contemplated that any of a variety of automated sequencing procedures may be utilized to identify polymorphic variants (Biotechniques (1995) 19:448), including sequencing by mass spectrometry. See, for example, U.S. Pat. No. 5,547,835 and international patent application Publication Number WO 94/16101, entitled DNA Sequencing by Mass Spectrometry by H. Koster; U.S. Pat. No. 5,547,835 and international patent application Publication Number WO 94121822 entitled “DNA Sequencing by Mass Spectrometry Via Exonuclease Degradation” by H. Koster, and U.S. Pat. No. 5,605,798 and International Patent Application No. PCT/US96/03651 entitled DNA Diagnostics Based on Mass Spectrometry by H. Koster; Cohen et al. (1996) Adv Chromatogr 36:127-162; and Griffin et al. (1993) Appl Biochem Biotechnol 38:147-159. It will be evident to one skilled in the art that, for certain embodiments, the occurrence of only one, two or three of the nucleic acid bases need be determined in the sequencing reaction. For instance, for a single nucleotide run, such as an A-track, only one nucleotide needs to be detected and therefore modified seuqencing reactions can be carried out.

Yet other suitable sequencing methods are disclosed, for example, in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”.

In some cases, the presence of a specific polymorphic variant in a DNA sample from a subject can be shown by restriction enzyme analysis. For example, a specific polymorphic variant can result in a nucleotide sequence comprising a restriction site which is absent from a nucleotide sequence of another polymorphic variant.

In other embodiments, alterations in electrophoretic mobility may be used to identify the polymorphic variant. For example, single strand conformation polymorphism (SSCP) may be used to detect differences in electrophoretic mobility between polymorphic variants (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence and the resulting alteration in electrophoretic mobility enables the detection of even a single base change. The DNA fragments may be labeled or detected with labeled probes. The sensitivity of the assay may be enhanced using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (see e.g., Keen et al. (1991) Trends Genet 7:5).

In yet another embodiment, the identity of a polymorphic variant of a may be obtained by analyzing the movement of a nucleic acid comprising the polymorphic variant in polyacrylamide gels containing a gradient of denaturant, e.g., denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In other embodiments, a temperature gradient may be used in place of a denaturing agent gradient to identify differences in the mobility of control and sample DNA (Rosenbaum and Reissner (1987) Biophys Chem 265:1275).

In another embodiment, identification of the polymorphic variant is carried out using an oligonucleotide ligation assay (OLA), as described, e.g., in U.S. Pat. No. 4,998,617 and in Landegren, U. et al., Science 241:1077-1080 (1988). The OLA protocol uses two oligonucleotides which are designed to be capable of hybridizing to abutting sequences of a single strand of a target. One of the oligonucleotides is linked to a separation marker, e.g, biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using a biotin ligand, such as avidin. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990). In this method, PCR is used to achieve the exponential amplification of target DNA which is then detected using OLA.

Several techniques based on this OLA method have been developed and can be used to detect specific polymorphic variants of a gene. For example, U.S. Pat. No. 5,593,826 discloses an OLA using an oligonucleotide having 3′—amino group and a 5′—phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected using hapten specific antibodies that are differently labeled, for example, with enzyme reporters such as alkaline phosphatase or horseradish peroxidase. This system permits the detection of the two alleles using a high throughput format that leads to the production of two different colors.

Polymorphic variants may also be identified using methods for detecting single nucleotide polymorphisms. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C.R. (U.S. Pat. No. 4,656,127). According to the method, a primer complementary to the allelic sequence immediately 3′ to the polymorphic site is permitted to hybridize to a target molecule obtained from a subject. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data

In another embodiment of the invention, a solution-based method is used for determining the identity of a polymorphic variant. Cohen, D. et al. (French Patent 2,650,840; PCT Publication No. WO 91/02087). As in the Mundy method of U.S. Pat. No. 4,656,127, a primer is employed that is complementary to allelic sequences immediately 3′ to a polymorphic site. The method determines the identity of the nucleotide at that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet et al. (PCT Publication No. WO 92/15712). The method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990), Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBAT in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

If a polymorphic variant is located in an exon (either a coding or non-coding exon), the identity of-the polymorphic variant can be determined by analyzing the molecular structure of the mRNA, pre-mRNA, or cDNA. The molecular structure can be determined using any of the above described methods for determining the molecular structure of the genomic DNA, e.g., sequencing and SSCP. In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

Additional methods may be used for determining the identity of a polymorphic variant located in the coding region of a gene. For example, identification of a polymorphic variant which encodes a protein having a sequence variation can be performed using an antibody that specifical recognizes the protein variant, for example, using immunohistochemistry, immunoprecipitation or immunoblotting techniques. Antibodies to protein variants may be prepared according to methods known in the art and as described herein.

In certain embodiments, polymorphic variants may be detected by determining variations in sirtuin protein expression and/or activity. The expression level (i.e., abundance), expression pattern (e.g., temporal or spatial expression pattern, which includes subcellular localization, cell type specificity), size, sequence, association with other cellular constituents (e.g., in a complex such as a SIRT1 complex), etc., of SIRT1 in a sample obtained from a subject may be determined and compared with a control, e.g., the expression level or expression pattern that would be expected in a sample obtained from a normal subject.

In general, such detection and/or comparison may be performed using any of a number of suitable methods known in the art including, but not limited to, immunoblotting (Western blotting), immunohistochemistry, ELISA, radioimmunoassay, protein chips (e.g., comprising antibodies to the relevant proteins), etc. Historical data (e.g., the known expression level, activity, expression pattern, or size in the normal population) may be used for purposes of the comparison. Such methods may utilize SIRT1 antibodies that can distinguish between SIRT1 variants that differ at sites encoded by polymorphic variants.

Generally applicable methods for producing antibodies are well known in the art and are described extensively in references cited above, e.g., Current Protocols in Immunology and Using Antibodies: A Laboratory Manual. It is noted that antibodies can be generated by immunizing animals (or humans) either with a full length polypeptide, a partial polypeptide, fusion protein, or peptide (which may be conjugated with another moiety to enhance immunogenicity). The specificity of the antibody will vary depending upon the particular preparation used to immunize the animal and on whether the antibody is polyclonal or monoclonal. For example, if a peptide is used the resulting antibody will bind only to the antigenic determinant represented by that peptide. It may be desirable to develop and/or select antibodies that specifically bind to particular regions of SIRT1. Such specificity may be achieved by immunizing the animal with peptides or polypeptide fragments that correspond to the desired region or SIRT1. Alternately, a panel of monoclonal antibodies can be screened to identify those that specifically bind to the desired region of SIRT1. Antibodies that specifically bind to antigenic determinants that comprise a region encoded by a polymorphic site of SIRT1 are useful in accordance with the methods described herein. According to certain embodiments, such antibodies are able to distinguish between SIRT1 polypeptides that differ by a single amino acid. Any of the antibodies described herein may be labeled. The methods described herein may also utilize panels of antibodies able to specifically bind to a variety of polymorphic variants of Sirt1.

In general, preferred antibodies will possess high affinity, e.g., a K_{d} of <200 nM, and preferably, of <100 nM for a specific polymorphic variant of SIRT1. Exemplary antibodies do not show significant reactivity (e.g., less than about 50%, 25%, 10%, 5%, 1%, or less, cross reactivity) with a different Sirt1 polymorphic variant.

In other embodiments, polymorphic variants may be determined by determining a change in level of activity of a SIRT1 protein. Such activity may be measured in a biological sample obtained from a subject. Methods for measuring SIRT1 activity, e.g., deacetylase activity, are known in the art and are further described in the Exemplification section herein.

### 4. Methods of Diagnosis and Prognosis

Provided herein are methods for diagnosis and prognosis of sirtuin mediated diseases and disorders, particularly Sirt1 mediated diseases and disorders, using one or more polymorphic variants of Sirt1. The methods disclosed herein may be used, for example, to identify a subject suffering from or susceptible to a sirtuin mediated disease or disorder, to identify a subject that would benefit from treatment with a sirtuin modulating compound, to predict the immediacy of onset and/or severity of a sirtuin mediated disease or disorder, to evaluate a subject's risk of devleoping a sirtuin mediated disease or disorder, to determine appropriate dosage and/or treatment regimens for subjects having one or more Sirt1 polymorphic variants, to determine the responsiveness of an individual with a sirtuin mediated disease or disorder to treatment with a sirtuin modulating compound, and/or to design individualized therapeutic treatments based on the presence or absence of one or more polymorphic variants in a subject.

A sirtuin mediated disease or disorder refer to a disease, disorder or condition that is associated with a change in the level and/or activity of a sirtuin protein. A Sirt1 mediated disease or disorder refer to a disease, disorder or condition that is associated with a change in the level and/or activity of a SIRT1 protein. Examples of sirtuin or Sirt1 mediated diseases or disorders that involve a level of sirtuin or Sirt1 expression and/or activity that is lower than desired include, for example, aging, stress, diabetes, obesity, neurodegenerative diseases, chemotherapeutic induced neuropathy, neuropathy associated with an ischemic event, ocular diseases and/or disorders, cardiovascular disease, blood clotting disorders, inflammation, flushing, disease associated with abnormal mitochondrial activity, decreased muscle performance, decreased muscle ATP levels, or muscle tissue damage associated with hypoxia or ischemia. Examples of sirtuin or Sirt1 mediated diseases or disorders that involve a level of sirtuin or SIRT1 expression and/or activity that is higher than desired include, for example, cancer, suppressed appetite, and/or anorexia.

A sirtuin protein refers to a member of the sirtuin deacetylase protein family, or preferably to the sir2 family, which include human SIRT1 (GenBank Accession No. NM_{—}012238 and NP_{—}036370 (or AF083106)), SIRT2 (GenBank Accession No. NM_{—}012237, NM_{—}030593, NP_{—}036369, NP_{—}085096, and AF083107), SIRT3, SIRT4, SIRT5, SIRT6 and SIRT7 (Brachmann et al. (1995) Genes Dev. 9:2888 and Frye et al. (1999) BBRC 260:273) proteins.

The distribution of one or more Sirt1 polymorphic variants in a large number of individuals exhibiting particular markers of disease status or drug response may be determined by any of the methods described above and compared with the distribution of polymorphic variants in patients that have been matched for age, ethnic origin, and/or any other statistically or medically relevant parameters, who exhibit quantitatively or qualitatively different status markers. Correlations are achieved using any method known in the art, including nominal logistic regression, chi square tests or standard least squares regression analysis. In this manner, it is possible to establish statistically significant correlations between particular polymorphic variants and particular disease statuses (given in p values). It is further possible to establish statistically significant correlations between particular polymorphic variants and changes in disease status or drug response such as would result, e.g., from particular treatment regimens. In this manner, it is possible to correlate polymorphic variants with responsivity to particular treatments.

In certain embodiments, a panel of polymorphic variants may be defined that predict the risk of a sirtuin mediated disease or disorder and/or predict drug response to a sirtuin modulating compound. This predictive panel is then used for genotyping of patients on a platform that can genotype multiple polymorphic variants, such as SNPs, at the same time (Multiplexing). Preferred platforms include, for example, gene chips (Affymetrix) or the Luminex LabMAP reader. The subsequent identification and evaluation of a patient's haplotype can then help to guide specific and individualized therapy.

For example, the methods disclosed herein permit the identification of patients exhibiting polymorphic variants that are associated with an increased risk for adverse drug reactions (ADR). In such cases, dose of a sirtuin modulating compound can be lowered to reduce or eliminate the risk for ADR. Also if a patient's response to drug administration is particularly high (e.g., the patient does not metabolize the drug well), the dose of the sirtuin modulating compound can be lowered to avoid the risk of ADR. In turn if the patient's response to drug administration is low (e.g., the patient is a particularly high metabolizer of the drug), and there is no evident risk of ADR, the dose of the sirtuin modulating compound can be raised to an efficacious level.

The ability to predict a patient's individual drug response to a sirtuin modulating compound permits formulation of sirtuin modulating compounds to be tailored in a way that suits the individual needs of the patient or class of patinets (e.g., low/high responders, poor/good metabolizers, ADR prone patients, etc.). For example, formulations of sirtuin modulating compounds may be individualized to encompass different sirtuin modualting compounds, different doses of the drug, different modes of administration, different frequencies of administration, and different pharmaceutically acceptable carriers. The individualized sirtuin modulating formulation may also contain additional substances that facilitate the beneficial effects and/or diminish the risk for ADR (Folkers et al. 1991, U.S. Pat. No. 5,316,765).

The present invention also provides a business method for determining whether a subject has a sirtuin mediated disease or disorder or a pre-disposition to a sirtuin mediated disease or disorder. Such methods may comprise, for example, obtaining information about the presence or absence of one or more polymorphic variants of Sirt1 for said subject. Other information such as phenotypic information about said subject may also be obtained. This information may then be analyzed to correlate the one or more polymorphic variants of Sirt1 with a risk of developing a sirtuin mediated disease or disorder, severity of a sirtuin mediated disease or disorder, optimal therapeutic treatments, dosage schedules, etc. The method may further comprise the step of recommending a particular treatment for treating or preventing the sirtuin mediated disease or disorder.

In another embodiment, the invention provides a method for pedicting the lifespan of an individual. The method comprises determining the presence or absence of one or more polymorphic variants of Sirt1 in a subject and using the information to calculate a predicted lifespan for said individual. Additional information, such as, one or more additional lifespan factors including age, gender, weight, smoking, disease, etc. may be used in conjunction with the Sirt1 haplotype to calculate the predicted lifespan. Such information can be used, for example, in association with pricing and issuance of insurance policies such as life insurance policies.

In another embodiment, the invention provides a method for evaluating stem cells to be used in association with various cell therapies and methods of treatment using such stem cells. For example, stem cells having a more favorable Sirt1 haplotype may be selected over stem cells having a less favorable Sirt1 haplotype for cell therapy. Stem cells include any type of stem cells suitable for cell therapy including embryonic stem cells. Such stem cells may be used for treating a variety of diseases and disorders including, for example, Parkinson's disease, Huntington's disease and Alzheimer's disease. Exemplary methods may comprise, for example: identifying the presence or absence of one or more polymorphic variants in one or more stem cell samples, identifying a stem cell sample having a favorable Sirt1 haplotype, and using the identified population of stem cells in association with cell therapy for treatment of a disease or disorder that would benefit from the cell therapy.

### 5. Pharmacogenetic and Pharmacogenomic Uses

In various embodiments, knowledge of polymorphisms can be used to help identify patients most suited to therapy with particular pharmaceutical agents (this is often termed “pharmacogenetics”). Pharmacogenetics can also be used in pharmaceutical research to assist the drug selection process. Polymorphisms are used in mapping the human genome and to elucidate the genetic component of diseases. The following references give further background details on pharmacogenetics and other uses of polymorphism detection: Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

Pharmacogenetics is generally regarded as the study of genetic variation that gives rise to differing response to drugs, while pharmacogenomics is the broader application of genomic technologies to new drug discovery and further characterization of older drugs. Pharmacogenetics considers one or at most a few genes of interest, while pharmacogenomics considers the entire genome. Much of current clinical interest is at the level of pharmacogenetics, involving variation in genes involved in drug metabolism with a particular emphasis on improving drug safety.

Pharmacogenomics is the science of utilising human genetic variation to optimise patient treatment and drug design and discovery. An individual's genetic make up affects each stage of drug response: absorption, metabolism, transport to the target molecule, structure of the intended and/or unintended target molecules, degradation and excretion.

Pharmacogenomics provides the basis for a new generation of personalized pharmaceuticals, the targeting of drug therapies to genetic subpopulations. Currently drugs are developed to benefit the widest possible populations. However the variations in drug reactions attributed to genetic variation are increasingly being taken into account when developing new drugs. There are multiple benefits to such an approach to drug design. The development of genetic tests may reduce the need for the standard trial and error method of drug prescription. Targeted prescriptions would further reduce the incidence of adverse drug reactions, which are estimated to be the fifth ranking cause of death in the United States. Furthermore, dosage decisions can be made on a more informed basis than currently used parameters such as age, sex and weight. Drug discovery and approval processes will likely be speeded up by the specific genetic targeting of candidate drugs. Moreover, this may allow the revival of previously failed candidate drugs. Overall it is expected that the development of personalized pharmaceuticals will reduce the costs of healthcare.

The present invention provides methods for analyzing Sirt1 gene polymorphisms of a subject in a variety of settings that may be before, during or after a medical event including, but not limited to, treatment with an approved drug, treatment with an experimental drug during a clinical trial, trauma, surgery, preventative therapy, vaccination, drug dosing determination, drug efficacy determination, progress or course of therapy with a drug, monitoring disease stage or status or progression, aging, drug addiction, weight loss or gain, cardiovascular or other cardiac-related events, reactions to treatment with a drug, exposure to radiation or other environmental events, exposure to weightlessness or other environmental conditions, exposure to chemical or biological agents (both natural and man-made), and/or diet (ingestion of foodstuffs). In addition, the present invention provides a database of Sirt1 gene polymorphism data for a subject or group of subjects obtained before, during or after a medical event. In one embodiment, the Sirt1 gene polymorphism data obtained according to the present invention is from a subject involved in a clinical trial. In another embodiment, the Sirt1 gene polymorphism data identifies any gene, or collection of genes, that undergoes a change in its level of expression without regard for the function of the encoded protein or association of the gene with any particular function, pathway, disease or other attribute other than its ability to be detected.

In another embodiment, other gene or genes of interest may be known to have an association with the gene expression profile of the subject or the medical event of interest. In one embodiment, for example, another gene known to predispose a subject to a particular disease when expressed, may be monitored before any symptoms are present in the subject to establish a baseline expression level in that subject. Monitoring the Sirt1 gene polymorphisms in the patient may be used to treat, suppress or prevent diseases or disorders related to aging or stress, diabetes, obesity, neurodegenerative diseases, diseases or disorders associated with mitochondrial dysfunction, chemotherapeutic induced neuropathy, neuropathy associated with an ischemic event, ocular diseases and/or disorders, cardiovascular disease, blood clotting disorders, inflammation, and/or flushing, etc. and other chronic and non-chronic diseases as detailed in The Merck Manual of Diagnosis and Therapy (Beers & Berkow, Eds.).

Adverse drug reactions are a principal cause of the low success rate of drug development programs (less than one in four compounds that enter human clinical testing is ultimately approved for use by the U.S. Food and Drug Administration (FDA)). Drug-induced disease or toxicity presents a unique series of challenges to drug developers, as these reactions are often not predictable from preclinical studies and may not be detected in early clinical trials involving small numbers of subjects. When such effects are detected in later stages of clinical development they often result in termination of a drug development program. When a drug is approved despite some toxicity, its clinical use is frequently severely constrained by the possible occurrence of adverse reactions in even a small group of patients. The likelihood of such a compound becoming a first line therapy is small (unless there are no competing products). Clinical trials that use this invention may allow for improved predictions of possible toxic reactions in studies involving a small number of subjects. The methods of this invention offer a quickly derived prediction of likely future toxic effects of an intervention.

Absorption is the first pharmacokinetic parameter to consider when determining variation in drug response. The actual effects of absorption on an individual or group of individuals may be quickly determined using this invention.

Once a drug or candidate therapeutic intervention is absorbed, injected or otherwise enters the bloodstream it is distributed to various biological compartments via the blood. The drug may exist free in the blood, or, more commonly, may be bound with varying degrees of affinity to plasma proteins. One classic source of variation in drug response is attributable to amino acid polymorphisms in serum albumin, which affect the binding affinity of drugs such as warfarin. Consequent variation in levels of free warfarin has a significant effect on the degree of anticoagulation. From the blood a compound diffuses into and is retained in interstitial and cellular fluids of different organs to different degrees. The invention allows for use of genetic haplotyping to be used instead of measurements of the proteins reducing the time and complexity of measurements.

Once absorbed by the gastrointestinal tract, compounds encounter detoxifying and metabolizing enzymes in the tissues of the gastrointestinal system. Many of these enzymes are known to be polymorphic in man and account for well studied variation in pharmacokinetic parameters of many drugs. Subsequently compounds enter the hepatic portal circulation in a process commonly known as first pass. The compounds then encounter a vast array of xenobiotic detoxifying mechanisms in the liver, including enzymes that are expressed solely or at high levels only in liver. These enzymes include the cytochrome P450s, glucuronlytransferases, sulfotransferases, acetyltransferases, methyltransferases, the glutathione conjugating system, flavine monooxygenases, and other enzymes known in the art.

Biotransformation reactions in the liver often have the effect of converting lipophilic compounds into hydrophilic molecules that are then more readily excreted. Variation in these conjugation reactions may affect half-life and other pharmacokinetic parameters. It is important to note that metabolic transformation of a compound not infrequently gives rise to a second or additional compounds that have biological activity greater than, less than, or different from that of the parent compound. Metabolic transformation may also be responsible for producing toxic metabolites.

Genomic expressions can be a precursor to medical events such as clinical responses. The methods of the present invention allow for a prediction of clinical responses on an individual or generally across a population due to an event or intervention. A “Medical Event” is any occurrence that may result in death, may be life-threatening, may require hospitalization, or prolongation of existing hospitalization, may result in persistent or significant disability/incapacity, may be a congenital anomaly/birth defect, may require surgical or non-surgical intervention to prevent one or more of the outcomes listed in this definition, may result in a change in clinical symptoms, or otherwise may result in change in the health of an individual or group of individuals whether naturally or as a result of human intervention.

Different events or interventions may present different responses in gene expression within a subject or between subjects. The invention allows the gene expression responses from differing interventions to be compared to help determine relative effectiveness and toxicity among different interventions and medical events and interventions, including those described in Behrman: Nelson Textbook of Pediatrics, Braunwald: Heart Disease: A Textbook of Cardiovascular Medicine, Brenner: Brenner & Rector's The Kidney, Canale: Campbell's Operative Orthopaedics, Cotran: Robbins Pathologic Basis of Disease, Cummings et al: Otolaryngology—Head and Neck Surgery, DeLee: DeLee and Drez's Orthopaedic Sports Medicine, Duthie: Practice of Geriatric, Feldman: Sleisenger & Fordtran's Gastrointestinal and Liver Disease, Ferri: Ferri's Clinical Advisor, Ferri: Practical Guide to the Care of the Medical Patient, Ford: Clinical Toxicology, Gabbe: Obstetrics: Normal and Problem Pregnancies, Goetz: Textbook of Clinical Neurology, Goldberger: Clinical Electrocardiography, Goldman: Cecil Textbook of Medicine, Grainger: Grainger & Allison's Diagnostic Radiology, Habif: Clinical Dermatology: Color Guide to Diagnosis and Therapy, Hoffman: Hematology: Basic Principles and Practice, Jacobson: Psychiatric Secrets, Johns Hopkins: The Harriet Lane Handbook, Larsen: Williams Textbook of Endocrinology, Long: Principles and Practices of Pediatric Infectious Disease, Mandell: Principles and Practice of Infectious Diseases, Marx: Rosen's Emergency Medicine: Concepts and Clinical Practice, Middleton: Allergy: Principles and Practice, Miller: Anesthesia, Murray & Nadel: Textbook of Respiratory Medicine, Noble: Textbook of Primary Care Medicine, Park: Pediatric Cardiology for Practitioners, Pizzorno: Textbook of Natural Medicine, Rakel: Conn's Current Therapy, Rakel: Textbook of Family Medicine, Ravel: Clinical Laboratory Medicine, Roberts: Clinical Procedures in Emergency Medicine, Ruddy: Kelley's Textbook of Rheumatology, Ryan: Kistner's Gynecology and Women's Health Townsend: Sabiston Textbook of Surgery, Yanoff: Ophthalmology, and Walsh: Campbell's Urology.

The terms “disease” or “condition” are commonly recognized in the art and designate the presence of signs and/or symptoms in an individual or patient that are generally recognized as abnormal. Diseases or conditions may be diagnosed and categorized based on pathological changes. Signs may include any objective evidence of a disease such as changes that are evident by physical examination of a patient or the results of diagnostic tests. Symptoms are subjective evidence of disease or a patient's condition, i.e. the patient's perception of an abnormal condition that differs from normal function, sensation, or appearance, which may include, without limitations, physical disabilities, morbidity, pain, and other changes from the normal condition experienced by an individual. Various diseases or conditions include, but are not limited to; those categorized in standard textbooks of medicine including, without limitation, textbooks of nutrition, allopathic, homeopathic, and osteopathic medicine. In certain aspects of this invention, the disease or condition is selected from the group consisting of the types of diseases listed in standard texts such as Harrison's Principles of Internal Medicine, 14.sup.th Edition (Fauci et al, Eds., McGraw Hill, 1997), or Robbins Pathologic Basis of Disease, 6.sup.th Edition (Cotran et al, Ed. W B Saunders Co., 1998), or the Diagnostic and Statistical Manual of Mental Disorders: DSM-IV, 4.sup.th Edition, (American Psychiatric Press, 1994), or other texts described below.

The term “suffering from a disease or condition” means that a person is either presently subject to the signs and symptoms, or is more likely to develop such signs and symptoms than a normal person in the population. Thus, for example, a person suffering from a condition can include a developing fetus, a person subject to a treatment or environmental condition which enhances the likelihood of developing the signs or symptoms of a condition, or a person who is being given or will be given a treatment which increase the likelihood of the person developing a particular condition. For example, tardive dyskinesia is associated with long-term use of anti-psychotics; dyskinesias, paranoid ideation, psychotic episodes and depression have been associated with use of L-dopa in Parkinson's disease; and dizziness, diplopia, ataxia, sedation, impaired mentation, weight gain, and other undesired effects have been described for various anticonvulsant therapies, alopecia and bone marrow suppression are associated with cancer chemotherapeutic regimens, and immunosuppression is associated with agents to limit graft rejection following transplantation. Thus, methods of the present invention which relate to treatments of patients (e.g., methods for selecting a treatment, selecting a patient for a treatment, and methods of treating a disease or condition in a patient) can include primary treatments directed to a presently active disease or condition, secondary treatments which are intended to cause a biological effect relevant to a primary treatment, and prophylactic treatments intended to delay, reduce, or prevent the development of a disease or condition, as well as treatments intended to cause the development of a condition different from that which would have been likely to develop in the absence of the treatment.

The term “intervention” refers to a process that is intended to produce a beneficial change in the condition of a mammal, e.g., a human, often referred to as a patient. A beneficial change can, for example, include one or more of: restoration of function, reduction of symptoms, limitation or retardation of progression of a disease, disorder, or condition or prevention, limitation or retardation of deterioration of a patient's condition, disease or disorder. Such intervention can involve, for example, nutritional modifications, administration of radiation, administration of a drug, surgery, behavioral modifications, and combinations of these, among others.

The term “intervention” includes administration of “drugs” and “candidate therapeutic agents”. A drug is a chemical entity or biological product, or combination of chemical entities or biological products, administered to a person to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, lipoproteins, and modifications and combinations thereof. A biological product is preferably a monoclonal or polyclonal antibody or fragment thereof such as a variable chain fragment; cells; or an agent or product arising from recombinant technology, such as, without limitation, a recombinant protein, recombinant vaccine, or DNA construct developed for therapeutic, e.g., human therapeutic, use. The term may include, without limitation, compounds that are approved for sale as pharmaceutical products by government regulatory agencies (e.g., U.S. Food and Drug Administration (USFDA or FDA), European Medicines Evaluation Agency (EMEA), and a world regulatory body governing the International Conference of Harmonization (ICH) rules and guidelines), compounds that do not require approval by government regulatory agencies, food additives or supplements including compounds commonly characterized as vitamins, natural products, and completely or incompletely characterized mixtures of chemical entities including natural compounds or purified or partially purified natural products. The term “drug” as used herein is synonymous with the terms “medicine”, “pharmaceutical product”, or “product”. Most preferably the drug is approved by a government agency for treatment of a specific disease or condition. The term “candidate therapeutic agent” refers to a drug or compound that is under investigation, either in laboratory or human clinical testing for a specific disease, disorder, or condition.

The intervention may involve either positive selection or negative selection or both, meaning that the selection can involve a choice that a particular intervention would be an appropriate method to use and/or a choice that a particular intervention would be an inappropriate method to use. Thus, in certain embodiments, the presence of the at least one Sirt1 haplotype may be indicative that the treatment will be effective or otherwise beneficial (or more likely to be beneficial) in the patient. Stating that the treatment will be effective means that the probability of beneficial therapeutic effect is greater than in a person not having the appropriate presence or absence of a particular Sirt1 haplotype. In other embodiments, the presence of the at least one Sirt1 haplotype is indicative that the treatment will be ineffective or contra-indicated for the patient. For example, a treatment may be contra-indicated if the treatment results, or is more likely to result, in undesirable side effects, or an excessive level of undesirable side effects. A determination of what constitutes excessive side-effects will vary, for example, depending on the disease or condition being treated, the availability of alternatives, the expected or experienced efficacy of the treatment, and the tolerance of the patient. As for an effective treatment, this means that it is more likely that desired effect will result from the treatment administration in a patient showing a Sirt1 haplotype consistent with the desired clinical outcome. Also in preferred embodiments, the presence of the at least Sirt1 haplotype is indicative that the treatment is both effective and unlikely to result in undesirable effects or outcomes, or vice versa (is likely to have undesirable side effects but unlikely to produce desired therapeutic effects).

The invention may be useful in predicting a patient's tolerance to an intervention. In reference to response to a treatment, the term “tolerance” refers to the ability of a patient to accept a treatment, based, e.g., on deleterious effects and/or effects on lifestyle. Frequently, the term principally concerns the patients' perceived magnitude of deleterious effects such as nausea, weakness, dizziness, and diarrhea, among others. Such experienced effects can, for example, be due to general or cell-specific toxicity, activity on non-target cells, cross-reactivity on non-target cellular constituents (non-mechanism based), and/or side effects of activity on the target cellular substituents (mechanism based), or the cause of toxicity may not be understood. In any of these circumstances one may identify an association between the undesirable effects and Sirt1 haplotype.

Adverse responses to drugs constitute a major medical problem, as shown in two recent meta-analyses (Lazarou et al, “Incidence of Adverse Drug Reactions in Hospitalized Patients: A Meta-Analysis of Prospective Studies”, 279 JAMA 1200-1205 (1998); and Bonn, “Adverse Drug Reactions Remain a Major Cause of Death”, 351 LANCET 1183 (1998). An estimated 2.2 million hospitalized patients in the United Stated had serious-adverse drug reactions in 1994, with an estimated 106,000 deaths (Lazarou et al.). To the extent that some of these adverse events are predictable based on changes in RNA expression, the identification of changes that are predictive of such effects will allow for more effective and safer drug use.

The present invention also has uses in the area of eliminating treatments. The phrase “eliminating a treatment” refers to removing a possible treatment from consideration, e.g., for use with a particular patient based on one or more changes in Sirt1 haplotype, or to stopping the administration of a treatment which was in the course of administration.

Also in preferred embodiments, the method of selecting a treatment involves selecting a method of administration of a compound, combination of compounds, or pharmaceutical composition, for example, selecting a suitable dosage level and/or frequency of administration, and/or mode of administration of a compound. The method of administration can be selected to provide better, preferably maximum therapeutic benefit. In this context, “maximum” refers to an approximate local maximum based on the parameters being considered, not an absolute maximum. The term “suitable dosage level” refers to a dosage level which provides a therapeutically reasonable balance between pharmacological effectiveness and deleterious effects. Often this dosage level is related to the peak or average serum levels resulting from administration of a drug at the particular dosage level. Similarly, a “frequency of administration” refers to how often in a specified time period a treatment is administered, e.g., once, twice, or three times per day, every other day, once per week, etc. For a drug or drugs, the frequency of administration is generally selected to achieve a pharmacologically effective average or peak serum level without excessive deleterious effects (and preferably while still being able to have reasonable patient compliance for self-administered drugs). Thus, it is desirable to maintain the serum level of the drug within a therapeutic window of concentrations for the greatest percentage of time possible without such deleterious effects as would cause a prudent physician to reduce the frequency of administration for a particular dosage level.

Thus, in connection with the administration of a drug, a drug which is “effective against” a disease or condition indicates that administration in a clinically appropriate manner results in a beneficial effect for at least a statistically significant fraction of patients, such as a improvement of symptoms, a cure, a reduction in disease load, reduction in tumor mass or cell numbers, extension of life, improvement in quality of life, or other effect generally recognized as positive by medical doctors familiar with treating the particular type of disease or condition.

Effectiveness is measured in a particular population. In conventional drug development the population is generally every subject who meets the enrollment criteria (i.e. has the particular form of the disease or condition being treated). It is an aspect of the present invention that segmentation of a study population by genetic criteria can provide the basis for identifying a subpopulation in which a drug is effective against the disease or condition being treated.

The term “deleterious effects” refers to physical effects in a patient caused by administration of a treatment which are regarded as medically undesirable. Thus, for example, deleterious effects can include a wide spectrum of toxic effects injurious to health such as death of normally functioning cells when only death of diseased cells is desired, nausea, fever, inability to retain food, dehydration, damage to critical organs such as arrhythmias, renal tubular necrosis, fatty liver, or pulmonary fibrosis leading to coronary, renal, hepatic, or pulmonary insufficiency among many others. In this regard, the term “adverse reactions” refers to those manifestations of clinical symptomology of pathological disorder or dysfunction induced by administration of a drug, agent, or candidate therapeutic intervention. In this regard, the term “contraindicated” means that a treatment results in deleterious effects such that a prudent medical doctor treating such a patient would regard the treatment as unsuitable for administration. Major factors in such a determination can include, for example, availability and relative advantages of alternative treatments, consequences of non-treatment, and permanency of deleterious effects of the treatment.

It is recognized that many treatment methods, e.g., administration of certain compounds or combinations of compounds, may produce side-effects or other deleterious effects in patients. Such effects can limit or even preclude use of the treatment method in particular patients, or may even result in irreversible injury, disorder, dysfunction, or death of the patient. Thus, in certain embodiments, the variance information is used to select both a first method of treatment and a second method of treatment. Usually the first treatment is a primary treatment which provides a physiological effect directed against the disease or condition or its symptoms. The second method is directed to reducing or eliminating one or more deleterious effects of the first treatment, e.g., to reduce a general toxicity or to reduce a side effect of the primary treatment. Thus, for example, the second method can be used to allow use of a greater dose or duration of the first treatment, or to allow use of the first treatment in patients for whom the first treatment would not be tolerated or would be contra-indicated in the absence of a second method to reduce deleterious effects or to potentiate the effectiveness of the first treatment.

In a related aspect, the invention provides a method for selecting a method of treatment for a patient suffering from a disease or condition by comparing changes in gene expression to pharmacokinetic parameters, or organ and tissue damage, or inordinate immune response, which are indicative of the effectiveness or safety of at least one method of treatment.

Similar to the above aspect, in preferred embodiments, at least one method of treatment involves the administration of a compound effective in at least some patients with a disease or condition; the presence or absence of the at least one change in gene expression is indicative that the treatment will be effective in the patient; and/or the presence or absence of the at least one change in gene expression is indicative that the treatment will be ineffective or contra-indicated in the patient; and/or the treatment is a first treatment and the presence or absence of the at least one change in gene expression is indicative that a second treatment will be beneficial to reduce a deleterious effect or potentiate the effectiveness of the first treatment; and/or the at least one treatment is a plurality of methods of treatment. For a plurality of treatments, preferably the selecting involves determining whether any of the methods of treatment will be more effective than at least one other of the plurality of methods of treatment. Yet other embodiments are provided as described for the preceding aspect in connection with methods of treatment using administration of a compound; treatment of various diseases, and variances in genetic expressions.

In addition to the basic method of treatment, often the mode of administration of a given compound as a treatment for a disease or condition in a patient is significant in determining the course and/or outcome of the treatment for the patient. Thus, the invention also provides a method for selecting a method of administration of a compound to a patient suffering from a disease or condition, by determining changes in gene expression where such presence or absence is indicative of an appropriate method of administration of the compound. Preferably, the selection of a method of treatment (a treatment regimen) involves selecting a dosage level or frequency of administration or route of administration of the compound or combinations of those parameters. In preferred embodiments, two or more compounds are to be administered, and the selecting involves selecting a method of administration for one, two, or more than two of the compounds, jointly, concurrently, or separately. As understood by those skilled in the art, such plurality of compounds may be used in combination therapy, and thus may be formulated in a single drug, or may be separate drugs administered concurrently, serially, or separately. Other embodiments are as indicated above for selection of second treatment methods, methods of identifying Sirt1 haplotypes, and methods of treatment as described for aspects above.

In another aspect, the invention provides a method for selecting a patient for administration of a method of treatment for a disease or condition, or of selecting a patient for a method of administration of a treatment, by analyzing Sirt1 haplotype as identified above in peripheral blood of a patient, where the Sirt1 haplotype is indicative that the treatment or method of administration that will be effective in the patient.

In one embodiment, the disease or the method of treatment is as described in aspects above, specifically including, for example, those described for selecting a method of treatment.

In another aspect, the invention provides a method for identifying patients with enhanced or diminished response or tolerance to a treatment method or a method of administration of a treatment where the treatment is for a disease or condition in the patient. The method involves correlating one or more Sirt1 haplotypes as identified in aspects above in a plurality of patients with response to a treatment or a method of administration of a treatment. The correlation may be performed by determining the one or more Sirt1 haplotypes in the plurality of patients and correlating the presence or absence of each of the changes (alone or in various combinations) with the patient's response to treatment. The Sirt1 haplotype(s) may be previously known to exist or may also be determined in the present method or combinations of prior information and newly determined information may be used. The enhanced or diminished response should be statistically significant, preferably such that p=0. 10 or less, more preferably 0.05 or less, and most preferably 0.02 or less. A positive correlation between the presence of one or more Sirt1 haplotypes and an enhanced response to treatment is indicative that the treatment is particularly effective in the group of patients showing certain patterns of Sirt1 haplotypes. A positive correlation of the presence of the one or more expression changes with a diminished response to the treatment is indicative that the treatment will be less effective in the group of patients having those variances. Such information is useful, for example, for selecting or de-selecting patients for a particular treatment or method of administration of a treatment, or for demonstrating that a group of patients exists for which the treatment or method of treatment would be particularly beneficial or contra-indicated. Such demonstration can be beneficial, for example, for obtaining government regulatory approval for a new drug or a new use of a drug.

Preferred embodiments include drugs, treatments, variance identification or determination, determination of effectiveness, and/or diseases as described for aspects above or otherwise described herein.

In other embodiments, the correlation of patient responses to therapy according to Sirt1 haplotype is carried out in a clinical trial, e.g., as described herein according to any of the variations described. Detailed description of methods for associating variances with clinical outcomes using clinical trials is provided below. Further, in preferred embodiments the correlation of pharmacological effect (positive or negative) to Sirt1 haplotype in such a clinical trial is part of a regulatory submission to a government agency leading to approval of the drug. Most preferably the compound or compounds. would not be approvable in the absence of this data.

As indicated above, in aspects of this invention involving selection of a patient for a treatment, selection of a method or mode of administration of a treatment, and selection of a patient for a treatment or a method of treatment, the selection may be positive selection or negative selection. Thus, the methods can include eliminating a treatment for a patient, eliminating a method or mode of administration of a treatment to a patient, or elimination of a patient for a treatment or method of treatment.

The present invention provides a method for treating a patient at risk for drug responsiveness, i.e., efficacy differences associated with pharmacokinetic parameters, and safety concerns, i.e. drug-induced disease, disorder, or dysfunction or diagnosed with organ failure or a disease associated with drug-induced organ failure. The methods include identifying such a patient and determining the patient's changes in genetic expressions. The patient identification can, for example, be based on clinical evaluation using conventional clinical metrics.

In a related aspect, the invention provides a method for identifying a patient for participation in a clinical trial of a therapy for the treatment of a disease, disorder, or dysfunction, or an associated drug-induced toxicity. The method involves determining the changes in genetic expression of a patient with (or at risk for) a disease, disorder, or dysfunction. The trial would then test the hypothesis that a statistically significant difference in response to a treatment can be demonstrated between two groups of patients each defined changes or lack of changes in genetic expression. Said response may be a desired or an undesired response. In a preferred embodiment, the treatment protocol involves a comparison of placebo vs. treatment response rates in two or more groups. For example a group with no changes in expression of one or more genes of interest may be compared to a group with changes in one or more gene expressions.

In another preferred embodiment, patients in a clinical trial can be grouped (at the end of the trial) according to treatment response, and statistical methods can be used to compare changes to gene expression in these groups. For example responders can be compared to nonresponders, or patients suffering adverse events can be compared to those not experiencing such effects. Alternatively response data can be treated as a continuous variable and the ability of gene expression to predict response can be measured. In a preferred embodiment, patients who exhibit extreme responses are compared with all other patients or with a group of patients who exhibit a divergent extreme response. For example if there is a continuous or semi-continuous measure of treatment response (for example the Alzheimer's Disease Assessment Scale, the Mini-Mental State Examination or the Hamilton Depression Rating Scale) then the 10% of patients with the most favorable responses could be compared to the 10% with the least favorable, or the patients one standard deviation above the mean score could be compared to the remainder, or to those one standard deviation below the mean score. One useful way to select the threshold for defining a response is to examine the distribution of responses in a placebo group. If the upper end of the range of placebo responses is used as a lower threshold for an ‘outlier response’ then the outlier response group should be almost free of placebo responders. This is a useful threshold because the inclusion of placebo responders in a ‘true’ response group decreases the ability of statistical methods to detect a changes in gene expression between responders and nonresponders.

In a related aspect, the invention provides a method for developing a disease management protocol that entails diagnosing a patient with a disease or a disease susceptibility, determining the changes in gene expression of the patient at a gene or genes correlated with treatment response and then selecting an optimal treatment based on the disease and the changes in gene expression. The disease management protocol may be useful in an education program for physicians, other caregivers or pharmacists; may constitute part of a drug label; or may be useful in a marketing campaign.

“Disease management protocol” or “treatment protocol” is a means for devising a therapeutic plan for a patient using laboratory, clinical and genetic data, including the patient's diagnosis and genotype. The protocol clarifies therapeutic options and provides information about probable prognoses with different treatments. The treatment protocol may provide an estimate of the likelihood that a patient will respond positively or negatively to a therapeutic intervention. The treatment protocol may also provide guidance regarding optimal drug dose and administration and likely timing of recovery or rehabilitation. A “disease management protocol” or “treatment protocol” may also be formulated for asymptomatic and healthy subjects in order to forecast future disease risks based on laboratory, clinical and gene expression variables. In this setting the protocol specifies optimal preventive or prophylactic interventions, including use of compounds, changes in diet or behavior, or other measures. The treatment protocol may include the use of a computer program.

In other embodiments of above aspects involving prediction of drug efficacy, the prediction of drug efficacy involves candidate therapeutic interventions that are known or have been identified to be affected by pharmacokinetic parameters, i.e. absorption, distribution, metabolism, or excretion. These parameters may be associated with hepatic or extra-hepatic biological mechanisms. Preferably the candidate therapeutic intervention will be effective in patients with the.known changes in genetic expression but have a risk of drug ineffectiveness, i.e. nonresponsive to a drug or candidate therapeutic intervention.

In other embodiments, the above methods are used for or include identification of a safety or toxicity concern involving a drug-induced disease, disorder, or dysfunction and/or the likelihood of occurrence and/or severity of said disease, disorder, or dysfunction.

In other embodiments, the invention is suitable for identifying a patient with non-drug-induced disease, disorder, or dysfunction but with dysfunction related to aberrant enzymatic metabolism or excretion of endogenous biologically relevant molecules or compounds.

### 6. Sirtuin Modulating Compounds

In various embodiments, the methods described herein involve administration of a sirtuin modulating compound. A sirtuin-modulating compound refers to a compound that may either up regulate (e.g., activate or stimulate), down regulate (e.g., inhibit or suppress) or otherwise change a functional property or biological activity of a sirtuin protein. Sirtuin-modulating compounds may act to modulate a sirtuin protein either directly or indirectly. In certain embodiments, a sirtuin-modulating compound may be a sirtuin-activating compound or a sirtuin-inhibiting compound.

A sirtuin-activating compound refers to a compound that increases the level of a sirtuin protein and/or increases at least one activity of a sirtuin protein. In an exemplary embodiment, a sirtuin-activating compound may increase at least one biological activity of a sirtuin protein by at least about 10%, 25%, 50%, 75%, 100%, or more. In exemplary embodiments, sirtuin activating compounds increase deacetylase activity of a sirtuin protein, e.g., increased deacteylation of one or more sirtuin substrates. Exemplary sirtuin activating compounds include flavones, stilbenes, flavanones, isoflavanones, catechins, chalcones, tannins and anthocyanidins. Exemplary stilbenes include hydroxystilbenes, such as trihydroxystilbenes, e.g., 3,5,4′—trihydroxystilbene (“resveratrol”). Resveratrol is also known as 3,4′,5-stilbenetriol. Tetrahydroxystilbenes, e.g., piceatannol, are also encompassed. Hydroxychalones including trihydroxychalones, such as isoliquiritigenin, and tetrahydroxychalones, such as butein, can also be used. Hydroxyflavones including tetrahydroxyflavones, such as fisetin, and pentahydroxyflavones, such as quercetin, can also be used. Other sirtuin activating compounds are described in U.S. Patent Application Publication No. 2005/0096256 and PCT Application Nos. PCT/US06/002092, PCT/US06/007746, PCT/US06/007744, PCT/US06/007745, PCT/US06/007778, PCT/US06/007656, PCT/US06/007655, PCT/US06/007773, PCT/US06/030661, PCT/US06/030512, PCT/US06/030511, PCT/US06/030510, and PCT/US06/030660.

A sirtuin-inhibiting compound refers to a compound that decreases the level of a sirtuin protein and/or decreases at least one activity of a sirtuin protein. In an exemplary embodiment, a sirtuin-inhibiting compound may decrease at least one biological activity of a sirtuin protein by at least about 10%, 25%, 50%, 75%, 100%, or more. In exemplary embodiments, sirtuin inhibiting compounds decrease deacetylase activity of a sirtuin protein, e.g., decreased deacteylation of one or more sirtuin substrates. Exemplary sirtuin inhibitors include, for example, sirtinol and analogs thereof (see e.g., Napper et al., J. Med. Chem. 48: 8045-54 (2005)), nicotinamide (NAD⁺) and suramin and analogs thereof. Other sirtuin inhibiting compounds are described in U.S. Patent Application Publication No. 2005/0096256, PCT Publication No. WO2005/002527, and PCT Application Nos. PCT/US06/007746, PCT/US06/007744, PCT/US06/007745, PCT/US06/007778, PCT/US06/007656, PCT/US06/007655, PCT/US06/007773 and PCT/US06/007742.

Exemplary sirtuin activating compounds are provided below. In one embodiment, the methods disclosed herein utilize administration of a sirtuin-modulating compound of Formula (I):

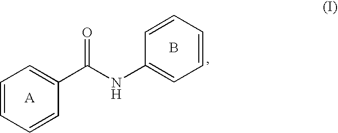

or a salt thereof, where:

Ring A is optionally substituted, fused to another ring or both; and

Ring B is substituted with at least one carboxy, substituted or unsubstituted arylcarboxamine, substituted or unsubstituted aralkylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

In one embodiment, the methods disclosed herein utilize administration of a sirtuin-modulating compound of Formula (CII):

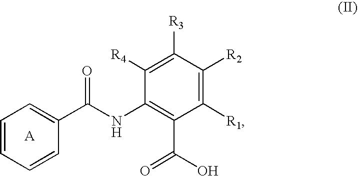

or a salt thereof, where:

Ring A is optionally substituted;

R₁, R₂, R₃ and R₄ are independently selected from the group consisting of —H, halogen, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₅ and R₆ are independently —H, a substituted or unsubstituted alkyl group, a substituted or unsubstituted aryl group or a substituted or unsubstituted heterocyclic group; and

n is 1 or 2.

I In one embodiment, the methods disclosed herein utilize administration of a sirtuin-modulating compound of Formula (I):

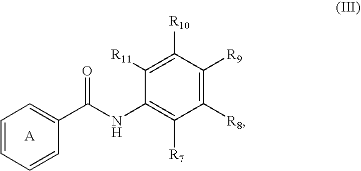

or a salt thereof, where:

Ring A is optionally substituted;

R₅ and R₆ are independently —H, a substituted or unsubstituted alkyl group, a substituted or unsubstituted aryl group or a substituted or unsubstituted heterocyclic group;

R₇, R₉, R₁₀ and R₁₁ are independently selected from the group consisting of —H, halogen, —R₅, —OR₅, —CN, —CO₂R₅, —OCOR₅, —OCO₂R₅, —C(O)NR₅R₆, —OC(O)NR₅R₆, —C(O)R₅, —COR₅, —SR₅, —OSO₃H, —S(O)ₙR₅, —S(O)ₙOR₅, —S(O)ₙNR₅R₆, —NR₅R₆, —NR₅C(O)OR₆, —NR₅C(O)R₆ and —NO₂;

R₈ is a polycyclic aryl group; and

n is 1 or 2.

In one embodiment, the methods disclosed herein utilize administration of a sirtuin-modulating compound of Formula (IV):

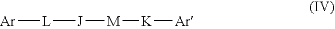

or a salt thereof, wherein:

each Ar and Ar′ is independently an optionally substituted carbocyclic or heterocyclic aryl group;

L is an optionally substituted carbocyclic or heterocyclic arylene group;

each J and K is independently NR₁′, O, S, or is optionally independently absent; or when J is NR₁′, R₁′ is a C1-C4 alkylene or C2-C4 alkenylene attached to Ar′ to form a ring fused to Ar′; or when K is NR₁′, R₁′ is a C1-C4 alkylene or C2-C4 alkenylene attached to L to form a ring fused to L;

each M is C(O), S(O), S(O)₂, or CR₁′R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂′; OC(O)R₂′; S(O)₂R₂′; S(O)₂NR₂′R₂′; NR₂° C.(O)NR₂′R₂′; NR₂° C.(O)C(O)R₂′; NR₂° C.(O)R₂′; NR₂′(COOR₂′); NR₂° C.(O)R₅′; NR₂′S(O)₂NR₂′R₂′; NR₂′S(O)₂R₂′; NR₂′S(O)₂R₅′; NR₂° C.(O)C(O)NR₂′R₂′; NR₂° C.(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₈′, NR₈′R₈′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur; oxygen; CF₃; haloalkyl; SR₂′; OR₂′; OC(O)R₂′; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′, or aryl;

each R₆′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur; oxygen; CF₃; haloalkyl; SR₇′; OR₇′; NR₇′R₇′; NR₇′R₈′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C 10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COORC₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉° C.(O)NR₉′R₉′; NR₉° C.(O)C(O)R₉′; NR₉° C.(O)R₆′; NR₉′S(O)₂NR₉′R₉′; NR₉′S(O)₂R₆′; NR₉° C.(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In one embodiment, the methods disclosed herein utilize administration of a sirtuin-modulating compound of Formula (IVa):

Het-L-Q-Ar′ (IVa)

or a salt thereof, where:

Het is an optionally substituted heterocyclic aryl group;

L is an optionally substituted carbocyclic or heterocyclic arylene group;

Ar′ is an optionally substituted carbocyclic or heterocyclic aryl group; and

Q is selected from —NR₁′—C(O)—, —NR₁′—S(O)₂—, —NR₁′—C(O)—NR₁′—, —NR₁′—C(S)—NR₁′—, —NR₁′—C(O)—CR₁′R′₁NR₁′—, —NR₁′—C(═NR₁′)—NR₁′—, —C(O)—NR₁′—, —C(O)—NR₁′—S(O)₂—, —NR₁′—, —CR₁′R′₁—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—,

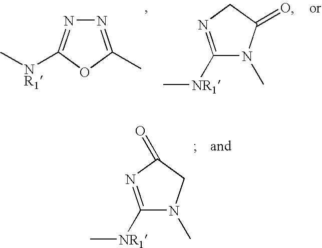

each R₁′ is independently selected from H or optionally substituted C₁-C₃ straight or branched alkyl, wherein:

when Het is a polycyclic heteroaryl, L is an optionally substituted phenylene, Q and Het are attached to L in a meta orientation, and Ar′ is optionally substituted phenyl; then Q is not —NH—C(O)—.

In one embodiment, the methods disclosed herein utilize administration of a sirtuin-modulating compound of Formula (V):

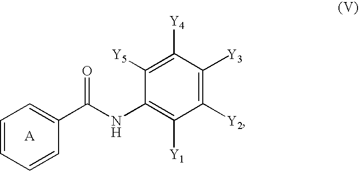

or a salt thereof, wherein:

Ring A is optionally substituted with at least one R₁′ group;

Y₁, Y₂, Y₃, Y₄, and Y₅ are independently R₁′;

each R₁′ is independently selected from H, C1-C10 alkyl; C2-C10 alkenyl; C2-C10 alkynyl; C3-C10 cycloalkyl; C4-C10 cycloalkenyl; aryl; R₅′; halo; haloalkyl; CF₃; SR₂′; OR₂′; NR₂′R₂′; NR₂′R₃′; COOR₂′; NO₂; CN; C(O)R₂′; C(O)C(O)R₂′; C(O)NR₂′R₂′; OC(O)R₂′; S(O)₂R₂′; S(O)₂NR₂′R₂′; NR₂° C.(O)NR₂′R₂′; NR₂° C.(O)C(O)R₂′; NR₂° C.(O)R₂′; NR₂′(COOR₂′); NR₂° C.(O)R₅′; NR₂′S(O)₂NR₂′R₂′; NR₂′S(O)₂R₂′; NR₂′S(O)₂R₅′; NR₂° C.(O)C(O)NR₂′R₂′; NR₂° C.(O)C(O)NR₂′R₃′; C1-C10 alkyl substituted with aryl, R₄′ or R₅′; or C2-C10 alkenyl substituted with aryl, R₄′ or R₅′;

each R₂′ is independently H; C1-C10 alkyl; C2-C10 alkenyl; C2-C10 alkynyl; C3-C10 cycloalkyl; C4-C10 cycloalkenyl; aryl; R₆′; C1-C10 alkyl substituted with 1-3 independent aryl, R₄′ or R₆′ groups; C3-C10 cycloalkyl substituted with 1-3 independent aryl, R₄′ or R₆′ groups; or C2-C10 alkenyl substituted with 1-3 independent aryl, R₄′ or R₆′;

each R₃′ is independently C(O)R₂′, COOR₂′, or S(O)₂R₂′;

each R₄′ is independently halo, CF₃, SR₇′, OR₇′, OC(O)R₇′, NR₇′R₇′, NR₇′R₈′, NR₈′R₈′, COOR₇′, NO₂, CN, C(O)R₇′, or C(O)NR₇′R₇′;

each R₅′ is independently a 5-8 membered monocyclic, 8-12 membered bicyclic, or 11-14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; aryl; R₆′; halo; sulfur; oxygen; CF₃; haloalkyl; SR₂′; OR₂′; OC(O)R₂′; NR₂′R₂′; NR₂′R₃′; NR₃′R₃′; COOR₂′; NO₂; CN; C(O)R₂′; C(O)NR₂′R₂′; C1-C10 alkyl substituted with 1-3 independent R₄′, R₆′, or aryl; or C2-C10 alkenyl substituted with 1-3 independent R₄′, R₆′, or aryl;

each R₆′ is independently a 5-8 membered monocyclic, 8-12 membered bicyclic, or 11 - 14 membered tricyclic ring system comprising 1-3 heteroatoms if monocyclic, 1-6 heteroatoms if bicyclic, or 1-9 heteroatoms if tricyclic, said heteroatoms selected from O, N, or S, which may be saturated or unsaturated, and wherein 0, 1, 2 or 3 atoms of each ring may be substituted by a substituent independently selected from C1-C10 alkyl; C2-C10 alkenyl; C2-C10 alkynyl; C3-C10 cycloalkyl; C4-C10 cycloalkenyl; halo; sulfur; oxygen; CF₃; haloalkyl; SR₇′; OR₇′; NR₇′R₇′; NR₇′R₈′; NR₈′R₈′; COOR₇′; NO₂; CN; C(O)R₇′; or C(O)NR₇′R₇′;

each R₇′ is independently H, C1-C10 alkyl; C2-C10 alkenyl; C2-C10 alkynyl; C3-C10 cycloalkyl; C4-C10 cycloalkenyl; haloalkyl; C1-C10 alkyl optionally substituted with 1-3 independent C1-C 10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′; or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀R₁₀′, COOR₁₀′, NO₂, CN, C(O)R₁₀′, C(O)NR₁₀′R₁₀′, NHC(O)R₁₀′, or OC(O)R₁₀′;

each R₈′ is independently C(O)R₇′, COOR₇′, or S(O)₂R₇′;

each R₉′ is independently H, C1-C10 alkyl, C2-C10 alkenyl, C2-C10 alkynyl, C3-C10 cycloalkyl, C4-C10 cycloalkenyl, or phenyl optionally substituted with 1-3 independent C1-C10 alkyl, C2-C10 alkenyl, C2-C10 alkynyl, C3-C10 cycloalkyl, C4-C10 cycloalkenyl, halo, CF₃, OR₁₀′, SR₁₀′, NR₁₀′R₁₀′, COOR₁₀′, NO₂, CN, C(O)R₁₀′, C(O)NR₁₀′R₁₀′, NHC(O)R₁₀′, or OC(O)R₁₀′;

each R₁₀′ is independently H; C1-C10 alkyl; C2-C10 alkenyl; C2-C10 alkynyl; C3-C10 cycloalkyl; C4-C10 cycloalkenyl; C1-C10 alkyl optionally substituted with halo, CF₃, OR₁₁′, SR₁₁′, NR₁₁′R₁₁′, COOR₁₁′, NO₂, CN; or phenyl optionally substituted with halo, CF₃, OR₁₁′, SR₁₁′, NR₁₁′R₁₁′, COOR₁₁′, NO₂, CN;

each R₁₁′ is independently H; C1-C10 alkyl; C3-C10 cycloalkyl or phenyl;

each haloalkyl is independently a C1-C10 alkyl substituted with one or more halogen atoms, selected from F, Cl, Br, or I, wherein the number of halogen atoms may not exceed that number that results in a perhaloalkyl group; and

each aryl is independently a 5- to 7-membered monocyclic ring system or a 9- to 12-membered bicyclic ring system optionally substituted with 1-3 independent C1-C10 alkyl; C2-C10 alkenyl; C2-C10 alkynyl; C3-C10 cycloalkyl; C4-C10 cycloalkenyl; R₆′; halo; haloalkyl; CF₃; OR₉′; SR₉′; NR₉′R₉′; COOR₉′; NO₂; CN; C(O)R₉′; C(O)C(O)R₉′; C(O)NR₉′R₉′; S(O)₂R₉′; N(R₉′)C(O)R₉′; N(R₉′)(COOR₉′); N(R₉′)S(O)₂R₉′; S(O)₂NR₉′R₉′; OC(O)R₉′; NR₉° C.(O)NR₉′R₉′; NR₉° C.(O)C(O)R₉′; NR₉° C.(O)R₆′; NR₉′S(O)₂NR₉′R₉′; NR₉′S(O)₂R₆′; NR₉° C.(O)C(O)NR₉′R₉′; C1-C10 alkyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; C2-C10 alkenyl substituted with 1-3 independent R₆′, halo, CF₃, OR₉′, SR₉′, NR₉′R₉′, COOR₉′, NO₂, CN, C(O)R₉′, C(O)NR₉′R₉′, NHC(O)R₉′, NH(COOR₉′), S(O)₂NR₉′R₉′, OC(O)R₉′; or R₉′.

In one embodiment, the methods disclosed herein utilize administration of a sirtuin-modulating compound of Formula (VI):

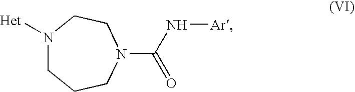

or a salt thereof, wherein:

Het is an optionally substituted heterocyclic aryl group; and

Ar′ is an optionally substituted carbocyclic or heterocyclic aryl group.

The invention also includes prodrugs and metabolites of the compounds disclosed herein.

In one embodiment, the methods disclosed herein utilize administration of a sirtuin-modulating compound of Formula (VII):

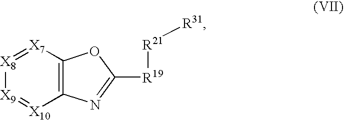

or a salt thereof, wherein:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

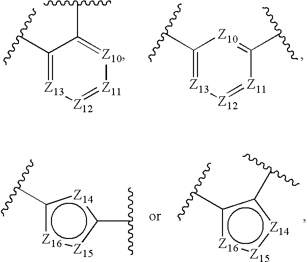

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁° C.(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

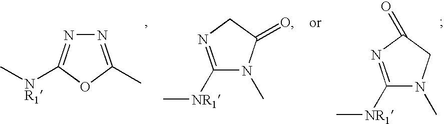

and

R³¹ is selected from an optionally substituted monocyclic or bicyclic aryl, or an optionally substituted monocyclic or bicyclic heteroaryl, with the provisos that said compound is not:

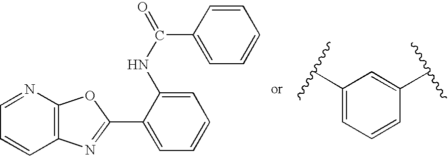

that when R¹⁹ is and R²¹ is —NHC(O)—, R³¹ is not an optionally substituted phenyl.

In certain embodiments, compounds of Structural Formula (VII) have the following values:

each of X₇, X₈, X₉ and X₁₀ is independently selected from N, CR²⁰, or CR₁′, wherein:

each R²⁰ is independently selected from H or a solubilizing group;

each R₁′ is independently selected from H or optionally substituted C₁-C₃ straight or branched alkyl;

one of X₇, X₈, X₉ and X₁₀ is N and the others are selected from CR²⁰ or CR₁′; and

zero to one R²⁰ is a solubilizing group;

R¹⁹ is selected from:

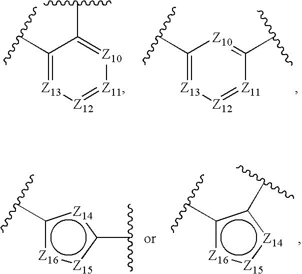

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

said compound is not:

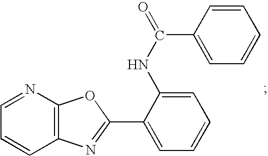

and

when X₈ and X₉ are each independently selected from CR²⁰ or CR₁′, R¹⁹ is

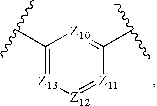

and each of Z₁₀, Z₁₁, Z₁₂ and Z₁₃ is independently selected from CR²⁰, or CR₁′, then:
a) at least one of X₈ and X₉ is not CH; or
b) at least one of Z₁₀, Z₁₁, Z₁₂ and Z₁₃ is CR²⁰, wherein R²⁰ is a solubilizing group.

In one embodiment, the methods disclosed herein utilize administration of a sirtuin-modulating compound of Formula (VIII):

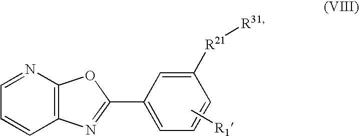

or a salt thereof, wherein:

R₁′ is selected from H or optionally substituted C₁-C₃ straight or branched alkyl;

R²¹ is selected from —NR₁′—C(O)—, —NR₁′—S(O)₂—, —NR₁′—C(O)—NR₁′, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

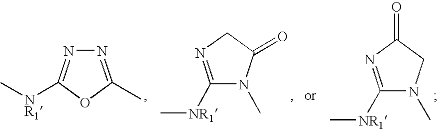

and

R³¹ is selected from an optionally substituted monocyclic or bicyclic aryl, or an optionally substituted monocyclic or bicyclic heteroaryl, with the provisos that:

when R₁′ is methyl, and R²¹ is —NH—C(O)—, R³¹ is not

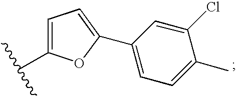

1-methoxynaphthyl; 2-methoxynaphthyl; or unsubstituted 2-thienyl;

when R₁′ is methyl, and R²¹ is —NH—C(O)—CH═CH—, R³¹ is not

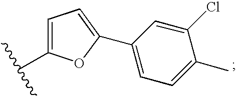

when R₁′ is methyl, and R²¹ is —NH—C(O)—CH—O—, R³¹ is not unsubstituted naphthyl; 2-methoxy, 4-nitrophenyl; 4-chloro, 2-methylphenyl; or 4-t-butylphenyl; and

when R²¹ is —NH—C(O)—, R³¹ is not optionally substituted phenyl.

In one embodiment, the methods disclosed herein utilize administration of a sirtuin-modulating compound of Formula (IX):

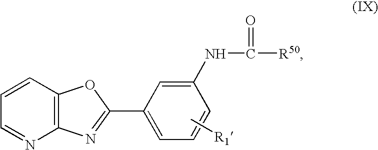

or a salt thereof, wherein;

R₁′ is selected from H or optionally substituted C₁-C₃ straight or branched alkyl; and

R⁵⁰ is selected from 2,3-dimethoxyphenyl, phenoxyphenyl, 2-methyl-3-methoxyphenyl, 2-methoxy-4-methylphenyl, or phenyl substituted with 1 to 3 substituents, wherein one of said substituents is a solubilizing group; with the provisos that R⁵⁰ is not substituted simultaneously with a solubilizing group and a nitro group, and R⁵⁰ is not singly substituted at the 4-position with cyclic solubilizing group or at the 2-position with a morpholino group.

In one embodiment, the methods disclosed herein utilize administration of a sirtuin-modulating compound of Formula (X):

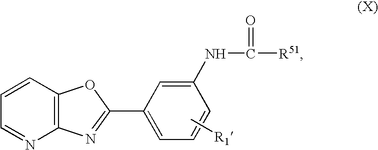

or a salt thereof, wherein:

R₁′ is selected from H or optionally substituted C₁-C₃ straight or branched alkyl; and

R⁵¹ is selected from an ooptionally substituted monocyclic heteroaryl, an optionally substituted bicyclic heteroaryl, or an optionally substituted naphthyl, wherein R⁵¹ is not chloro-benzo(b)thienyl, unsubstituted benzodioxolyl, unsubstituted benzofuranyl, methyl- benzofuranyl, unsubstituted furanyl, phenyl-, bromo-, or nitro-furyl, chlorophenyl- isoxazolyl, oxobenzopyranyl, unsubstituted naphthyl, methoxy-, methyl-, or halo- naphthyl, unsubstituted thienyl, unsubstituted pyridinyl, or chloropyridinyl.

In one embodiment, the methods disclosed herein utilize administration of a sirtuin-modulating compound of Formula (XI):

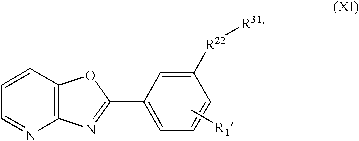

or a salt thereof, wherein:

R₁′ is selected from H or optionally substituted C₁-C₃ straight or branched alkyl;

R²² is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R′₁—CR₁′R′₁—, —NR₁′—C(S)—NR₁′—CR₁′R′₁—CR₁′R′₁—, —NR₁′—C(O)—O— or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl, with the provisos that:

when R²² is —NH—C(O)—CH═CH—, R³¹ is not unsubstituted furyl, 5-(2-methyl-3-chlorophenyl)-furanyl, 2,4-dichlorophenyl, 3,5-dichloro-2-methoxyphenyl, 3-nitrophenyl, 4-chlorophenyl, 4-chloro-3-nitrophenyl, 4-isopropylphenyl, 4-methoxyphenyl, 2-methoxy-5-bromophenyl, or unsubstituted phenyl;

when R²² is —NH—C(O)—CH₂—, R³¹ is not 3,4-dimethoxyphenyl, 4-chlorophenyl, or unsubstituted phenyl;

when R²² is —NH—C(O)—CH₂—O—, R³¹ is not 2,4-dimethyl-6-nitrophenyl, 2- or 4-nitrophenyl, 4-cyclohexylphenyl, 4-methoxyphenyl, unsubstituted naphthyl, or unsubstituted phenyl, or phenyl monosubstituted, disubstituted or trisubstituted solely with substituents selected from straight- or branched-chain alkyl or halo;

when R²² is —NH—C(O)—CH(CH₃)—O—, R³¹ is not 2,4-dichlorophenyl, 4-chlorophenyl, or unsubstituted phenyl; and

when R²² is —NH—S(O)₂—, R³¹ is not unsubstituted phenyl.

In one embodiment, the methods disclosed herein utilize administration of a sirtuin-modulating compound of Formula (XII):

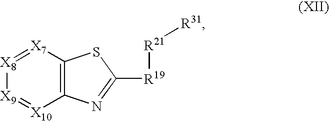

or a salt thereof, wherein:
each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

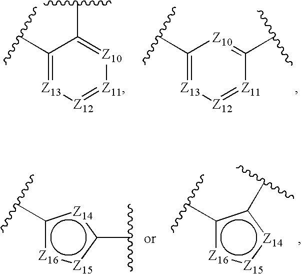

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

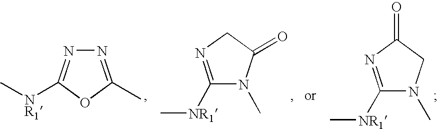

and

R³¹ is selected from an optionally substituted monocyclic or bicyclic aryl, or an optionally substituted monbcyclic or bicyclic heteroaryl,

with the proviso that when R¹⁹ is

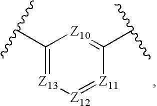

Z₁₀, Z₁₁, Z₁₂ and Z₁₃ are each CH, and R²¹ is —NHC(O)—, R³¹ is not an optionally substituted phenyl.

In certain embodiments, the compounds of Structural Formula (XI) have the following values:

each of X₇, X₈, X₉ and X₁₀ is independently selected from N, CR²⁰, or CR₁′, wherein:
each R²⁰ is independently selected from H or a solubilizing group;
each R₁′ is independently selected from H or optionally substituted C₁-C₃ straight or branched alkyl;
one of X₇, X₈, X₉ and X₁₀ is N and the others are selected from CR²⁰ or CR₁′; and
zero to one R²⁰ is a solubilizing group;

R¹⁹ is selected from:

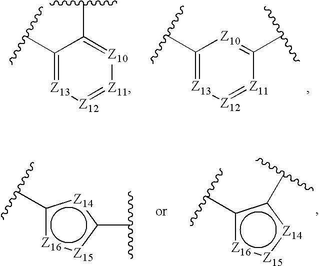

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R′₁—CR₁′R′₁—, —NR₁′—C(S)—NR₁′—CR₁′R′₁—CR₁′R′₁—, —NR₁′—C(O)—O— or —NR₁′—C(O)—CR₁′R₁′—; and

R³¹ is selected from an optionally substituted monocyclic or bicyclic aryl, or an optionally substituted monocyclic or bicyclic heteroaryl, with the proviso that:

when X₇ is N, R¹⁹ is

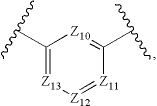

and each of Z₁₀, Z₁₁, Z₁₂ and Z₁₃ is independently selected from CR²⁰, or CR₁′, then:
a) at least one of X₈, X₉ or X₁₀ is C—(C₁-C₃ straight or branched alkyl) or C-(solubilizing group); or
b) at least one of Z₁₀, Z₁₁, Z₁₂ and Z₁₃ is CR²⁰, wherein R²⁰ is a solubilizing group.

In one embodiment, the methods disclosed herein utilize administration of a sirtuin-modulating compound of Formula (XIII):

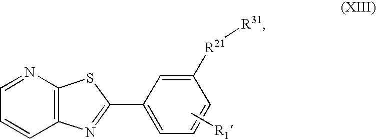

### QQ

or a salt thereof, wherein:

Rₗ′ is selected from H or optionally substituted C₁-C₃ straight or branched alkyl;

R²¹ is selected from —NRC₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁R₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

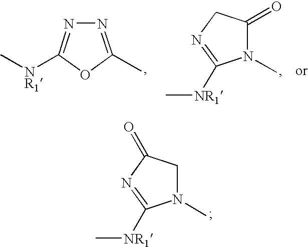

and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not unsubstituted furyl, 5-bromofuryl, unsubstituted phenyl, phenyl monosubstituted with halo or methyl, 3- or 4-methoxyphenyl, 4-butoxyphenyl, 4-t-butylphenyl, 3-trifluoromethylphenyl, 2-benzoylphenyl, 2- or 4-ethoxyphenyl, 2,3-, 2,4-, 3,4-, or 3,5-dimethoxyphenyl, 3,4,5-trimethoxyphenyl, 2,4- or 2-6 difluorophenyl, 3,4-dioxymethylene phenyl, 3,4- or 3,5-dimethlyphenyl, 2-chloro-5-bromophenyl, 2-methoxy-5-chlorophenyl, unsubstituted quinolinyl, thiazolyl substituted simultaneously with methyl and phenyl, or ethoxy-substituted pyridinyl;

when R²¹ is —NH—C(O)—CH(CH₂-CH₃)—, R³¹ is not unsubstituted phenyl;

when R²¹ is —NH—C(O)—CH₂—, R³¹ is not unsubstituted phenyl, 3-methylphenyl, 4-chlorophenyl, 4-ethoxyphenyl, 4-fluorophenyl or 4-methoxyphenyl;

when R²¹ is —NH—C(O)—CH₂—O—, R³¹ is not unsubstituted phenyl or 4-chlorophenyl; and

when R²¹ is —NH—S(O)₂—, R³¹ is not 3,4-dioxymethylene phenyl, 2,4,5-trimethylphenyl, 2,4,6-trimethylphenyl, 2,4- or 3,4-dimethylphenyl, 2,5-difluorophenyl, 2,5- or 3,4-dimethoxyphenyl, fluorophenyl, 4-chlorophenyl, 4-bromophenyl, 4-ethylphenyl, 4-methylphenyl, 3-methyl-4-methoxyphenyl, unsubstituted phenyl, unsubstituted pyridinyl, unsubstituted thienyl, chloro-substituted thienyl, or methyl-substituted benzothiazolyl.

In one embodiment, the methods disclosed herein utilize administration of a sirtuin-modulating compound of Formula (XIV):

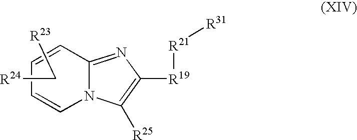

or a salt thereof, wherein:

each of R²³ and R²⁴ is independently selected from H, —CH₃ or a solubilizing group;

R²⁵ is selected from H, or a solubilizing group; and

R¹⁹ is selected from:

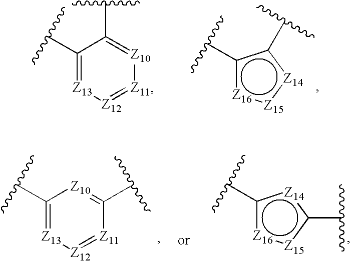

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;
each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

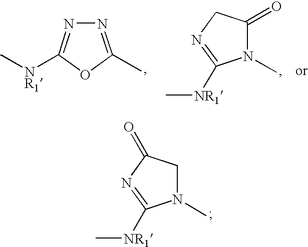

each R₁′ is independently selected from H or optionally substituted C₁-C₃ straight or branched alkyl; and

R³¹ is selected from an optionally substituted monocyclic or bicyclic aryl, or an optionally substituted monocyclic or bicyclic heteroaryl,
wherein when R¹⁹ is

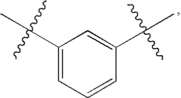

R²¹ is —NH—C(O)— and R²⁵ is —H, R³¹ is not an optionally substituted phenyl group, and wherein said compound is not 2-chloro-N-[3-[3-(cyclohexylamino)imidazo[1,2-a]pyridin-2-yl]phenyl]-4-nitrobenzamide.

In another aspect, the invention provides sirtuin-modulating compounds of Structural Formula (XV):

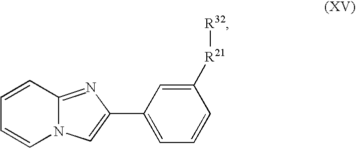

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

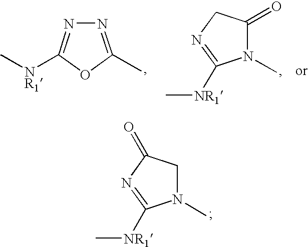

and

each R₁′ is independently selected from H or optionally substituted C₁-C₃ straight or branched alkyl; and

R³² is selected from an optionally substituted bicyclic aryl, or an optionally substituted monocyclic or bicyclic heteroaryl, wherein:

when R²¹ is —NH—C(O)—, R³² is not unsubstituted 2-furyl, 2-(3-bromofuryl), unsubstituted 2-thienyl, unsubstituted 3-pyridyl, unsubstituted 4-pyridyl,

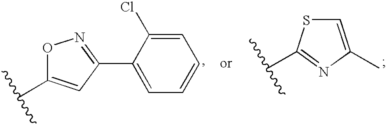

and

when R²¹ is —NR₁′—S(O)₂—, R³² is not unsubstituted 2-thienyl or unsubstituted naphthyl.

In one embodiment, the methods disclosed herein utilize administration of a sirtuin-modulating compound of Formula (XVI):

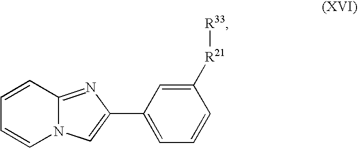

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

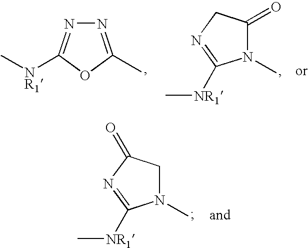

and

each R₁′ is independently selected from H or optionally substituted C₁-C₃ straight or branched alkyl; and

R³³ is an optionally substituted phenyl, wherein:

when R²¹ is —NH—C(O)—, R³³ is a substituted phenyl other than phenyl singly substituted with halo, methyl, nitro or methoxy; 2-carboxyphenyl; 4-n-pentylphenyl; 4-ethoxyphenyl; 2-carboxy-3-nitrophenyl; 2-chloro-4-nitrophenyl; 2-methoxy-5-ethylphenyl; 2,4-dimethoxyphenyl; 3,4,5-trimethoxyphenyl; 2,4 dichlorophenyl; 2,6-difluorophenyl; 3,5-dinitrophenyl; or 3,4-dimethylphenyl;

when R²¹ is —NR₁′—C(O)—CR₁′R₁′—or —NH—C(O)—CH(CH₃)—O, R³³ is a substituted phenyl;

when R²¹ is —NH—C(O)—CH₂, R³³ is not unsubstituted phenyl, 4-methoxyphenyl; 3,4-dimethoxyphenyl or 4-chlorophenyl;

when R²¹ is —NH—C(O)—CH₂—O, R³³ is not 2,4-bis(1,1-dimethylpropyl)phenyl;

when R²¹ is —NH—C(O)—NH—, R³³ is not 4-methoxyphenyl; and

when R²¹ is —NH—S(O)₂—, R³³ is a substituted phenyl other than 3-methylphenyl, 3-trifluoromethylphenyl, 2,4,5- or 2,4,6-trimethylphenyl, 2,4- or 3,4-dimethylphenyl, 2,5- or 3,4-dimethoxyphenyl, 2,5-dimethoxy-4-chlorophenyl, 3,6-dimethoxy, 4-methylphenyl, 2,5- or 3,4-dichlorophenyl, 2,5-diethoxyphenyl, 2-methyl-5-nitrophenyl, 2-ethoxy-5-bromophenyl, 2-methoxy-5-bromophenyl, 2-methoxy-3,4-dichlorophenyl, 2-methoxy-4-methyl-5-bromophenyl, 3,5-dinitro-4-methylphenyl, 3-methyl-4-methoxyphenyl, 3-nitro-4-methylphenyl, 3-methoxy-4-halophenyl, 3-methoxy-5-chlorophenyl, 4-n-butoxyphenyl, 4-halophenyl, 4-ethylphenyl, 4-methylphenyl, 4-nitrophenyl, 4-ethoxyphenyl, 4-acetylaminophenyl, 4-methoxyphenyl, 4-t-butylphenyl, or para-biphenyl.

In a further ascept, the invention provides sirtuin-modulating compounds of Structural Formula (XVII):

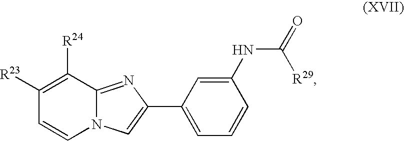

or a salt thereof, wherein:

each of R²³ and R²⁴ is independently selected from H or —CH₃, wherein at least one of R²³ and R²⁴ is H; and

R²⁹ is phenyl substituted with:

a) two —O—CH₃ groups;

b) three —O—CH₃ groups located at the 2,3 and 4 positions; or

c) one —N(CH₃)₂ group; and;

d) when R²³ is CH₃, one —O—CH₃ group at the 2 or 3 position,

wherein R²⁹ is optionally additionally substituted with a solubilizing group.

In one embodiment, the methods disclosed herein utilize administration of a sirtuin-modulating compound of Formula (XIII):

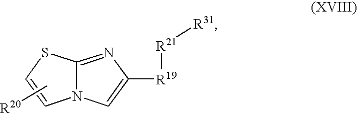

or a salt thereof, wherein

R¹⁹ is selected from:

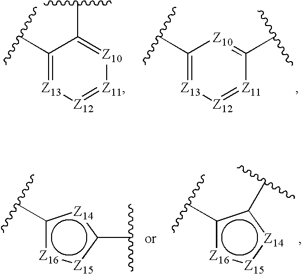

wherein:
each Z₁₀, Z₁₁, Z₁₂ and Z₁₃ is independently selected from N, CR²⁰, or CR₁′; and

each Z₁₄, Z₁₅ and Z₁₆ is independently selected from N, NR₁′, S, O, CR²⁰, or CR₁′,

wherein:

zero to two of Z₁₀, Z₁₁, Z₁₂ or Z₁₃ are N;

at least one of Z₁₄, Z₁₅ and Z₁₆ is N, NR₁′, S or O—;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group; and

zero to one R₁′ is an optionally substituted C₁-C₃ straight or branched alkyl;

each R²⁰ is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

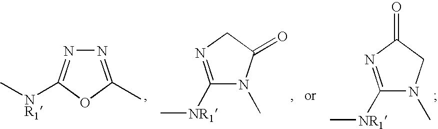

wherein each R₁′ is independently selected from H or optionally substituted C₁-C₃ straight or branched alkyl; and
R³¹ is selected from an optionally substituted monocyclic or bicyclic aryl, or an optionally substituted monocyclic or bicyclic heteroaryl, with the proviso that when R¹⁹ is

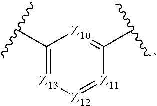

Z₁₀, Z₁₁, Z₁₂ and Z₁₃ are each CH, R²⁰ is H, and R²¹ is —NHC(O)—, R³¹ is not an optionally substituted phenyl.

In another aspect, the invention provides sirtuin-modulating compounds of Structural Formula (XX):

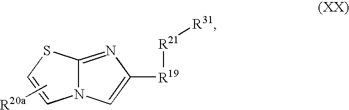

or a salt thereof, wherein

R¹⁹ is selected from:

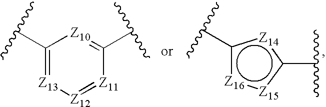

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′,

wherein:
zero to two of Z₁₀, Z₁₁, Z₁₂ or Z₁₃ are N;
at least one of Z₁₄, Z₁₅ and Z₁₆ is N, NR₁′, O or S;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group; and
zero to one R₁′ is an optionally substituted C₁-C₃ straight or branched alkyl;

each R²⁰ is independently selected from H or a solubilizing group;

R^{20a} is independently selected from H or a solubilizing group;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

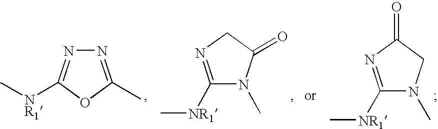

wherein
each R₁′ is independently selected from H or optionally substituted C₁-C₃ straight or branched alkyl; and k

R³¹′ is selected from an optionally substituted monocyclic or bicyclic aryl, or an optionally substituted monocyclic or bicyclic heteroaryl, wherein when R¹⁹ is

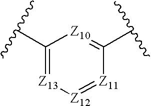

and Z₁₀, Z₁₁, Z₁₂ and Z₁₃ are each CH, R^{20a} is a solubilizing group.

In one embodiment, the methods disclosed herein utilize administration of a sirtuin-modulating compound of Formula (XXI):

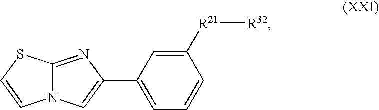

or a salt thereof, wherein

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NR₁′—CR₁′R′₁—CR₁′R′₁—, —NR₁′—C(O)—O—,

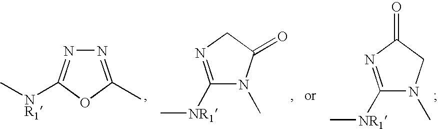

wherein
each R₁′ is independently selected from H or optionally substituted C₁-C₃ straight or branched alkyl; and

R³² is an optionally substituted monocyclic or bicyclic heteroaryl, or an optionally substituted bicyclic aryl, wherein:

when R²¹ is —NH—C(O)—CH₂—, R³² is not unsubstituted thien-2-yl;

when R² is —NH—C(O)—, R³² is not furan-2-yl, 5-bromofuran-2-yl, or 2-phenyl-4-methylthiazol-5-yl;

when R²¹ is —NH—S(O)₂—, R³² is not unsubstituted naphthyl or 5-chlorothien-2-yl.

In one embodiment, the methods disclosed herein utilize administration of a sirtuin-modulating compound of Formula (XXII):

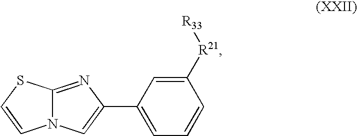

or a salt thereof, wherein:

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R₁′—, —NR₁′—C(O)—CR₁′R₁′—; —NR₁′—C(O)—CR₁′R′₁—CR₁′R′₁—, —NR₁′—C(S)—NRC₁′—CR₁′R′₁—CR₁′R′₁—, —NR₁′—C(O)—O—,

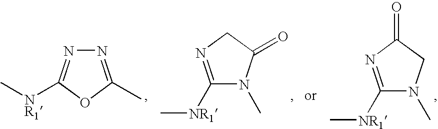

wherein each R₁′ is independently selected from H or optionally substituted C₁-C₃ straight or branched alkyl; and

R³³ is an optionally substituted phenyl, wherein:

when R²¹ is —NR₁′—C(O)—, R₁′ is not H;

when R²¹ is —NH—C(O)—CH₂ or —NH—C(O)—CH₂—O—, R³³ is not unsubstituted phenyl or 4-halophenyl; and

when R²¹ is —NH—S(O)₂—, R³³ is not unsubstituted phenyl, 2,4- or 3,4-dimethylphenyl, 2,4-dimethyl-5-methoxyphenyl, 2-methoxy-3,4-dichlorophenyl, 2-methoxy, 5-bromophenyl-3,4-dioxyethylenephenyl, 3,4-dimethoxyphenyl, 3,4-dichlorophenyl, 3,4-dimethylphenyl, 3- or 4-methylphenyl, 4-alkoxyphenyl, 4-phenoxyphenyl, 4-halophenyl, 4-biphenyl, or 4-acetylaminophenyl.

In one embodiment, the methods disclosed herein utilize administration of a sirtuin-modulating compound of Formula (XXII):

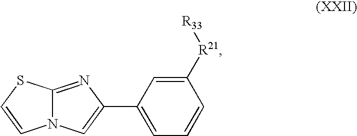

or a salt thereof wherein:

R²¹ is selected from —NH—C(O)—, or —NH—C(O)—CH₂—; and

R³³ is phenyl substituted with

a) one —N(CH₃)₂ group;

b) one CN group at the 3 position;

c) one —S(CH₃) group; or

d)

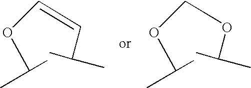

bridging the 3 and 4 positions.

In one embodiment, the methods disclosed herein utilize administration of a sirtuin-modulating compound of Formula (XXIII):

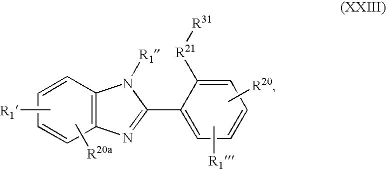

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R³¹ is not is not 3,5-dinitrophenyl, 4-

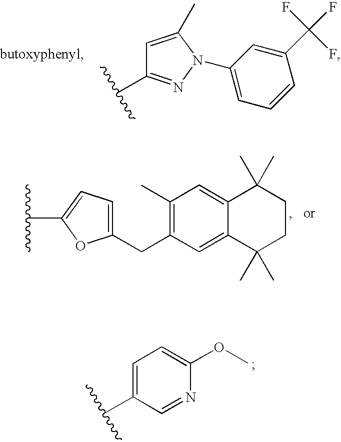

when R²¹ is —NH—C(O)— and each of R²⁰, R^{20a}, R₁′, R₁″ and R₁′″ is hydrogen, R³¹ is not

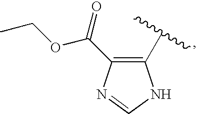

unsubstituted phenyl, 2- or 4-nitrophenyl, 2,4-dinitrophenyl, 2- or 4-chlorophenyl, 2-bromophenyl, 4-fluorophenyl, 2,4-dichlorophenyl, 2-carboxyphenyl, 2-azidophenyl, 2- or 4-aminophenyl, 2-acetamidophenyl, 4-methylphenyl, or 4-methoxyphenyl;

when R²¹ is —NH—C(O)—, R₁″ is methyl; and each of R²⁰, R^{20a}, R₁′ and R₁′″ is hydrogen, R³¹ is not 2-methylaminophenyl,

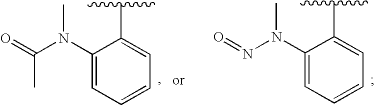

when R²¹ is —NH—C(O)—CH₂— or NH—C(S)—NH—, and each of R²⁰, R^{20a}, R₁′, R₁″ and R₁′″ is hydrogen, R³¹ is not unsubstituted phenyl;

when R²¹ is —NH—S(O)₂—, R₁″ is hydrogen or methyl, and each of R²⁰ , R^{20a}, R₁′ and R₁′″ is hydrogen, R³¹ is not 4-methylphenyl; and

when R²¹ is —NH—S(O)₂—, R^{20a} is hydrogen or —CH₂—N(CH₂CH₃)₂, and each of R²⁰, R₁′, R₁″ and R₁′″ is hydrogen, R³¹ is not

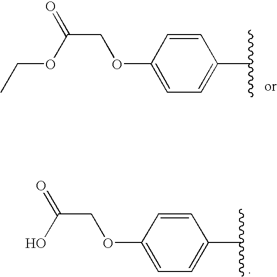

In one embodiment, the methods disclosed herein utilize administration of a sirtuin-modulating compound of Formula (XXIII):

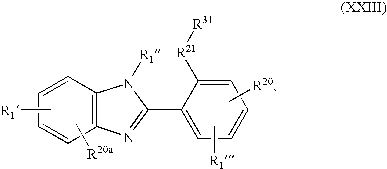

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl,

wherein:
at least one R²⁰ is a solubilizing group or at least one R₁′″ is an optionally substituted C₁-C₃ straight or branched alkyl or both; or
R^{20a} is a solubilizing group other than CH₂—N(CH₂CH₃)₂.

In one embodiment, the methods disclosed herein utilize administration of a sirtuin-modulating compound of Formula (XXIV):

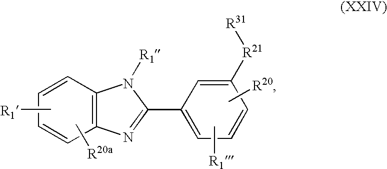

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl;

R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryi, with the provisos that:

when R²¹ is —NH—C(O)—CH₂—, R³¹ is not 2-methylphenyl, or 3,4-dimethoxyphenyl;

when R²¹ is —NH—C(O)—CH═CH—, R³¹ is not 2-chlorophenyl;

when R²¹ is —NH—C(O)—NH—, R³¹ is not unsubstituted benzimidazolyl;

when R²¹ is —NH—S(O)₂—, and each of R²⁰, R^{20a}, R₁′, R₁″ and R₁′″ is hydrogen, R³¹ is not unsubstituted phenyl, 4-chlorophenyl, 4-methylphenyl, or 4-acetoamidophenyl;

when R²¹ is —NH—S(O)₂—, each of R₁′ and R₁′″ is methyl or hydrogen, and each of R²⁰, R^{20a}, and R₁″ is hydrogen, R³¹ is not 4-nitrophenyl;

when R²¹ is —NH—C(O)—CH₂—O—, R₁′″ is methyl or hydrogen, and each of R²⁰, R^{20a}, R₁′, and R₁″ is hydrogen, R³¹ is not 2,3-, 2,5-, 2,6-, 3,4- or 3,5-dimethylphenyl, 2,4-dichloromethyl, 2,4-dimethyl-6-bromophenyl, 2- or 4-chlorophenyl, 2-(1-methylpropyl)phenyl, 5-methyl-2-(1-methylethyl)phenyl, 2- or 4-methylphenyl, 2,4-dichloro-6-methylphenyl, nitrophenyl, 2,4-dimethyl-6-nitrophenyl, 2- or 4-methoxyphenyl, 4-acetyl-2-methoxyphenyl, 4-chloro-3,5-dimethylphenyl, 3-ethylphenyl, 4-bromophenyl, 4-cyclohexyphenyl, 4-(1-methylpropyl)phenyl, 4-(1-methylethyl)phenyl, 4-(1,1 -dimethylethyl)phenyl, or unsubstituted phenyl;

when R²¹ is —NH—C(O)—CH₂—, R₁′″ is methyl or hydrogen, and each of R²⁰, R^{20a}, R₁′, and R₁″ is hydrogen, R³¹ is not unsubstituted naphthyl, 4-chlorophenyl, 4-nitrophenyl, 4-methoxyphenyl, unsubstituted phenyl, unsubstituted thienyl

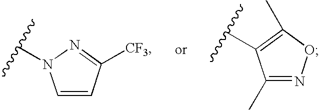

when R²¹ is —NH—C(O)—CH₂—, R₁′ is methyl, and each of R²⁰, R^{20a}, R₁″, and R₁′″ is hydrogen, R³¹ is not unsubstituted phenyl;

when R²¹ is —NH—C(O)—CH═CH, R₁′″ is methyl or hydrogen, and each of R²⁰, R^{20a}, R₁′, and R₁″ is hydrogen, R³¹ is not unsubstituted furyl, nitrophenyl-substituted furyl, 2,4-dichlorophenyl, 3,5-dichloro-2-methoxyphenyl, 3- or 4-nitrophenyl, 4-methoxyphenyl, unsubstituted phenyl, or nitro-substituted thienyl;

when R²¹ is —NH—C(O)—CH(CH₂CH₃)—, and each of R²⁰, R^{20a}, R₁′, R₁″, and R₁′″ is hydrogen, R³¹ is not unsubstituted phenyl;

when R²¹ is —NH—C(O)—CH(CH₃)—O—, R₁′″ is methyl or hydrogen, and each of R²⁰, R^{20a}, R₁′, and R₁′″ is hydrogen, R³¹ is not 2,4-dichlorophenyl.

In one embodiment, the methods disclosed herein utilize administration of a sirtuin-modulating compound of Formula (XXIV):

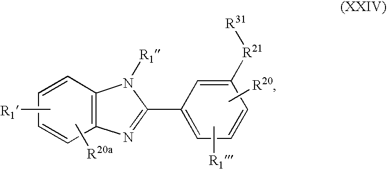

or a salt thereof, wherein:

each R and R is independently selected from H or a solubilizing group and at least one of R²⁰ and R^{20a} is a solubilizing group;

each R₁′, R₁″ and R₁′″ is independently selected from H or optionally substituted C₁-C₃ straight or branched alkyl;

R²¹ is selected from —NR²³—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—, wherein R²³ is an optionally substituted C₁-C₃ straight or branched alkyl; and

R³¹ is selected from an optionally substituted monocyclic or bicyclic aryl, or an optionally substituted monocyclic or bicyclic heteroaryl.

In one embodiment, the methods disclosed herein utilize administration of a sirtuin-modulating compound of Formula (XXV):

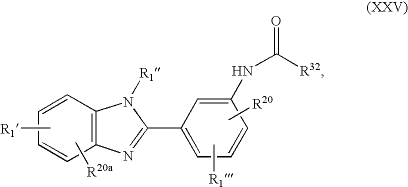

or a salt thereof, wherein:

each R²⁰ and R^{20a} is independently selected from H or a solubilizing group, wherein at least one of R²⁰ and R^{20a} is a solubilizing group;

each R₁′, R₁″ and R₁′″ is independently selected from H or optionally substituted C₁-C₃ straight or branched alkyl; and

R³² is an optionally substituted phenyl.

In one embodiment, the methods disclosed herein utilize administration of a sirtuin-modulating compound of Formula (XXVI):

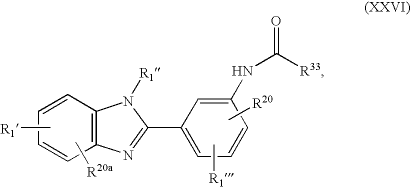

or a salt thereof, wherein:

each R²⁰ and R^{20a} is independently selected from H or a solubilizing group;

each R₁′, R₁″ and R₁′″ is independently selected from H or optionally substituted C₁-C₃ straight or branched alkyl; and

R³³ is selected from an optionally substituted heteroaryl or an optionally substituted bicyclic aryl, with the provisos that:

when each of R₁′ and R₁′″ is hydrogen or methyl and each of R₁″, R₂₀ and R^{20a} is hydrogen, R³³ is not 5,6,7,8-tetrahydronaphthyl, unsubstituted benzofuryl, unsubstituted benzothiazolyl, chloro- or nitro-substituted benzothienyl, unsubstituted furyl, phenyl-, bromo- or nitro-substituted furyl, dimethyl-substituted isoxazolyl, unsubstituted naphthyl, 5-bromonaphthyl, 4-methylnaphthyl, 1- or 3-methoxynaphthyl, azo-substituted naphthyl, unsubstituted pyrazinyl, S-methyl-substituted pyridyl, unsubstituted pyridyl, thienyl- or phenyl-substituted quinolinyl, chloro-, bromo- or nitro-substituted thienyl, unsubstituted thienyl, or

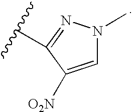

In one embodiment, the methods disclosed herein utilize administration of a sirtuin-modulating compound of Formula (XXVI):

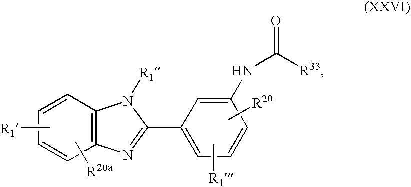

or a salt thereof, wherein:

each R²⁰ and R^{20a} is independently selected from H or a solubilizing group, wherein at least one of R²⁰ or R^{20a} is a solubilizing group;

each R₁′, R₁″ and R₁′″ is independently selected from H or optionally substituted C₁-C₃ straight or branched alkyl; and

R³³ is selected from an optionally substituted heteroaryl or an optionally substituted bicyclic aryl.

In one embodiment, the methods disclosed herein utilize administration of a sirtuin-modulating compound of Formula (XXVII):

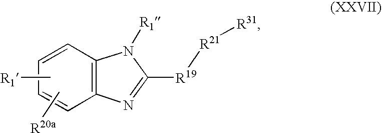

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

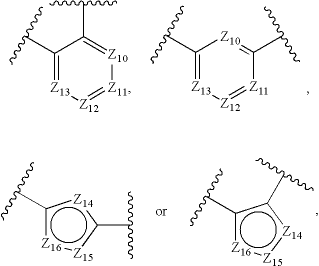

wherein:
each Z₁₀, Z₁₁, Z₁₂ and Z₁₃ is independently selected from N, CR²⁰, or CR₁′; and
each Z₁₄, Z₁₅ and Z₁₆ is independently selected from N, NR₁′, S, O, CR²⁰, or CR₁′, wherein:
zero to two of Z₁₀, Z₁₁, Z₁₂ or Z₁₃ are N;
at least one of Z₁₄, Z₁₅ and Z₁₆ is N, NR₁′, S or O;
zero to one of Z₁₄, Z₁₅ and Z₁₆ is S or O;
zero to two of Z₁₄, Z₁₅ and Z₁₆ are N or NR₁′;
zero to one R²⁰ is a solubilizing group;
zero to one R₁′ is an optionally substituted C₁-C₃ straight or branched alkyl; and
R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R₁′—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R₁′—C(O)—NR₁′—, —CR₁′R₁′—C(O)—NR₁′—, —NR₁′—C(O)—CR₁′═CR₁′—CR₁′R₁′—, —NR₁′—C(═N—CN)—NR₁′—, —NR₁′—C(O)—CR₁′R₁′—O—, —NR₁′—C(O)—CR₁′R₁′—CR₁′R₁′—O—, —NR₁′—S(O)₂—CR₁′R₁′—, —NR₁′—S(O)₂—CR₁′R₁′—CR₁′R′₁—, or —NR₁′—C(O)—CR₁′R₁′—; and

R³¹ is selected from an optionally substituted monocyclic or bicyclic aryl, or an optionally substituted monocyclic or bicyclic heteroaryl,
provided that when R²¹ is —NH—C(O)— and R¹⁹ is

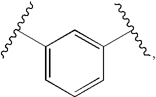

R³¹ is not unsubstituted pyridyl, 2,6-dimethoxyphenyl, 3,4,5-trimethoxyphenyl or unsubstituted furyl.

In a particular aspect, the invention provides sirtuin-modulating compounds of Structural Formula (XXVII):

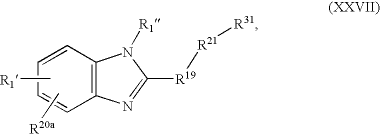

or a salt thereof, wherein:
each R²⁰ and R^{20a} is independently selected from H or a solubilizing group;
each R₁′ and R₁″ is independently selected from H or optionally substituted C₁-C₃ straight or branched alkyl;

R¹⁹ is selected from:

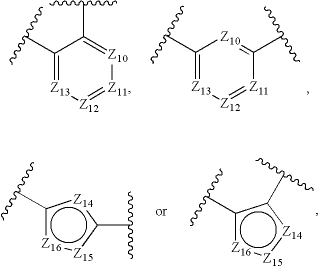

wherein:

each Z₁₀, Z₁₁, Z₁₂ and Z₁₃ is independently selected from N, CR²⁰, or CR₁′; and

each Z₁₄, Z₁₅ and Z₁₆ is independently selected from N, NR₁′, S, O, CR²⁰, or CR₁′, wherein:

zero to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group;

zero to one R₁′ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —C(O)—NR₁′—, —C(O)—NR₁′—S(O)₂—, —NR₁′—, —CR₁′R′₁—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R¹⁹ is not pyrazolyl;

when R²¹ is —NH—, and R¹⁹ is thiazolyl, R³¹ is not optionally substituted phenyl or optionally substituted pyridyl;

when R²¹ is —NH—C(O)—CH₂—, and R¹⁹ is pyrazolyl, R³¹ is not unsubstituted indolyl or unsubstituted phenyl;

when R²¹ is —NH—C(O)—CH₂—, and R¹⁹ is

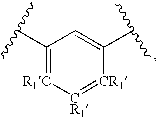

R³¹ is not 2-methylphenyl or 3,4-dimethoxyphenyl;

when R²¹ is —NH—C(O)—CH═CH—, and R¹⁹ is

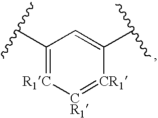

R³¹ is not 2-chlorophenyl;

when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl, 2,5-dimethylphenyl, 3,4-dichlorophenyl, or 4-chlorophenyl;

when R²¹ is —NH—C(O)—NH—, and R¹⁹ is

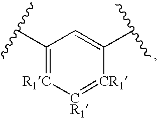

R³¹ is not unsubstituted benzimidazolyl;

when R²¹ is —NH—, and R¹⁹ is pyrazolyl, R³¹ is not unsubstituted pyridyl;

when R^{20a} is a solubilizing group, R¹⁹ is 1-methylpyrrolyl and R²¹ is —NH—C(O)—, R³¹ is not unsubstituted phenyl, unsubstituted furyl, unsubstituted pyrrolyl, unsubstituted pyrazolyl, unsubstituted isoquinolinyl, unsubstituted benzothienyl, chloro-substituted benzothienyl, 2-fluoro-4-chlorophenyl or phenyl singly substituted with a solubilizing group;

when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1-methyl-4-(1,1-dimethylethyloxycarbonylamino)pyrrol-2-yl or phenyl singly substituted with a solubilizing group;

when R²¹ is —NH— and R¹⁹ is pyridyl, oxadiazolyl or thiadiazolyl, R³¹ is not unsubstituted phenyl, 3-methoxyphenyl or 4-methoxyphenyl;

when R²¹ is —NH—C(O)— and R¹⁹ is thiazolyl or pyrimidinyl, R³¹ is not unsubstituted phenyl;

when R²¹ is —NH—C(O)— and R¹⁹ is

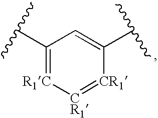

R³¹ is not unsubstituted pyridyl, unsubstituted thienyl, unsubstituted phenyl, 2-methylphenyl, 4-fluorophenyl, 4-methoxyphenyl, 4-methylphenyl, 3,4-dioxyethylenephenyl, 3-acetylamino-4-methylphenyl, 3-[(6-amino-1-oxohexyl)amino]-4-methylphenyl, 3-amino-4-methylphenyl, 2,6-dimethoxyphenyl, 3,5-dimethoxyphenyl, 3-halo-4-methoxyphenyl, 3-nitro-4-methylphenyl, 4-propoxyphenyl, 3,4,5-trimethoxyphenyl or unsubstituted furyl;

when R²¹ is —NH—C(O)— and R¹⁹ is

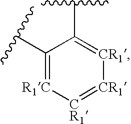

R³¹ is not 3,5-dinitrophenyl, 4-butoxyphenyl

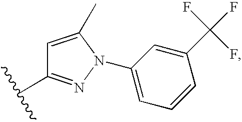

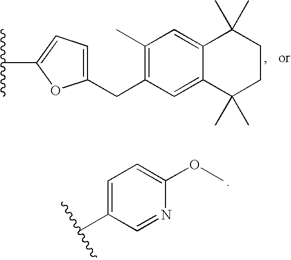

In a more particular embodiment, the invention provides sirtuin-modulating compounds of Structural Formula (XXVII):

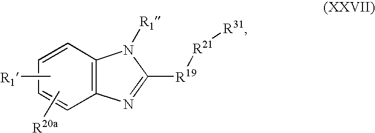

or a salt thereof, wherein:

each R²⁰ and R^{20a} is independently selected from H or a solubilizing group;

each R₁′ and R₁″ is independently selected from H or optionally substituted C₁-C₃ straight or branched alkyl;

R¹⁹ is selected from:

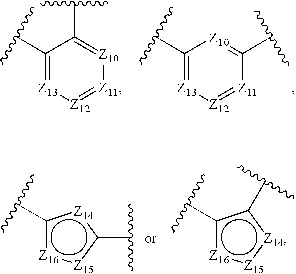

wherein:
each Z₁₀, Z₁₁, Z₁₂ and Z₁₃ is independently selected from N, CR²⁰, or CR₁′; and

each Z₁₄, Z₁₅ and Z₁₆ is independently selected from N, NR₁′, S, O, CR , or CR₁′, wherein:

one to two of Z₁₀, Z₁₁, Z₁₂ or Z₁₃ are N;

at least one of Z₁₄, Z₁₅ and Z₁₆ is N, NR₁′, S or O;

zero to one of Z₁₄, Z₁₅ and Z₁₆ is S or O;

zero to two of Z₁₄, Z₁₅ and Z₁₆ are N or NR₁′;

zero to one R²⁰ is a solubilizing group;

zero to one R₁′″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl, with the provisos that:

when R²¹ is —NH—C(O)—, R¹⁹ is not pyrazolyl;

when R²¹ is —NH—C(O)—CH₂—, and R¹⁹ is pyrazolyl, R³¹ is not unsubstituted indolyl or unsubstituted phenyl;

when R²¹ is —NH—C(O)—NH—, and R¹⁹ is pyrazolyl, R³¹ is not unsubstituted isoxazolyl, unsubstituted naphthyl, unsubstituted phenyl, 2,6-difluorophenyl; 2,5-dimethylphenyl; 3,4-dichlorophenyl; or 4-chlorophenyl;

when R^{20a} is a solubilizing group, R¹⁹ is 1-methylpyrrolyl and R²¹ is —NH—C (O)—, R³¹ is not unsubstituted phenyl; unsubstituted furyl; unsubstituted pyrrolyl; unsubstituted pyrazolyl; unsubstituted isoquinolinyl; unsubstituted benzothienyl; chloro-substituted benzothienyl; 2-fluoro-4-chlorophenyl or phenyl singly substituted with a solubilizing group;

when R^{20a} is a solubilizing group, R¹⁹ is thienyl and R²¹ is —NH—C(O)—, R³¹ is not unsubstituted phenyl;

when R^{20a} is a solubilizing group, R¹⁹ is methylimidazolyl and R²¹ is —NH—C(O)—, R³¹ is not 1 -methyl-4-(1,1 -dimethylethyloxycarbonylamino)pyrrol-2-yl or phenyl singly substituted with a solubilizing group; and

when R²¹ is —NH—C(O)— and R¹⁹ is thiazolyl or pyrimidinyl, R³¹ is not unsubstituted phenyl.

In one embodiment, the methods disclosed herein utilize administration of a sirtuin-modulating compound of Formula (XXVIII):

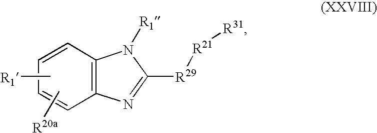

or a salt thereof, wherein:

each R²⁰ and R^{20a} is independently selected from H or a solubilizing group;

Sirt1 gene contains a polymorphic variant that is associated with a risk of having or developing a Sirt1 mediated disease or disorder. The kit may further comprise instructions for use in diagnosing a subject as having, or having a predisposition, towards developing a Sirt1 mediated disease or disorder. The probe or primers of the kit can be a probe or primer that binds to SEQ ID NO: 1, or a sequence complementary thereto. Such probe or primers may bind, for example, at and/or flanking a polymorphic site of Sirt1, such as the sites set forth in Table 1 or as described herein above.

Kits for amplifying a region of a gene comprising a polymorphic variant of Sirt1 of interest may comprise one, two or more primers.

In an exemplary embodiment, a kit may comprise a microarray suitable for detection of a variety of Sirt1 polymorphic variants. Examples of such microarrays are described further herein above.

In other embodiments, the kits provided herein may comprise one or more antibodies that are capable of specifically recognizing a polypeptide variant of Sirt1 arising from a polymorphic variant of a Sirt1 nucleic acid sequence. In an exemplary embodiment, the kit may include a panel of antibodies able to specifically bind to a variety of polypeptide variants of Sirt1 encoded by polymorphic variants of Sirt1 nucleic acid sequences. The kits may further comprise additional components such as substrates for an enzymatic reaction. The antibodies may be used for research, diagnostic, and/or therapeutic purposes.

In yet other embodiments, the kits provided herein may comprise reagents for detecting Sirt1 deacetylase activity. For example, the kits may comprise a Sirt1 substrate, buffers, detection reagents, etc.

In other embodiments, methods for identifying sirtuin modulating compounds are provided. The methods may involve for example, correlating the presence or absence of a Sirt1 polymorphic variant with the activity or efficacy of a sirtuin modulating compound. Such methods may be carried out using in vitro or in vivo methods for determining Sirt1 activity and/or efficacy.

Intact cells or whole animals expressing polymorphic variants of Sirt1 can be used in screening methods to identify candidate drugs. For example, a permanent cell line may be established from an individual exhibiting one or more polymorphic variants of Sirt1. Alternatively, cells (including without limitation mammalian, insect, yeast, or bacterial cells) may be programmed to express a gene comprising one or more Sirt1 sequences having polymorphic variants by introduction of appropriate DNA into the cells. Identification of candidate sirtuin modulating compounds can be achieved using any suitable Sirt1 deacetylase assay. A variety of assays are known in the art or are commercially available. Examplary sirtuin deacetylase assays are described herein below. Such assays may include without limitation (i) assays that measure selective binding of test compounds to particular polypeptide variants of Sirt1 encoded by Sirt1 gene sequences having polymorphic variants; (ii) assays that measure the ability of a test compound to modify (i.e., inhibit or enhance) a measurable activity or function of polypeptide variants of Sirt1 encoded by Sirt1 gene sequences having polymorphic variants; and (iii) assays that measure the ability of a compound to modify (i.e., inhibit or enhance) the transcriptional activity of sequences derived from the promoter (i.e., regulatory) region of a Sirt1 gene sequence having at least one polymorphic variant in the regulatory region.

In other embodiments, transgenic animals are created in which (i) one or more human Sirt1 genes, having different sequences at particular polymorphic sites are stably inserted into the genome of the transgenic animal; and/or (ii) the endogenous Sirt1 gene may be inactivated and replaced with human Sirt1 genes having different sequences at particular polymorphic sites. See, e.g., Coffman, Semin. Nephrol. 17:404, 1997; Esther et al., Lab. Invest. 74:953, 1996; Murakarni et al., Blood Press. Suppl. 2:36, 1996. Such animals can be treated with candidate compounds and monitored, for example, for one or more clinical markers of disease, expression levels (mRNA and/or protein) of Sirt1, activity or Sirt1, etc.

### EXEMPLIFICATION

The invention now being generally described, it will be more readily understood by reference to the following examples which are included merely for purposes of illustration of certain aspects and embodiments of the present invention, and are not intended to limit the invention in any way.

### Example 1

### Genetic Analysis of SIRT1

each R₁′ and R₁″ is independently selected from H or optionally substituted C₁-C₃ straight or branched alkyl;

R²⁹ is selected from:

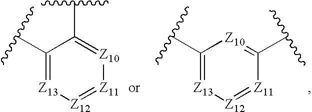

wherein:

each Z₁₀, Z₁₁, Z₁₂ and Z₁₃ is independently selected from N, CR²⁰, or CR₁′, wherein one of Z₁₀, Z₁₁, Z₁₂ or Z₁₃ is N; and

zero to one R²⁰ is a solubilizing group;

zero to one R₁′″ is an optionally substituted C₁-C₃ straight or branched alkyl; and

R²¹ is selected from —NR₁′—C(O)—, —NR₁′—S(O)₂—, —NR₁′—C(O)—NR₁′—, —NR₁′—C(S)—NR₁′—, —NR₁′—C(S)—NR₁′—CR₁′R′₁—, —NR₁′—C(O)—CR₁′R₁′—NR₁′—, —NR₁′—C(═NR₁′)—NR₁′—, —NR₁′—C(O)—CR₁′═CR₁′—, —NR₁′—S(O)₂—NR₁′—, —NR₁′—C(O)—NR₁′—S(O)₂—, —NR₁′—CR₁′R′₁—C(O)—NR₁′—, —NR₁′—C(O)—CR₁′═CR₁′—CR₁′R₁′—, —NR₁′—C(═N—CN)—NR₁′—, —NR₁′—C(O)—CR₁′R′₁—O—, —NR₁′—C(O)—CR₁′R₁′—CR₁′R₁′—O—, —NR₁′—S(O)₂—CR₁′R′₁—, —NR₁′—S(O)₂—CR₁′R₁′—CR₁′R₁′—, or —NR₁′—C(O)—CR₁′R₁′—; and

R³¹ is selected from an optionally substituted monocyclic or bicyclic aryl, or an optionally substituted monocyclic or bicyclic heteroaryl.

The methods disclosed herein may also utlize pharmaceutical compositions comprising one or more compounds of Formulas (I)-(XXVIII) or a salt, prodrug or metabolite thereof.

### 7. Kits and Screening Assays

Provided herein are kits that may be used to to determine the presence or absence of one or more polymorphic variants of Sirt1. Such kits may be used to diagnose, or predict a subject's susceptibility to, a Sirt1 mediated disease or disorder. This information could then be used, for example, to optimize treatment with a sirtuin modulating compound for subjects having one or more polymorphic variants.

In preferred embodiments, the kit comprises a probe or primer which is capable of hybridizing to a polymorphic variant of a Sirt1 gene thereby determining whether the

The following example describes a clinical genetic study designed to look at whether the genetic variation in the human SIRT1 gene is associated with exercise endurance. The collection of subjects and the study protocol have been published (Salmenniemi, U., Ruotsalainen, E., Pihlajamaki, J., Vauhkonen, I., Kainulainen, S. et al (2004). “Multiple abnormalities in glucose and energy metabolism and coordinated changes in levels of adiponectin, cytokines, and adhesion molecules in subjects with metabolic syndrome.” Circulation 110, 3842-3848) and a brief summary is available online. The study protocol was approved by the Ethics Committee of the University of Kuopio and all subjects gave an informed consent. The mean age and BMI of the-subjects was 34 years and 23 kg/m², respectively. All subjects underwent an OGTT. Indirect calorimetry was performed in the fasting state and during hyperinsulinemia (40 mU/m²/min insulin infusion for 120 min) as described (Salmenniemi et al., 2004). The rates of energy expenditure were calculated according to Ferrannini et al. (Ferrannini, E., Buzzigoli, G., Bevilacqua, S., Boni, C., Del Chiaro, D. et al (1988). “Interaction of carnitine with insulin-stimulated glucose metabolism in humans.” Am. J. Physiol 255, E946-E952). Selection of the SNPs of Sirt1 was based on linkage disequilibrium and haplotype block analysis of the HapMap project data (http://www.hapmap.org; Public Release #20/Phase II, Jan. 24, 2006; population: Utah residents with ancestry from northern and western Europe).

Subjects and Indirect calorimetry: The collection of subjects and the study protocol have been previously published (Salmenniemi et al., 2004). In brief, the subjects were selected from an ongoing study and included healthy normal weight (body mass index <26.0 kg/ m²) non-diabetic offspring of patients with type 2 diabetes. The diabetic patients (probands) were randomly selected among type 2 diabetic subjects living in the region of the Kuopio. Spouses of the probands had to have a normal glucose tolerance in an oral glucose tolerance test (OGTT). A total of 123 offspring (1-3 from each family) were studied. The study protocol was approved by the Ethics Committee of the University of Kuopio. All study subjects gave an informed consent. The mean age and body mass index of the subjects was 34 years and 23 kg/m², respectively. All subjects underwent an OGTT. Furthermore, indirect calorimetry was performed in the fasting state and during hyperinsulinemia (40 mU/ m²/min insulin infusion for 120 minutes) as described (Salmenniemi et al., 2004). Indirect calorimetry was performed with a computerized flow-through canopy gas analyzer system (Dealtatract; Datex, Helsinki, Finland). The first 10 min of each measurement was discarded and the mean value of the last 20 min was used in calculations.

Genotyping and Data Analysis: Selection of the single nucleotide polymorphisms (SNPs) of Sirt1 was based on linkage disequilibrium and haplotype block analysis of the HapMap project data (http://www.hapmap.org; Public Release #20/Phase II, Jan. 24, 2006; population: Utah residents with ancestry from northern and western Europe). Haploview software (http://www.broad.mit.edu/mpg/haploview/), was used to analyze the HapMap data from the region of the Sirt1 gene locus (33.2 kb upstream, 33.7 kb the Sirt1 gene and 33.2 kb downstream). Five SNPs (rs12778366 (promoter C/T), rs3740051 (promoter A/G), rs2236319 (intron 3 A/G), rs2272773 (L332L, C/T), rs10997870 (intron 6 G/T) were selected to represent different haplotype blocks. According to Tagger analysis (http://www.broad.mit.edu/mpg/tagger/) selected SNPs capture 91.7% of common variants (minor allele frequency >5%). Genotyping success rate of eight SNPs was 100%. Genotyping of SNPs was performed with the TaqMan Allelic Discrimination Assays (Applied Biosystems). Genotyping reaction was amplified on a GeneAmp PCR system 2700/2720 (95° C. for 10 min, followed by 40 cycles of 95° C. 15 s and 60° C. 1 min), and fluorescence was detected on an ABI Prism 7000 Sequence Detection System (Applied Biosystems). All the SNPs followed the Hardy-Weinberg expectations. Data analysis was carried out with the SPSS 11.0 for windows programs. The results for continuous variables are given as means ±SD. Linear mixed model analysis was applied to adjust for confounding factors. For mixed model analysis we included the pedigree (coded as a family number) as a random factor, the Sirt1 genotype and gender as fixed factors, and age as a covariate. All results were analyzed according to the dominant model.

### Results

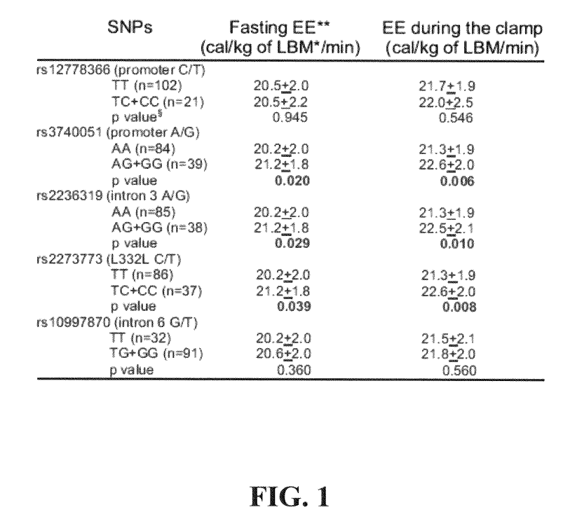

To determine whether common alleles in Sirt1 might contribute to heritable phenotypic variation in EE in humans, we investigated the effects of 5 genetic variants in the Sirt1 gene on EE as profiled in a cohort of healthy, normal weight (body mass index <26.0 kg/m2), non-diabetic offspring of type 2 diabetic patients (Ferrannini et al., 1988). Three out of 5 SNPs tested, ie. rs3740051 (promoter A/G), rs2236319 (intron 3 A/G) and rs 2273773 (L332L C/T), were significantly associated with whole body EE as evaluated either during fasting or during a hyperinsulinemic clamp (FIG. 1). In addition, these same three SNPs were also significantly associated with insulin sensitivity. These data indicate that in humans, Sirt1 genetic polymorphisms, co-vary with the degree of EE and insulin sensitivity, which provides an independent genetic argument that bolsters the direct involvement of SIRT1 in modulating EE, insulin sensitivity and other aspects of diabetes and metabolic disease.

### Example 2

### Synthesis of the Sirt1 Activator

### Preparation of 6-(2-Nitro-phenyl)-imidazo[2,1-b]thiazole-3-carboxylic acid ethyl ester

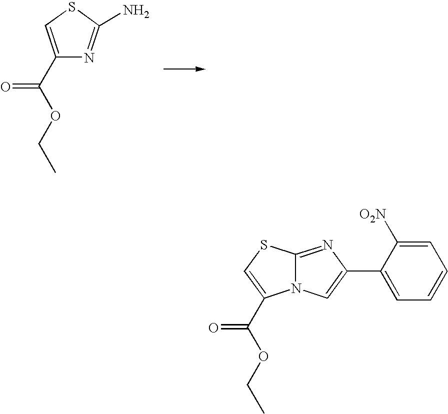

In a typical run, ethyl 2- aminothiazole-4-carboxylate (2.1 g, 0.0123 mol) was taken up in methyl ethyl ketone (25 mL) along with 2-bromo-2′—nitroacetophenone (3.0 g, 0.0123 mol). The reaction mixture was stirred under reflux for 18 hours. It was then cooled to room temperature and filtered to remove some of the solids. The filtrate was concentrated to afford 3.10 g of 6-(2-nitro-phenyl)-imidazo[2,1-b]thiazole-3-carboxylic acid ethyl ester (Calc'd for C₁₄H₁₂N₃O₄S: 318.3 , [M+H]+ found: 319).

### Preparation of [6-(2-nitro-phenyl)-imidazo[2,1-b]thiazol-3-yl]-methanol

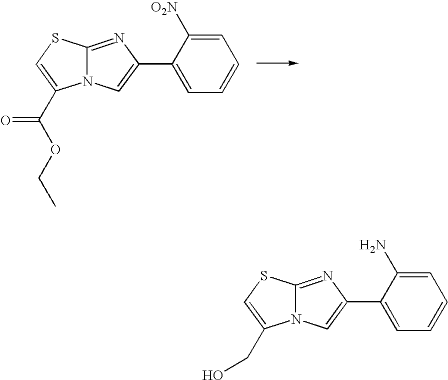

6-(2-Nitro-phenyl)-imidazo[2,1-b]thiazole-3-carboxylic acid ethyl ester (14.50 g, 0.0458 mol) was taken up in THF (100 mL) and water (100 mL) containing NaOH (7.3 g, 4 eq). The reaction mixture was stirred at room temperature for 18 hours. It was then concentrated. The aqueous layer was washed once with CH₂Cl₂ and then acidified with 6 N HCl. The solids were collected by filtration and dried to afford 7.4 g of the acid intermediate. This material (7.4 g, 0.0256 mol) was taken up in anhydrous THF (200 mL) along with N-methylmorpholine (2.8 mL, 0.0256 mol) and cooled to 0°. Isobutyl chloroformate (3.35 mL, 0.0256 mol) was added and the reaction mixture was stirred in the ice bath for 3 hours. NaBH4 (0.97 g, 0.0256 mol) was added as a solution in water (30 mL). The reaction mixture was stirred at 0° for 45 min. It was then warmed to room temperature and concentrated. The aqueous layer was extracted with CH₂Cl₂. The combined organic layers were dried (Na₂SO₄) and concentrated to afford the crude product. Purification by chromatography (Isco, using a mixture of pentane/EtOAc) afforded 5.20 g of [6-(2-nitro-phenyl)-imidazo[2,1-b]thiazol-3-yl]-methanol (74% yield) (Calc'd for C₁₂H₁₁N₃OS: 245.3, [M+H]+ found: 246).

### Preparation of 4-[6-(2-amino-phenyl)-imidazo[2,1-b]thiazol-3-ylmethyl]-piperazine-1-carboxylic acid tert-butyl ester

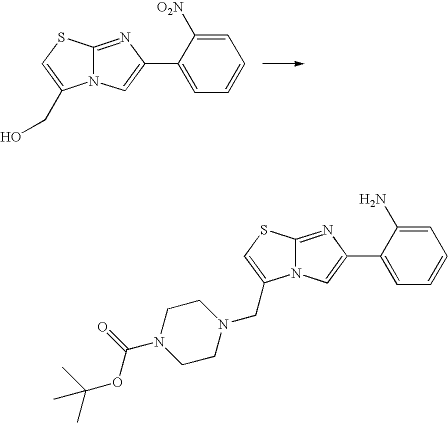

[6-(2-Nitro-phenyl)-imidazo[2,1-b]thiazol-3-yl]-methanol (1.0 g, 3.64 mmol) was dissolved in CH₂Cl₂ (100 mL) along with Et₃N (0.51 mL, 3.64 mmol). Methanesulfonyl chloride (1 eq, 0.28 mL) was added and the reaction mixture was warmed to room temperature and stirred for 15 min. It was then quenched with brine and extracted with CH₂Cl₂. The combined organic layers were dried (Na₂SO₄) and concentrated to afford the mesylate intermediate. This material was taken up CH₃CN (4 mL) along with Et₃N (0.51 mL, 3.64 mmol) and Boc-piperazine (680 mg, 3.64 mmol) and stirred at room temperature for 1 day. The reaction mixture was concentrated and the resulting residue was partitioned between CH₂Cl₂ and water. The organic layer was dried (Na₂SO₄) and concentrated to afford essentially quantitative yield of the product. This material was taken up in MeOH (6 mL) and water (1 mL) along with sodium hydrosulfide hydrate (200 mg). The resulting reaction mixture was stirred under reflux for 24 hours. It was then cooled to room temperature and concentrated. The resulting residue was diluted with water (2 mL) and extracted with CH₂Cl₂. The combined organic layers were dried (Na₂SO₄) and concentrated to afford 0.90 g of 4-[6-(2-amino-phenyl)-imidazo[2,1-b]thiazol-3-ylmethyl]-piperazine-1-carboxylic acid tert-butyl ester (Calc'd for C₂₁H₂₇N₅O₂S: 413.5, [M+H]+ found: 414).

### Preparation of SRT1933:

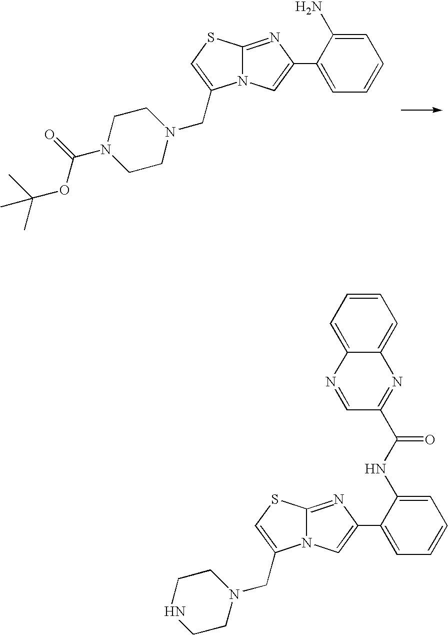

4-[6-(2-Amino-phenyl)-imidazo[2,1 -b]thiazol-3-ylmethyl]-piperazine-1-carboxylic acid tert-butyl ester (0.25 mmol) was taken up in 1 mL of pyridine along with 1 eq (50 mg) of 2-quinoxaloyl chloride. The reaction mixture was heated in a Biotage microwave reactor (160°×10 min). It was then cooled to room temperature and concentrated. The resulting crude product was purified by chromatography (Isco, gradient elution, CH₂Cl₂ to 95% CH₂Cl₂, 4% MeOH and 1% Et₃N). The purified product was then treated with a solution containing 25% TFA in CH₂Cl₂ (2mL) for 2 hours. It was then concentrated and the resulting residue was triturated with Et₂O to afford the desired product as the TFA salt (Calc'd for C₂₅H₂₃N₇OS: 469.5, [M+H]+ found: 470). ¹HNMR (300 MHz, DMSO-d₆) δ: 13.9 (brs, 1 H), 9.8 (brs, 1 H), 9.6 (brs, 1 H) 8.9-7.2 (m, 11 H), 4.8 (br s, 2 H). The analytical HPLC was performed on an Agilent 1100 Series HPLC equipped with a 3.5 um Eclipse XDB-C18 (4.6 mm×100 mm) column with the following conditions: MeCN/H₂O, modified with 0.1 % Formic acid mobile phase. Gradient elution: 5% hold (2 min), 5% to 95% gradient (11 min), 95% to 5% gradient (0.3 min), 5% hold (2.7 min), 15 min. total run time. Flow rate: 0.8 ml/min. Retention time=3.04 min.

### Example 3

### Analysis of Sirtuin Activity

The following example describes methods for the identification and characterization of Sirt1 activators. Human SIRT1 is expressed from the pSIRT1FL vector which places expression under the control of the T7 promoter. The protein was expressed in E. coli BL21(DE3)Star as an N-terminal fusion to a hexa-histidine affinity tag. The expressed protein was purified by Ni²⁺-chelate chromatography. The eluted protein was then purified by size exclusion chromatography followed by ion exchange. The resulting protein was typically >95% pure as assessed by SDS-PAGE analysis. The mass spectrometry based assay utilizes a peptide having 20 amino acid residues as follows: Ac-Glu-Glu-Lys(Biotin)-Gly-Gln-Ser-Thr-Ser-Ser-His-Ser-Lys(Ac)-Nle-Ser-Thr-Glu-Gly-Lys(5TMR)-Glu-Glu-NH2 (SEQ ID NO:2) wherein K(Ac) is an acetylated lysine residue and Nle is a norleucine. The peptide is labeled with the fluorophore 5TMR (excitation 540 nm/emission 580 nm) at the C-terminus for use in the FP assay described above. The sequence of the peptide substrate is based on p53 with several modifications.

The mass spectrometry assay was conducted as follows: 0.5 μM peptide substrate and 120 μM βNAD⁺ was incubated with 10 nM SIRT1 for 25 minutes at 25° C. in a reaction buffer (50 mM Tris-acetate pH 8, 137 mM NaCl, 2.7 mM KCl, 1 mM MgCl₂, 5 mM DTT, 0.05% BSA). Test compounds were added to the reaction or vehicle control, DMSO. After the incubation with SIRT1, 10% formic acid was added to stop the reaction. Determination of the mass of the substrate peptide allows for precise determination of the degree of acetylation (i.e. starting material) as compared to deacetylated peptide (product).

### Results

The activity of an exemplary compound against SIRT1 enzyme was profiled using a mass spectrometry readout. In this assay the conversion of acetylated peptide substrate to deacetylated peptide product is tracked by monitoring the change in mass (44 AMU) upon loss of the acetyl group. Potency was tracked by determining the concentration of compound required to increase enzyme activity by 50% (EC_{1.5}) and the % maximum activation achieved at the highest doses of compound tested. Based on this assay, SRT1933 has an EC_{1.5} of 0.16 μM and a maximum activation of greater than 900%.

### Example 4

### Mass Spectrometry Analysis of Sirtuin Activity

The following example describes an alternative mass spec based assay for determination of Sirt1 deacetylase activity. Instead of relying on purified or recombinant enzyme, the reaction utilizes endogenous Sirt1 enzyme from cell or tissue extracts. This allows for the determination of endogenous sirtuin activity. For tissues, cells or samples of human origin, the Sirt1 haplotype can also be determined and correlated with Sirt1 enzymatic activity. The cells or tissues can be pretreated with Sirt1 modulators or other control compounds either following isolation or following pharmacological intervention in vivo. Alternatively, this measurement of endogenous sirtuin activity can be measured in various clinical samples following physiological manipulation (diet, exercise, age, disease progression, etc.) or following pharmacological intervention including studies designed to study dose responsiveness and escalation, vehicle or placebo control, dosing regimen, drug combination and synergy, etc.

This example describes the procedure for isolating viable (living) white blood cells (WBC) (also called “Peripheral Blood Mononuclear Cells”) from whole blood. The isolated WBC can then be used to determine Sirt1 gene haplotype as described herein, measure citrate synthase (CS, EC 4.1.3.7) activity or mitochondrial DNA (mtDNA) content. These latter two parameters represent markers of mitochondrial content in WBC. Changes in WBC CS activity or mtDNA over time in a given individual reflect changes in WBC mitochondrial oxidative capacity. Depending on the treatment (e.g. activation of mitochondriogenesis through a factor that is expressed in all tissues), changes in WBC mitochondrial oxidative capacity can reflect changes in mitochondrial oxidative capacity in other tissues (e.g. skeletal muscle, white adipose tissue).

This procedure is based on approximately 6 ml of whole blood (Vacutainer format). This is the content of a standard tube (Becton Dickinson Vacutainer™ CPT™ Cell Preparation Tubes with Sodium Heparin, cat.#362753). Mix the blood before centrifugation by 10 times gently inverting the tube up and down. Centrifuge the CPT-tubes 20 minutes at 1700 RCF (3100 RPM) at room temperature (18-25° C.) with the brake off. Open the CPT tube and remove the plasma (4 ml) without disturbing the cell phase. Store the plasma if necessary. Remove the cell phase (ca. 2 ml, containing WBC, platelets and some plasma) with a plastic Pasteur (transfer) pipette and transfer this phase to a 15 ml conical Falcon-tube. Add phosphate buffered saline (PBS) to the cells to bring the volume up to 13 ml. Mix carefully by inverting the tube. Centrifuge the 15 ml conical tube at 300 RCF (1200 RPM) for 15 minutes at room temperature (18-25° C., no brake). Aspirate the supernatant (PBS, platelets and some plasma) without disturbing the cell pellet, and resuspend the cell pellet (WBC) in the remaining PBS (approximately 200 μl). Add PBS to the remaining cell suspension to bring the volume up to 13 ml, mix carefully by inverting the tube. Centrifuge at room temperature at 300 RCF (1200 RPM) for 15 minutes at room temperature (18-25° C., no brake). Aspirate the supernatant without disturbing the cell pellet, and resuspend the cell pellet in the remaining PBS (approximately 200 μl). Add PBS to the remaining cell suspension to bring the volume up to 10 ml, mix carefully by inverting the tube. Centrifuge at room temperature at 300 RCF (1200 RPM) for 15 minutes at room temperature (18-25° C., no brake). Aspirate the supernatant without disturbing the cell pellet. From this point keep the cells on ice.

Add 1 ml Freeze Medium without FBS( RPMI Medium 1640 with L-Glutamine; DMSO (dimethyl sulfoxide), 10% (vol:vol) final) to the remaining cell pellet and resuspend the cells gently. For some uses where plasma proteins do not interfere with the assay, e.g. for mtDNA quantification (but NOT for CS activity measurement), the WBC pellet can be resuspended and frozen in Freeze Medium with FBS (RPMI Medium 1640 with L-Glutamine; DMSO (dimethyl sulfoxide), 10% (vol:vol) final; FBS (Fetal Bovine Serum), heat inactivated 30 minutes at 56° C., 20% (vol:vol) final. Plasma proteins help maintain cell integrity when frozen. Once the Freeze Medium is added the cells must remain on wet ice for the remainder of the process and should be frozen as soon as possible. Transfer the cell suspension into cryovials (2 aliquots of 0.5 ml per sample). Freeze the cryovials by placing them into a −80° C. freezer. Keep the WBC samples at −80° C. until use. Six mililiters of blood gives around 10 million WBC, containing around 4 μg total RNA, 40 μg total cell proteins and 0.15 ng SIRT1 protein.

600-800 million WBC corresponding to ˜0.26 nM of SIRT1 in 20 μL of final lysate are used for a standard experiment to measure the activity of SIRT1 with five time points in triplicate for two given sets of experiments. The amount of SIRT1 in each preparation is determined initially by Western-Blot analysis using different amounts of WBC with a given SIRT1 standard (purified SIRT1, bacterially expressed).

The WBC are thawed and collected in a single 15 mL falcon tube at 4 degrees Celsius. The assay buffer consists of 10× reaction buffer, 5 mM DTT and 0.05% BSA. The reaction buffer is prepared as a 10× stock and consists of 500 mM Tris HCl pH 8.0, 1370 mM NaCl, 27 mM KCl, and 10 mM MgCl₂. The buffer is stored at room temperature. Prior to use the final assay buffer is chilled at 4 degrees Celsius. 700 μL of assay buffer is added to the collected WBC and gently mixed. Cells are sonicated on ice for 2 minutes with intervals (15 seconds sonication, 30 seconds pause) at a power output level of 1.5 with a small sonicator probe (Virsonic sonicator). The sonicated cells are centrifuged for 5 minutes at 3000 rpm and the supernatant (referred as “lysate”) is removed for further use in the activity assay.

Alternatively, lysates can be prepared from tissue, such as liver, fat or muscle. Typically, two to six pieces of one liver (approx, 500 mg) or two pieces of muscle (approx, 180 mg) corresponding to ˜0.26 nM of SIRT1 in 20 μL of final lysate are used for a standard experiment to measure the activity of SIRT1 with five time points in triplicate for two given sets of experiment. The amount of SIRT1 in each preparation is again determined initially by Western-Blot analysis using different amounts of mouse liver lysates or muscle lysates with a given SIRT1 standard (purified SIRT1, bacterially expressed). 700 μL of assay buffer are added to the collected tissues and gently mixed. Then these tissues are homogenized on ice using a Polytron for 20 seconds at maximum speed. (Omni International GLH). The homogenized tissues are centrifuged for 5 minutes at 13.000 rpm and the supernatant (referred as “lysate”) is removed for further use in the activity assay.

Finally, lysates can also be prepared from cell lines, such as those derived from liver, muscle, fat etc. The following describes preparation of lysates from myoblast C2C12 cell line. Myoblast cells are grown to 80% confluence and harvested with TrypLE (Invitrogen), then washed twice with PBS buffer (Invitrogen) and stored at −80 degree Celsius prior to use. A C2C12 myoblast cell pellet ˜100 to 200 mg corresponding to ˜0.26 nM of SIRT1 in 20 μL of final lysate is used for a standard experiment to measure the activity of SIRT1 with five time points in triplicate for two given sets of experiment. The amount of SIRT1 in each preparation is determined initially by Western-Blot analysis using different amounts of cells with a given SIRT1 standard (purified SIRT1, bacterially expressed). 700 μL of assay buffer are added to the collected myoblast cells and gently mixed. Then these cells are sonicated on ice for 2 minutes with intervals (15 seconds sonication, 30 seconds pause) at a power output level of 1.5 with a small sonicator probe (Virsonic sonicator). The sonicated cells are centrifuged for 5 minutes at 3000 rpm and the supernatant (referred as “lysate”) is removed for further use in the activity assay. 20 uL of lysate are taken typically for one well of a 96 well plate with a final total reaction volume of 100 uL.

20 uL of lysate are taken typically for one well of a 96 well plate with a final total reaction volume of 100 uL. 1 uL of DMSO is added to each of the wells to give a final concentration of 1%. 29 uL of assay buffer are added to an initial volume of 50 uL. Stop buffer (10% trichloroacetic acid and 500 mM Nicotinamide) is added to the wells designated to zero time points. The activity assay is started by adding 50 uL of substrate buffer to each well. The substrate buffer consists of 20 μM Tamra peptide Ac-Glu-Glu-Lys(Biotin)-Gly-Gln-Ser-Thr-Ser-Ser-His-Ser-Lys(Ac)-Nle-Ser-Thr-Glu-Gly-Lys(5TMR)-Glu-Glu-NH2 (SEQ ID NO:2) wherein K(Ac) is an acetylated lysine residue and Nle is a norleucine. The peptide is labeled with the fluorophore 5TMR (excitation 540 nm/emission 580 nm) at the C-terminus for use in the FP assay described above. The peptide substrate is prepared as a 1 mM stock in distilled water and stored in aliquots at −20° C.), 5 mM DTT, 0.05% BSA, 4 mM NAD⁺ and 10× reaction buffer. The reaction is performed at RT. For each time point the reaction will be stopped with stop buffer. After the final time point is collected the plates are sealed and analyzed by mass spectrometry.

As controls, specific SIRT1 and HDAC inhibitors are also included in the assay. Lysate volumes are adjusted accordingly to the amount needed for this inhibition assay. The following inhibitors are used with their respective final concentrations: 6-chloro-2,3,4,9-tetrahydro-1-H-carbazole-l-carboxamide (5 μM), TSA (1 μM) and nicotinamide (5 mM). 6-chloro-2,3,4,9-tetrahydro-1-H-carbazole-1-carboxamide and TSA are prepared in DMSO. Nicotinamide preparations are made in water. The final concentration of DMSO in each well is 1%. 1 L of DMSO is added to wells containing Nicotinamide as inhibitor. The reactions are run in duplicate over a time period of 90 to 120 minutes with at least 5 time points taken.

Assay plates are transferred to BioTrove, Inc. (Woburn, Mass.) on dry ice for mass spectrometry analysis. Thawed reactions are analyzed using an Agilent 1100 HPLC with a microplate autosampler linked in series with a Sciex API-4000 mass spectrometer. Proprietary equipment (developed by BioTrove, Inc.) has been incorporated into this LC-MS system to allow for rapid sampling and rapid sample clean-up (4-5 sec per well). Both substrate and product are tracked in the MS and the area of the MS curve for both product and substrate are reported back in arbitrary units.

Using Microsoft Excel, plot product on the x axis and reaction time on the y axis of a xy scatter plot. The reaction is run at saturating substrate conditions with deliver a maximal turnover of substrate to product over a fixed time period, necessary for the detection of the activity of SIRT1. The final readout will be a number/slope describing product accumulation/time/ng of enzyme. Inhibition of the enzymatic activity of SIRT1 results in low product yields that enable the differentiation between HDAC's and SIRT1.

### Example 5

### In Vivo Effects of Sirtuin Activation in a Diet Induced Obesity (DIO) Mouse Model

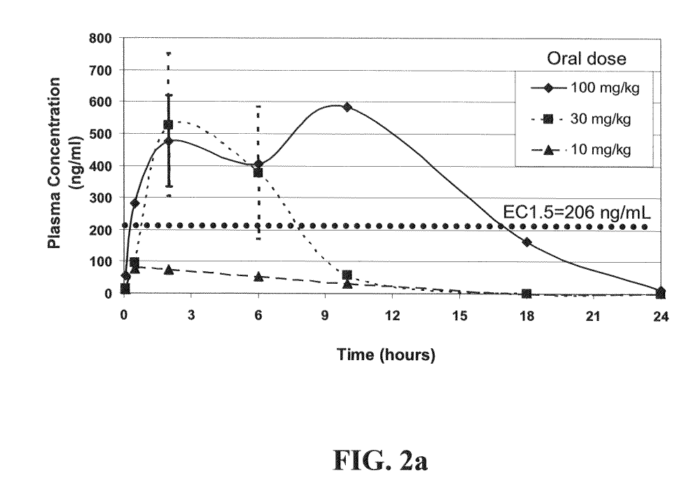

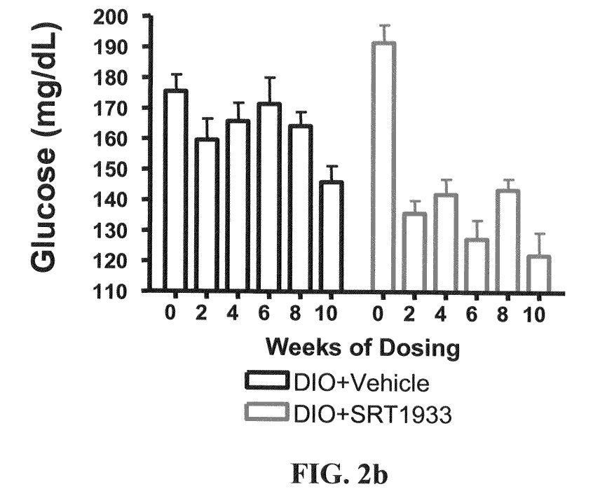

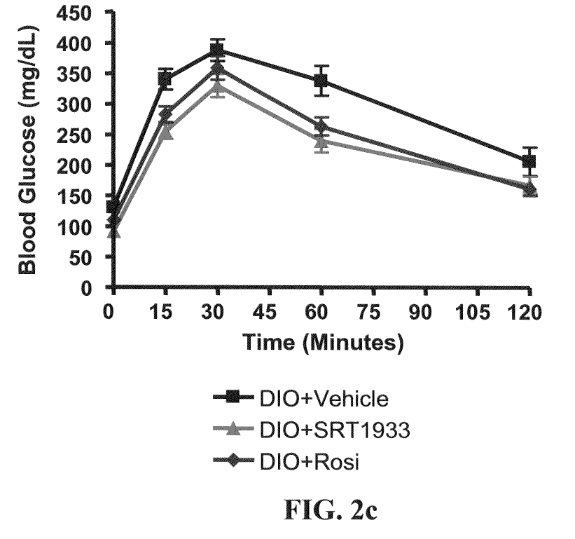

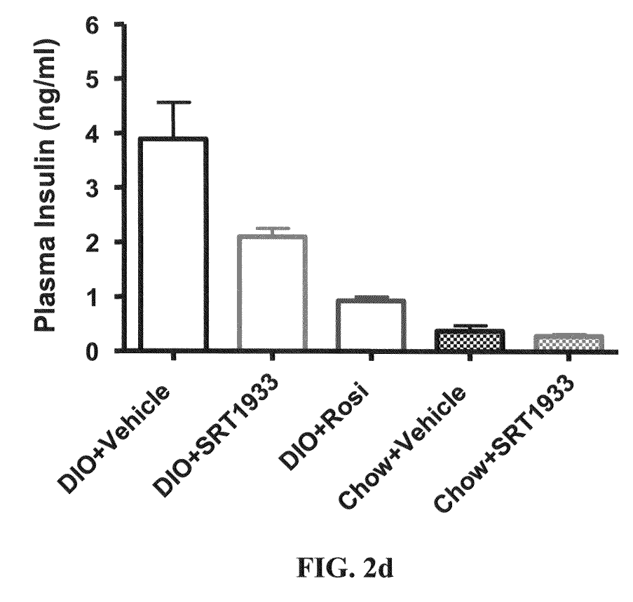

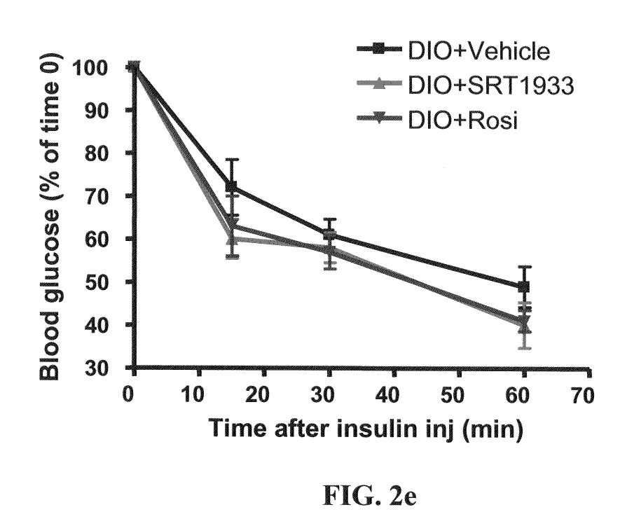

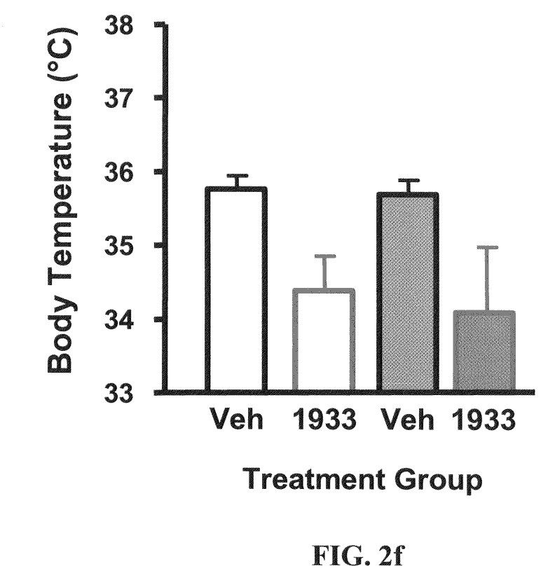

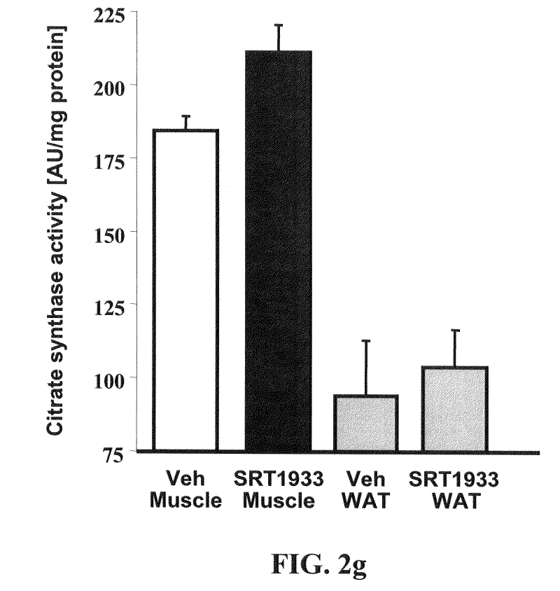

The following example describes the in vivo effects of a novel SIRT1 activator. The compound was administered via oral gavage at the doses indicated in FIG. 2a in C57BLU6 male mice, 18-22 grams (Wilmington, Mass. Charles River Labs), 3 mice per group and blood plasma was collected at 5, 30, 120 and 360 minute time points. The compound was administered in 2% HPMC+0.2% DOSS. Mice were sacrificed at proper time points using CO₂ overdose (place in CO₂ chamber 40 seconds before time point). Blood was collected in microtainer blood tubes with Lithium Heparin and plasma separator and frozen plasma was sent to Charles River Labs (CRL) for analysis.

To determine oral bioavalability, the compound was administered into the tail vein at 0.1, 0.3 and 1.0 mg/kg doses in C57BL/6 male mice, 18-22 grams (Wilmington, Mass. Charles River Labs), 3 mice per group and blood plasma was collected at 5, 30, 120 and 360 minute time points. Compounds were administered in 10% ethanol/ 40% Polyethylene glycol/50% H2O for IV studies. Mice were sacrificed at proper time points using CO₂ overdose (place in CO₂ chamber 40 seconds before time point). Blood was collected and analyzed as described above.

For the diet induced obesity model, six week old C57BU6 male mice (Charles River Labs) were fed a high fat diet (60% calories from fat; Research Diets) for approximately 6 weeks until their body weight reached ˜40 g. Test compounds were administered once daily via oral gavage at 100 mg/kg SRT1933 or 5 mg/kg rosiglitazone. The vehicle used was 2% HPMC −0.2% DOSS. Individual mouse body weights were measured twice weekly. Every 2 weeks throughout the study, mice from each group were bled via the tail vein for determination of blood glucose and blood plasma insulin. After 1, 3 and 5 weeks of dosing, a fasted blood glucose measure was taken and after 5 weeks of treatment an IPGTT was conducted on all mice from each of the groups. After 9 weeks of treatment, an ITT was conducted and a rectal body temperature was taken on all mice from each of the groups. Statistical analysis was completed using the JMP program (Version 6). Data were analyzed by a one way ANOVA with comparison to control using a Dunnett's Test. A p value <0.05 indicated a significant difference between groups.

Citrate synthase (CS) activity in skeletal muscle (gastrocnemius) and white adipose tissue (epididymal) was determined after 11 weeks of treatment using the method described by Srere (Citrate synthase, Methods Enzymol. 1969, 13: 3-5) and Moyes et al. (Moyes CD, Mathieu-Costello 0A, Tsuchiya N, Filbum C, and Hansford RG. Mitochondrial biogenesis during cellular differentiation. Am. J. Physiol. 1997, 272 (Cell Physiol. 41): C1345-C1351.

Citrate Synthase is an enzyme of the Krebs cycle (also called tricyclic acid, TCA, or citric acid) cycle whose maximal activity (capacity) reflects mitochondrial oxidative capacity of the sample (Holloszy et al. 1970, Williams et al. 1986, Hood et al. 1989). This is true at least with samples from aerobic organisms (e.g. mammals) that do not harbor a major defect in a particular mitochondrial oxidative phosphorylation (OXPHOS) subunit, as can be found in specific mitochondrial myopathies. Irrespective of this latter remark, changes in citrate synthase activity over time in a given individual reflect changes in tissue mitochondrial oxidative capacity. The five mice best representing the mean fasting blood glucose level of each group (DIO Vehicle and DIO SRT1933) were selected for this analysis. The tissues were lyzed in lx Extraction Buffer (20 mM N-2-hydroxyethylpiperazine-N′-2-2ethanesulfonic acid (HEPES), pH 7.2, 0.1% Triton X-100 and 1 mM EDTA with a polytron tissue homogenizer (OMNI International GLH) for 30 seconds at maximum speed (setting 6) on ice. A low-speed (13,000 rpm, microfuge) centrifugation step was then used to pellet big cell debris. The supernatant was used to assess citrate synthase activity. The samples are freeze-thawed 2 times to break the mitochondrial membrane and allow access to citrate synthase. Sample protein concentrations were determined according to the supplier's instructions, with Bio-Rad Protein Assay Dye Reagent Concentrate (cat#500-0006, Sigma) with Protein Standard I, bovine gamma globulin (cat#500-0005, Sigma) as standard. The citrate synthase activity assay was performed in a final total volume of 300 μl with 100 μg of protein per sample, measured in triplicate.

Citrate synthase catalyzes the reaction between acetyl coenzyme A (acetyl CoA) and oxaloacetic acid (OAA) to form citric acid. In this reaction, the hydrolysis of the thioester of acetyl CoA results in the formation of CoA with a thiol group (CoA-SH). The thiol reacts with 5,5′dithiobis-(2-nitrobenzoic acid) (DTNB) in the reaction mixture to form 5-thio-2-nitrobenzoic acid (TNB). CS activity was measured spectrdphotometrically by monitoring the absorbance at 412 nm of TNB (yellow product). The assay buffer contained 0.1 mM DNTB, 0.3 mM acetyl CoA, and 0.5 mM oxaloacetate in 50 mM tris (hydroxymethyl) aminomethane (Tris-HCl), pH 8.0. The reaction was started with addition of assay buffer containing DNTB to the tissue lysates, and the increase in absorbance at 412 nm was measured every 30 seconds over 15 min at 24° C. (software SoftMax Pro 4.8). The maximal enzyme activity (mU per min) is given by the slope of the formation of TNB (absorbance) over time in the linear part of the reaction, obtained between 100 seconds and 900 seconds (end of measurement). Each sample was assayed in triplicate and expressed as the mean Vmax in arbitrary units/mg protein.

### Results

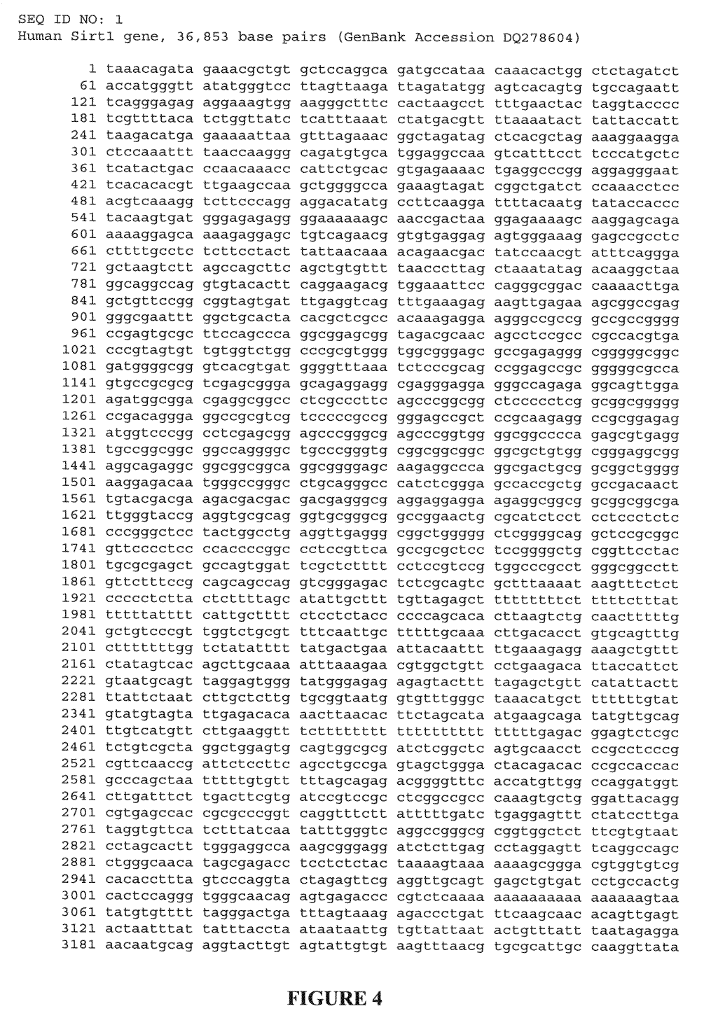

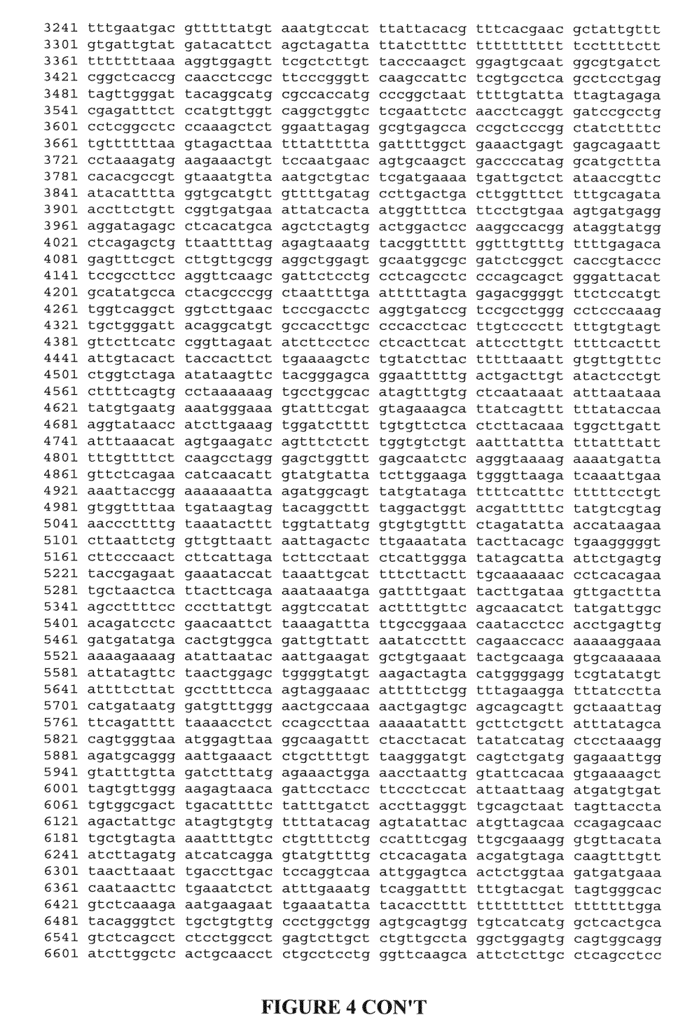

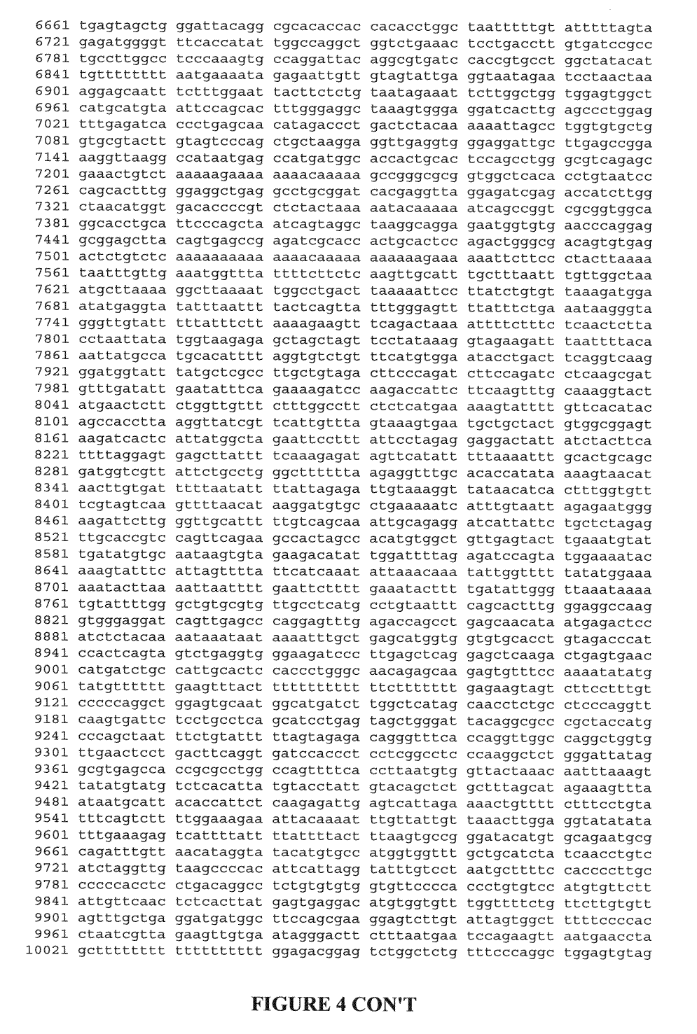

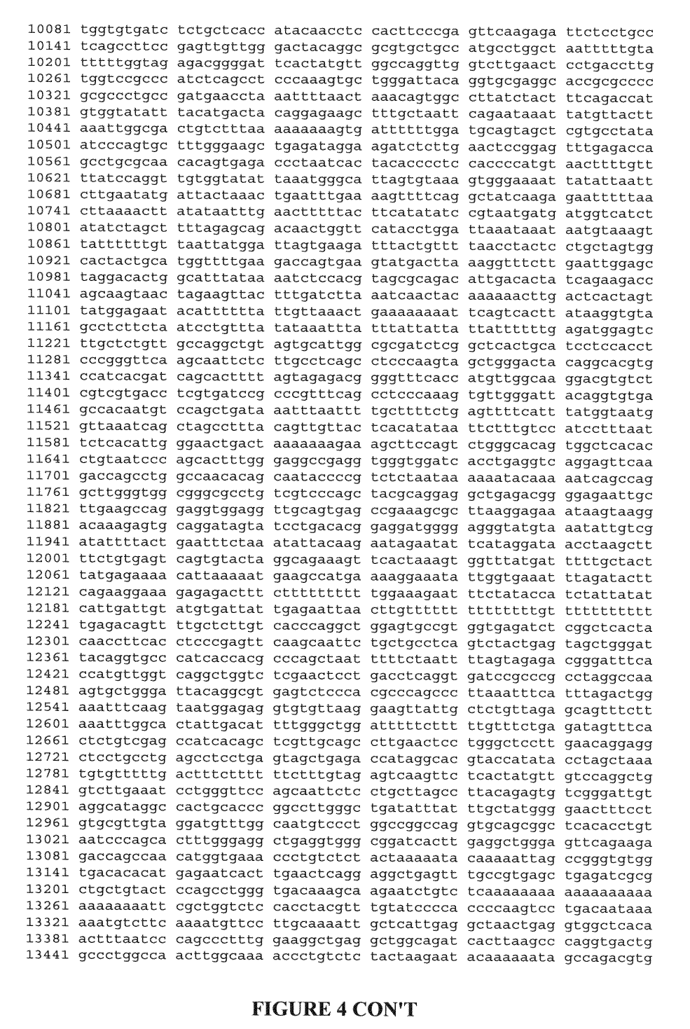

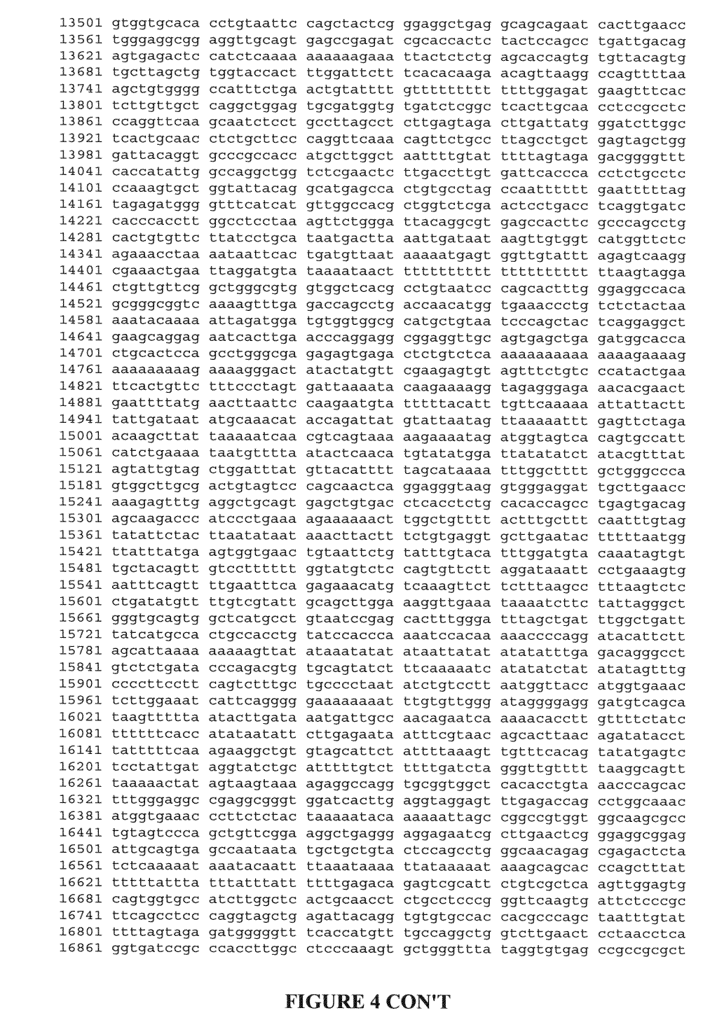

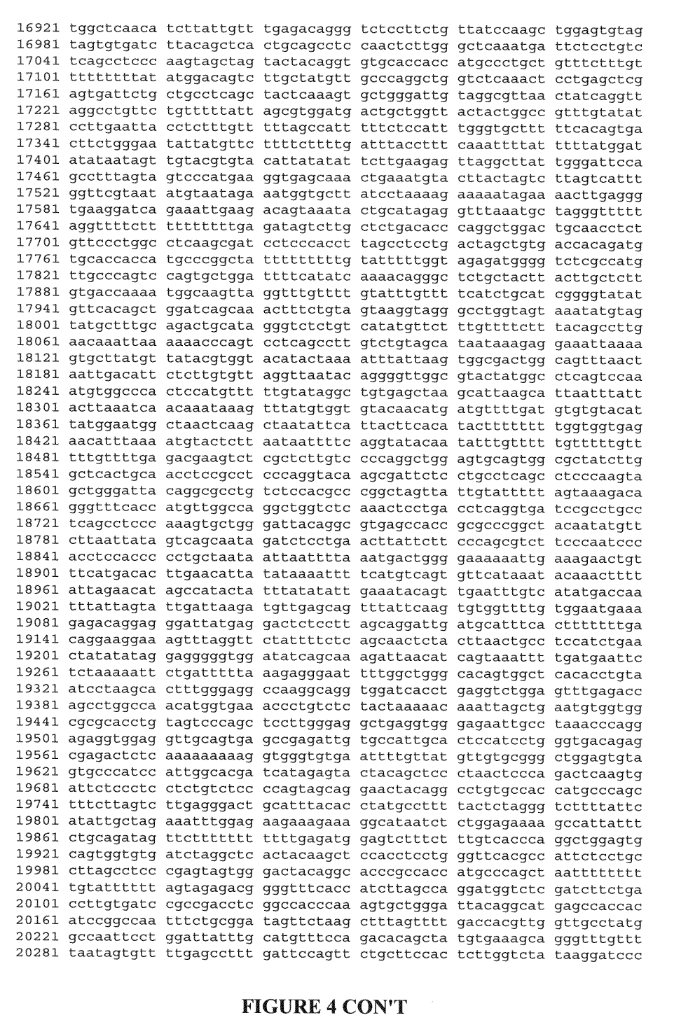

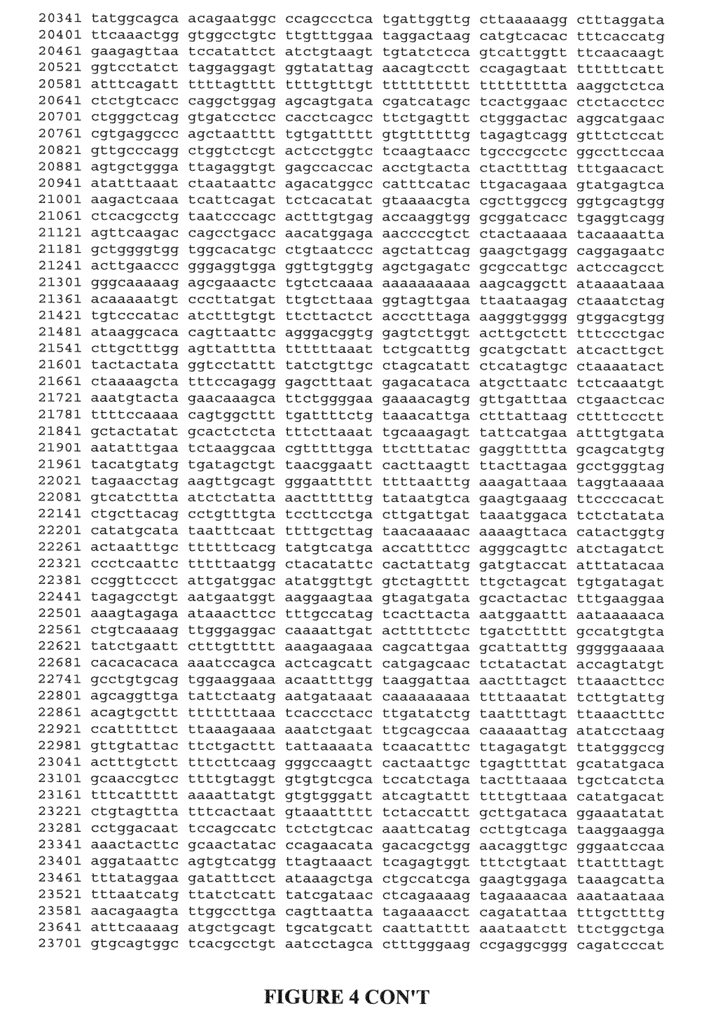

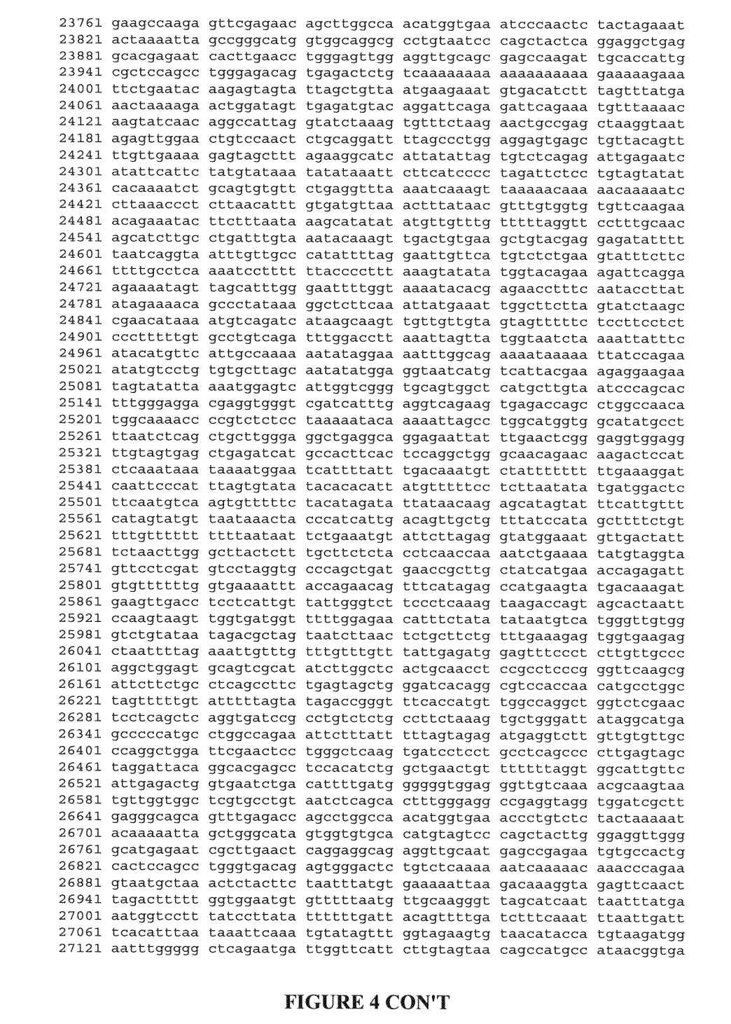

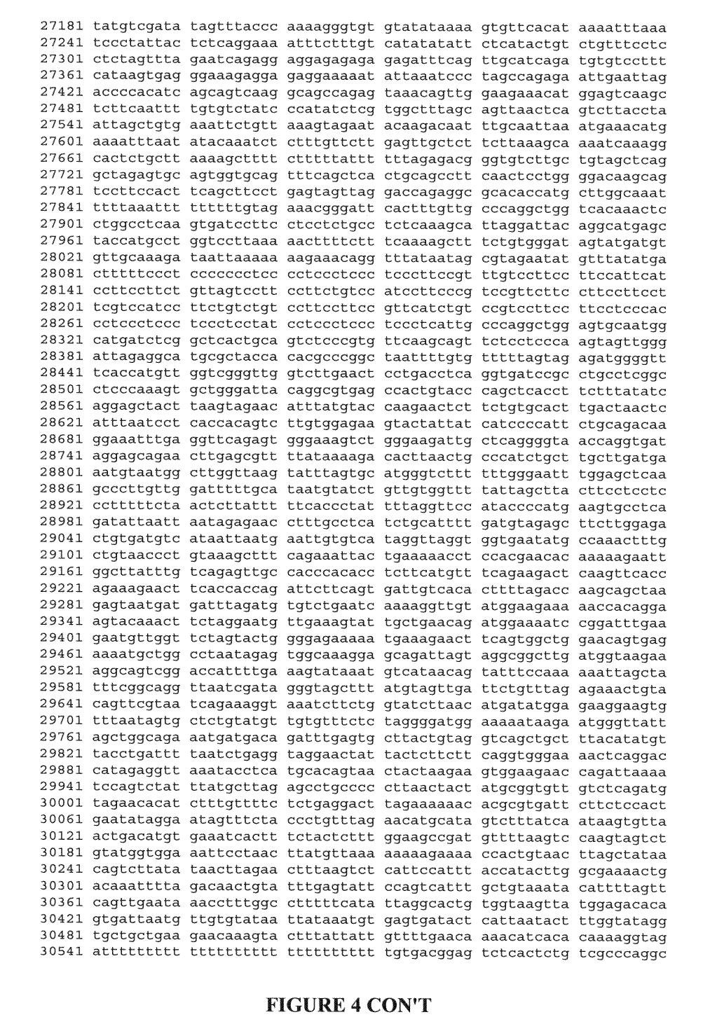

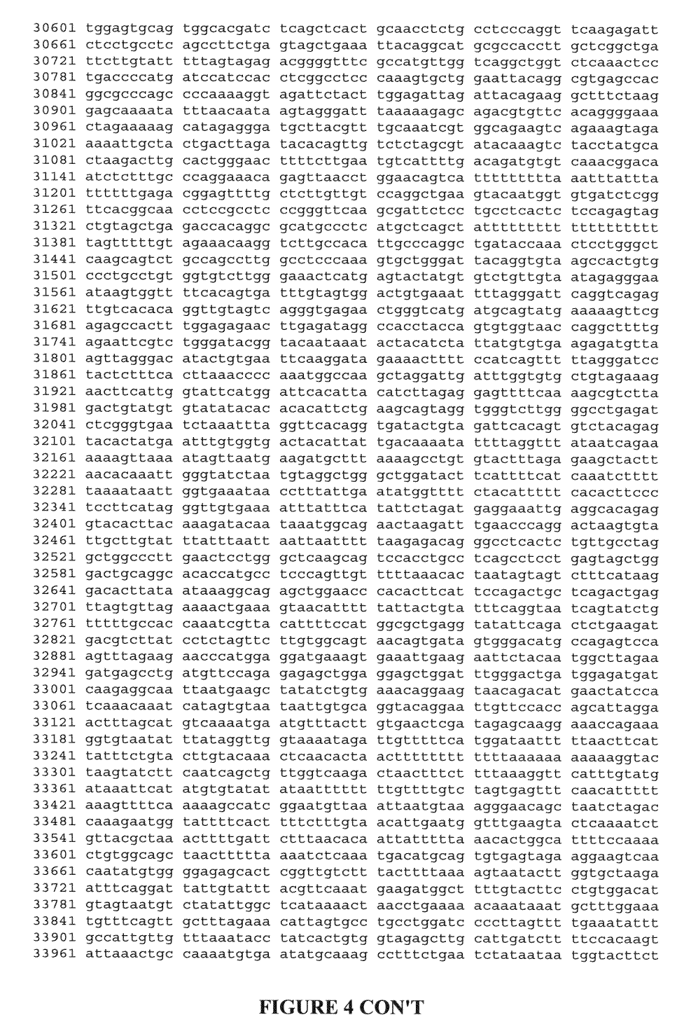

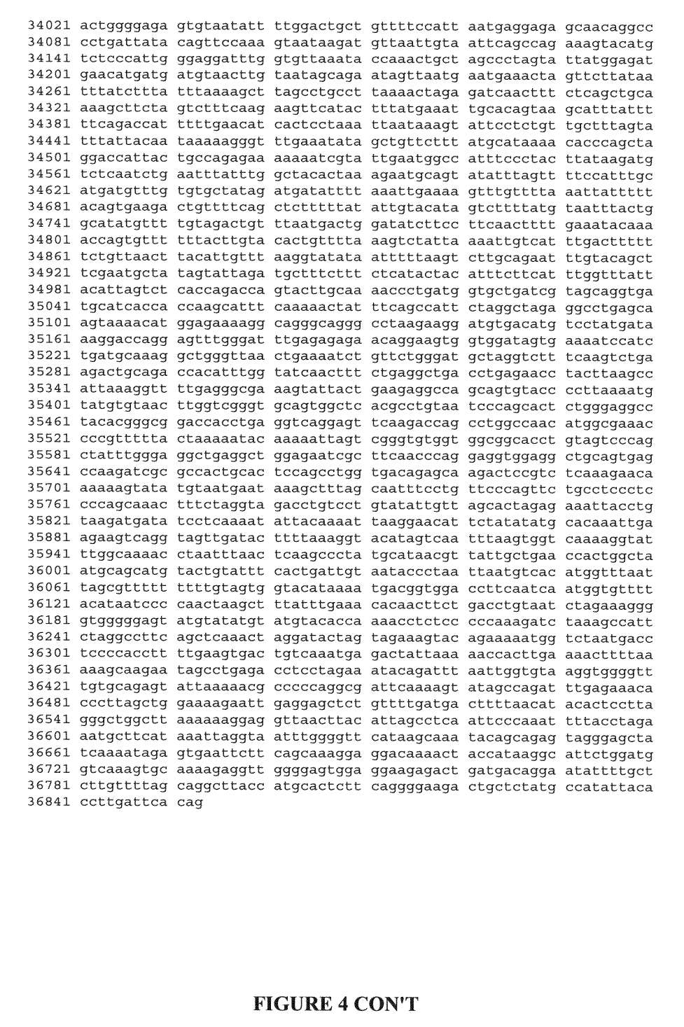

This example describes the potential utility of a novel SIRT1 activator to treat insulin resistance and diabetes utilizing a mouse model of mild diabetes and insulin resistance, the diet induced obese (DIO) mouse³⁴. SRT1933 was given once daily by oral gavage at a dose of 100 mg/kg which resulted in compound exposure above its EC_{1.5} for at least 16 hours (FIG. 2a). In the DIO mouse model, nonfasting blood glucose levels are elevated (150-200 mg/dL range) and administration of SRT1933 over a 10 week period normalized nonfasting glucose levels by week 2 and maintained these levels over the remainder of the study (FIG. 2b). In addition fasting blood glucose levels were normalized. The DIO mice treated with SRT1933 had improved glucose tolerance in an OGTT relative to the vehicle treated group and this improvement was similar to that produced by rosiglitazone, a PPARγ activator. This effect was assessed by measuring the glucose excursion for the DIO vehicle (AUC=603±32 mg.hr/dL), SRT1933 (AUC=462±25 mg.hr/dL) and rosiglitazone (AUC=496±20 mg.hr/dL) groups after 5 weeks of treatment. The DIO animals are also hyperinsulinemic (3.9±0.7 ng/mL) compared to chow fed controls (0.4±0.1 ng/mL) due to obesity induced insulin resistance. SRT1933 (2.1±0.1 ng/mL) significantly reduced the hyperinsulinemia (FIG. 2d) partially normalizing the elevated insulin levels and Rosiglitazone (0.9±0.1 ng/mL) had a similar effect in this model. SRT1933 did not have a significant effect on insulin levels in mice fed a normal chow diet over the course of the study (FIG. 2d). An improvement in insulin sensitivity relative to vehicle as assessed by an ITT was observed with both SRT1933 and rosiglitazone (FIG. 2e). One of the hallmarks of calorie restriction is a slight reduction in body temperature^{1,35}. Recently, Conti et al. showed increased lifespan in transgenic mice that exhibited a reduced core body temperature³⁶. Like CR, a ˜1° C. lowering of core body temperature in the SRT1933 treatment groups in both the DIO mice and normal chow fed mice was also observed (FIG. 2f). Interestingly, as also observed in calorie restriction, mitochondrial capacity is elevated following treatment with SRT1933 as measured by an increase in citrate synthase activity of 15% in the gastrocnemius muscle (FIG. 4g)^{30,35}. Taken together the data from the DIO study suggest that SIRT1 activation mimics several of the effects observed following calorie restriction including improved insulin sensitivity, normalized glucose and insulin levels, elevated mitochondrial function, and lower core body temperature. The effects observed also support the therapeutic potential of SIRT1 activators for the treatment of Type 2 Diabetes. Like CR, SRT1933 reduces blood glucose and insulin, improves insulin sensitivity, induces mitochondrial biogenesis and reduces core body temperature.

### Example 6

### Evaluation of SIRT1 Polymorphisms

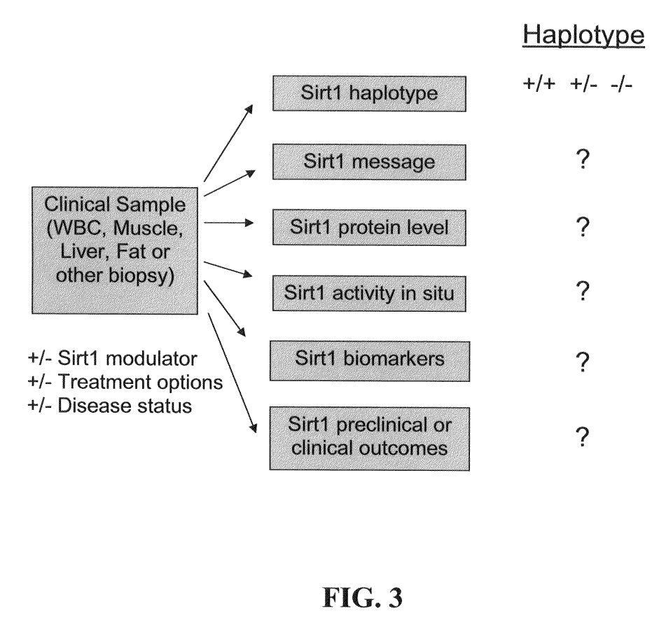

The following example describes a protocol (depicted in FIG. 3) for establishing the interrelationship between the Sirt1 genetic polymorphisms described herein and either environmental or physiological status or manipulation (diet, exercise, age, disease progression, etc.) or following pharmacological intervention (including Sirt1 modulators or other therapeutic interventions) including studies designed to study dose responsiveness and escalation, vehicle or placebo control versus treatment groups, dosing regimen, drug combination and synergy, etc. Cells, tissue or clinical samples (herein referred to as sample) can be from heart, kidney, brain, liver, bone marrow, colon, stomach, upper and lower intestine, breast, prostate, thyroid, gall bladder, lung, adrenals, muscle, fat, nerve fibers, pancreas, skin, eye, etc. Preferred samples include blood, white blood cells, liver, muscle, fat and other tissues that are the target of Sirt1 pharmacological intervention. The cells or tissues can be pretreated with Sirt1 modulators or other pharmacological agents either in vivo or following isolation.

The genetic analysis of haplotypes, SNPs or alleles of the Sirt1 gene as described herein could be done on the samples collected above. It is of course understood that in general the genetic analysis need not be done on the same sample used for subsequent biochemical analysis. Any sample, tissue or biopsy obtained from the given patient should be sufficient to determine the genetic haplotype of the Sirt1 gene as well as genetic analysis of any other gene. As depicted in FIG. 3, the haplotype is schematically represented as +/+, ± or −/−hfor the Sirt1 allele of interest.

The sample can then be subjected to a number of other biochemical and/or biological studies. These include quantitative measurement of mRNA or protein by methods known in the art and described herein. Of particular interest would be the measurement of Sirt1 mRNA or protein. Other gene products of interest include the PGC-1α mRNA and protein and genes related to OXPHOS (Lin et al., 2002, J. Biol. Chem. 277, 1645-1648); the estrogen related receptor alpha (ERRα) and nuclear respiratory factorI (NRF-1) mRNA and protein (Mootha et al., 2004, Proc Natl Acad Sci U S A 101, 6570-6575; Patti et al., 2003, Proc Natl Acad Sci U S A 100, 8466-8471); Mitochondrial transcription factor A (Tfam), a nuclear encoded mitochondrial transcription factor that is indispensable for the expression of key mitochondrial-encoded genes (Larsson et al., 1998, Nat. Genet. 18, 231-236) and a target of NRF-1; an array of additional downstream targets of PGC-1α (Lin et al., 2005, Cell Metab 1, 361-370), including genes involved in fatty acid oxidation (medium chain acyl-CoA dehydrogenase, MCAD), uncoupling and protection against ROS (uncoupling protein 3, UCP-3), and fiber type markers (myoglobin and troponin 1).

In addition to the measurement of protein and mRNA of specific gene products as described above, the measurement of endogenous activity can be m easured. This includes the determination of endogenous sirtuin activity in various clinical samples with or without physiological manipulation or pharmacological intervention. Of particular interest is the measurement of endogenous sirtuin activity as described in Example 4 above. Other activities can also be measured, including citrate synthase as described above in Example 5, ATP synthase, or where possible, any of the other gene products described above in this example. Finally, other mRNA, protein and/or activities that could be measured include those associated with mitochondrial biogenesis and disease progression or pathogenesis as described elsewhere in this specification. This includes ATP levels, mitochondrial number and size, mitochondrial DNA, oxidative phosphorylation markers, reactive oxygen species, etc. Specific mRNA and protein levels can be measured for the following: SIRT1, PGC-1alpha, mtTFA (TFAM), UQCRB, Citrate synthase, Foxo1, PPARgamma2, PPARdelta, LXRalpha, ABCA1, aP2, Fatty acid synthase, Adiponectin (13 genes), PGC-1beta, PPARgamma1, MIF (macrophage migration inhibition factor), MMP-9, TNFalpha, IL- 1alpha, IL-1beta, IL-12alpha, IL-18, IL-18BP (IL-18 binding protein), COX2 (cyclooxygenase-2), Lipoprotein lipase (LPL), resistin, IL-8, IL8Receptor, MCP1, MCP1-Receptor, MIP1alpha, MIP2alpha, MIP2beta, MMP-10, MIP1, VCAM, IL-6, TLR4, TLR2, ANGP1.

The correlation can then be made between Sirt1 haplotype (in combination with genetic analysis of other genes of interest) with mRNA, protein and activity of Sirt1 or any of the other gene products described above. This analysis can then be extended to preclinical or clinical outcome analysis, especially when looking at pharmacological intervention, herein referred to as pharmacogenetics or pharmacogeneomics. This includes prevention and/or intervention in diseases or disorders including reversal of disease or slowing the rate of progression, attenuation of disease markers, or holding of disease status or limiting disease progression. Specific diseases or disorders include those related to aging or stress, diabetes, obesity, neurodegenerative diseases, diseases or disorders associated with mitochondrial dysfunction, chemotherapeutic induced neuropathy, neuropathy associated with an ischemic event, ocular diseases and/or disorders, cardiovascular disease, blood clotting disorders, inflammation, oncology, asthma, COPD, rheumatoid arthritis, irritable bowel syndrome, psoriasis, and/or flushing, etc. Efficacy readouts for metabolic, diabetes or obesity related indications include glycosylated HbA 1C, fasting or post prandial glucose levels, glucose tolerance or insulin sensitivity, plasma insulin levels, etc. for metabolic indications. Other readouts include core body temperature, exercise endurance, energy expenditure, reactive oxygen species (ROS) levels, and other measurements of mitochondrial function or biogenesis as described herein. Neurological indications and clinical readouts include those known in the art and include such diseases as, for example, AD (Alzheimer's Disease), multiple sclerosis (MS), ADPD (Alzheimer's Disease and Parkinsons's Disease), HD (Huntington's Disease), PD (Parkinson's Disease), Friedreich's ataxia and other ataxias, amyotrophic lateral sclerosis (ALS) and other motor neuron diseases, optic neuritis, glaucoma and other related eye diseases, MELAS and LHON. Based on haplotype analysis, biochemical and clinical parameters, clinical intervention can be assessed based on dose responsiveness and escalation, vehicle or placebo control versus treatment groups, dosing regimen, drug combination and synergy, etc.

### REFERENCES

1. Heilbronn, L. K. & Ravussin, E. Calorie restriction and aging: review of the literature and implications for studies in humans. Am J Clin Nutr 78, 361-9 (2003).
2. Roth, G. S., Ingram, D. K. & Lane, M. A. Caloric restriction in primates and relevance to humans. Ann N Y Acad Sci 928, 305-15 (2001).
3. Weindruch, R. et al. Caloric restriction mimetics: metabolic interventions. J Gerontol A Biol Sci Med Sci 56 Spec No 1, 20-33 (2001).
4. Ingram, D. K. et al. Calorie restriction mimetics: an emerging research field. Aging Cell 5, 97-108 (2006).
5. Bordone, L. & Guarente, L. Calorie restriction, SIRT1 and metabolism: understanding longevity. Nat Rev Mol Cell Biol 6, 298-305 (2005).
6. Sinclair, D. A. & Guarente, L. Extrachromosomal rDNA circles—a cause of aging in yeast. Cell 91, 1033-42 (1997).
7. Kaeberlein, M., McVey, M. & Guarente, L. The SIR2/3/4 complex and SIR² alone promote longevity in Saccharomyces cerevisiae by two different mechanisms. Genes Dev 13, 2570-80 (1999).
8. Rogina, B. & Helfand, S. L. Sir2 mediates longevity in the fly through a pathway related to calorie restriction. Proc Natl Acad Sci U S A 101, 15998-6003 (2004).
9. Anderson, R. M., Bitterman, K. J., Wood, J. G., Medvedik, 0. & Sinclair, D. A. Nicotinamide and PNC1 govern lifespan extension by calorie restriction in Saccharomyces cerevisiae. Nature 423, 181-5 (2003).
10. Lin, S. J., Defossez, P. A. & Guarente, L. Requirement of NAD and SIR2 for life-span extension by calorie restriction in Saccharomyces cerevisiae. Science 289, 2126-8 (2000).
11. Cohen, H. Y. et al. Calorie restriction promotes mammalian cell survival by inducing the SIRT1 deacetylase. Science 305, 390-2 (2004).
12. Heilbronn, L. K. et al. Glucose tolerance and skeletal muscle gene expression in response to alternate day fasting. Obes Res 13, 574-81 (2005).
13. Howitz, K. T. et al. Small molecule activators of sirtuins extend Saccharomyces cerevisiae lifespan. Nature 425, 191-6 (2003).
14. Jarolim, S. et al. A novel assay for replicative lifespan in Saccharomyces cerevisiae. FEMS yeast research 5, 169-77 (2004).
15. Wood, J. G. et al. Sirtuin activators mimic caloric restriction and delay ageing in metazoans. Nature (London, United Kingdom) 430, 686-689 (2004).
16. Baur, J. A. et al. Resveratrol improves health and survival of mice on a high-calorie diet. Nature (2006).
17. Frye, R. A. Characterization of five human cDNAs with homology to the yeast SIR2 gene: Sir2-like proteins (sirtuins) metabolize NAD and may have protein ADP-ribosyltransferase activity. Biochem Biophys Res Commun 260, 273-9 (1999).
18. Frye, R. A. Phylogenetic classification of prokaryotic and eukaryotic Sir2-like proteins. Biochem Biophys Res Commun 273, 793-8 (2000).
19. Imai, S., Armstrong, C. M., Kaeberlein, M. & Guarente, L. Transcriptional silencing and longevity protein Sir2 is an NAD-dependent histone deacetylase. Nature 403, 795-800 (2000).
20. Bouras, T. et al. SIRT1 deacetylation and repression of p300 involves lysine residues 1020/1024 within the cell cycle regulatory domain 1. J Biol Chem 280, 10264-76 (2005).
21. Brunet, A. et al. Stress-dependent regulation of FOXO transcription factors by the SIRT1 deacetylase. Science 303, 2011-5 (2004).
22. Luo, J. et al. Negative control of p53 by Sir2alpha promotes cell survival under stress. Cell 107, 137-48 (2001).
23. Motta, M. C. et al. Mammalian SIRT1 represses forkhead transcription factors. Cell 116, 551-63 (2004).
24. Nemoto, S., Fergusson, M. M. & Finkel, T. SIRT1 functionally interacts with the metabolic regulator and transcriptional coactivator PGC-1 {alpha}. J Biol Chem 280, 16456-60 (2005).
25. Picard, F. et al. Sirt1 promotes fat mobilization in white adipocytes by repressing PPAR-gamma. Nature 429, 771-6 (2004).
26. Rodgers, J. T. et al. Nutrient control of glucose homeostasis through a complex of PGC-1alpha and SIRT1. Nature 434, 113-8 (2005).
27. van der Horst, A. et al. FOXO4 is acetylated upon peroxide stress and deacetylated by the longevity protein hSir2(SIRT1). J Biol Chem 279, 28873-9 (2004).
28. Vaziri, H. et al. hSIR2(SIRT1) functions as an NAD-dependent p53 deacetylase. Cell 107, 149-59 (2001).
29. Yeung, F. et al. Modulation of NF-kappaB-dependent transcription and cell survival by the SIRT1 deacetylase. Embo J 23, 2369-80 (2004).
30. Nisoli, E. et al. Calorie restriction promotes mitochondrial biogenesis by inducing the expression of eNOS. Science 310, 314-7 (2005).
34. Van Heek, M. V. et al. Diet-induced obese mice develop peripheral, but not central, resistance to leptin. Journal of Clinical Investigation 99, 385-390 (1997).
35. Heilbronn, L. K. et al. Effect of 6-month calorie restriction on biomarkers of longevity, metabolic adaptation, and oxidative stress in overweight individuals: a randomized controlled trial. Jama 295, 1539-48 (2006).
36. Conti, B. et al. Transgenic mice with a reduced core body temperature have an increased life span. Science 314, 825-8 (2006).

### Equivalents

The present invention provides among other things predictive and diagnostic methods using polymorphic variants of Sirt1. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

1. A method for identifying a subject that would be responsive to treatment with a sirtuin modulating compound, comprising determining the presence or absence of at least one polymorphic variant in a biological sample from said subject, wherein the polymorphic variant is in a nucleic acid sequence that encodes a sirtuin protein or controls expression of a sirtuin gene, and wherein the presence of the at least one polymorphic variant is indicative of a subject that would be responsive to treatment with a sirtuin modulating compound.

2. The method of claim 1, wherein the polymorphic variant is associated with a lower level of sirtuin expression, activity or expression and activity.

3. The method of claim 1, wherein the sirtuin modulating compound is a sirtuin activating compound.

4. The method of claim 1, wherein the polymorphic variant is in a coding region of a sirtuin sequence.

5. The method of claim 1, wherein the polymorphic variant is in a non-coding region of a sirtuin sequence.

6. The method of claim 5, wherein the polymorphic variant is in a promoter region.

7. The method of claim 5, wherein the polymorphic variant is in an intronic region.

8. The method of claim 1, wherein the polymorphic variant is in human SIRT1.

9. The method of claim 8, wherein the human SIRT1 comprises the nucleic acid sequence set forth in SEQ ID NO: 1 or a portion thereof.

10. The method of claim 1, wherein the polymorphic variant is selected from the group consisting of: an A variant of single nucleotide polymorphism (SNP) rs3740051, an A variant of SNP rs2236319, or a T variant of SNP rs2273773.

11. The method of claim 1, wherein the biological sample is a blood sample, serum sample, or tissue sample.

12. The method of claim 1, wherein the presence or absence of the at least one polymorphic variant is determined using a polymerase chain reaction (PCR), restriction enzyme cleavage pattern, a nucleic acid probe that hybridizes to the nucleic acid sequence, or nucleic acid sequencing.

13. The method of claim 1, wherein the patient is suffering from a metabolic disease or disorder.

14. The method of claim 1, wherein the patient is suffering from a neurodegenerative disease or disorder.

15. The method of claim 1, further comprising administering a sirtuin modulating compound to said subject.

16. A method for identifying a subject that would benefit from treatment with a sirtuin modulating compound, comprising determining the presence or absence of at least one polymorphic variant in a biological sample from said subject, wherein the polymorphic variant is in a nucleic acid sequence that encodes a sirtuin protein or controls expression of a sirtuin gene, and wherein the presence of the at least one polymorphic variant is indicative of a subject that would benefit from treatment with a sirtuin modulating compound.

17-30. (canceled)

31. A method for evaluating a subject's risk of developing a sirtuin mediated disease or disorder, comprising determining the presence or absence of at least one polymorphic variant in a biological sample from said subject, wherein the polymorphic variant is in a nucleic acid sequence that encodes a sirtuin protein or controls expression of a sirtuin gene, and wherein the presence of the at least one polymorphic variant is indicative of a subject at risk for developing a sirtuin mediated disease or disorder.

32-45. (canceled)

46. A method for evaluating a sirtuin modulating compound, comprising:
a) administering a sirtuin modulating compound to a patient population;
b) determining the presence or absence of one or more polymorphic variants of a sirtuin sequence in a biological sample from the patients in said population before or after administering said sirtuin modulating compound to said patient population;
c) evaluating the efficacy of the sirtuin modulating compound in said patient population; and
d) correlating the efficacy of the sirtuin modulating compound with the presence or absence of the one or more polymorphic variants of the sirtuin sequence, thereby evaluating the sirtuin modulator.

47-65. (canceled)

66. A method for evaluating a sirtuin modulating compound, comprising:
a) administering a sirtuin modulating compound to a patient population for which the presence or absence of one or more polymorphic variants of a sirtuin sequence has been determined;
b) evaluating the efficacy of the sirtuin modulating compound in said patient population; and
c) correlating the efficacy of the sirtuin modulating compound with the presence or absence of the one or more polymorphic variants of the sirtuin sequence, thereby evaluating the sirtuin modulator.

67. A method for establishing the predictive value of a polymorphic variant of a sirtuin sequence, comprising:
a) determining the presence or absence of one or more polymorphic variants of a sirtuin sequence in a biological sample from patients in a patient population;
b) assaying one or more physiological or metabolic parameters in the patients of said patient population;
c) correlating the present or absence of the one or more polymorphic variants with the one or more physiological or metabolic parameters in said patient population, wherein a correlation is indicative of the predictive value of the polymorphic variant.

68-76. (canceled)

77. A method for treating a sirtuin mediated disease or disorder in a subject, comprising:
a) determining the presence or absence of one or more polymorphic variants in a sirtuin sequence in a biological sample from said subject, thereby producing a polymorphic variant profile for said subject;
b) analyzing the polymorphic variant profile to determine a course of treatment, dosage regimen, or course of treatment and dosage regimen for said subject; and
c) administering a sirtuin modulating compound to said subject according to the determined course of treatment, dosage regimen, or course of treatment and dosage regimen, thereby treating the sirtuin mediated disease or disorder.

78. A method for identifying a sirtuin modulating compound, comprising:
a) contacting a cell comprising a sirtuin sequence having at least one polymorphic variant with a test compound; and
b) determining (i) the level of expression from the sirtuin sequence, (ii) the level of activity of a sirtuin protein expressed by the sirtuin sequence, or (iii) both (i) and (ii), wherein a change in.(i), (ii) or both (i) and (ii) in the presence of the test compound as compared to a control is indicative of a compound that is a sirtuin modulating compound.

79-92. (canceled)

93. A method for identifying a sirtuin modulating compound, comprising:
a) contacting a cell comprising an expression construct with a test compound, wherein the expression construct comprises a reporter gene operably linked to a sirtuin promoter sequence having at least one polymorphic variant; and
b) determining the level of expression of the reporter gene, wherein a change in the level of expression of the reporter gene in the presence of the test compound as compared to a control is indicative of a compound that is a sirtuin modulating compound.

94-101. (canceled)
